(19) United States
(12) Patent Application Publication
(10) Pub. No.: US 2025/0115603 A1
(43) Pub. Date: Apr. 10, 2025

(54) CRYSTALLINE FORMS OF A KRAS INHIBITOR

(71) Applicant: Incyte Corporation, Wilmington, DE (US)

(72) Inventors: Jiemin Hu, Wilmington, DE (US); Zhongjiang Jia, Wilmington, DE (US); Trupti Sheth, Wilmington, DE (US); Naijing Su, Wilmington, DE (US); Huaping Zhang, Newark, DE (US)

(21) Appl. No.: 18/909,680

(22) Filed: Oct. 8, 2024

Related U.S. Application Data
(60) Provisional application No. 63/588,914, filed on Oct. 9, 2023.
(60) Provisional application No. 63/698,286, filed on Sep. 24, 2024.

(51) Int. Cl. C07D 471/04; A61K 9/20; A61K 31/4745; C07C 55/14; C07C 57/15; C07C 59/255

(52) U.S. Cl. CPC C07D 471/04; A61K 9/2013; A61K 9/2018; A61K 9/2054; A61K 9/2059; A61K 31/4745; C07C 55/14; C07C 57/15; C07C 59/255

(57) ABSTRACT

This disclosure provides salts, crystalline forms, and formulations of a KRAS inhibitor, and methods of making and using thereof.

### RELATED APPLICATIONS

This application is related to U.S. Provisional Application No. 63/588,914 filed Oct. 9, 2023, and U.S. Provisional Application No. 63/698,286 filed Sep. 24, 2024, the content of each is incorporated in its entirety.

### BACKGROUND

Ras proteins are part of the family of small GTPases that are activated by growth factors and various extracellular stimuli. The Ras family regulates intracellular signaling pathways responsible for growth, migration, survival and differentiation of cells. Activation of Ras proteins at the cell membrane results in the binding of key effectors and initiation of a cascade of intracellular signaling pathways within the cell, including the RAF and PI3K kinase pathways. Somatic mutations in RAS may result in uncontrolled cell growth and malignant transformation while the activation of RAS proteins is tightly regulated in normal cells (D. Simanshu, et al., Cell, 2017, 170 (1), 17-33).

The Ras family is comprised of three members: KRAS, NRAS and HRAS. RAS mutant cancers account for about 25% of human cancers. KRAS is the most frequently mutated isoform accounting for 85% of all RAS mutations whereas NRAS and HRAS are found mutated in 12% and 3% of all Ras mutant cancers respectively (D. Simanshu, et al., Cell, 2017, 170 (1), 17-33). KRAS mutations are prevalent amongst the top three most deadly cancer types: pancreatic (97%), colorectal (44%), and lung (30%) (A. D. Cox, et al. Nat. Rev. Drug. Discov., 2014, 13 (11), 828-51). The majority of RAS mutations occur at amino acid residue 12, 13, and 61. The frequency of specific mutations varies between RAS gene isoforms and while G12 and Q61 mutations are predominant in KRAS and NRAS respectively, G12, G13 and Q61 mutations are most frequent in HRAS. Furthermore, the spectrum of mutations in a RAS isoform differs between cancer types. For example, KRAS G12D mutations predominate in pancreatic cancers (51%), followed by colorectal adenocarcinomas (45%) and lung cancers (17%) while KRAS G12V mutations are associated with pancreatic cancers (30%), followed by colorectal adenocarcinomas (27%), and lung adenocarcinomas (23%) (A. D. Cox, et al. Nat. Rev. Drug. Discov., 2014, 13 (11), 828-51). In contrast, KRAS G12C mutations predominate in non-small cell lung cancer (NSCLC) comprising 11-16% of lung adenocarcinomas, and 2-5% of pancreatic and colorectal adenocarcinomas (A. D. Cox, et al. Nat. Rev. Drug. Discov., 2014, 13 (11), 828-51). Genomic studies across hundreds of cancer cell lines have demonstrated that cancer cells harboring KRAS mutations are highly dependent on KRAS function for cell growth and survival (R. McDonald, et al., Cell, 2017, 170 (3), 577-92). The role of mutant KRAS as an oncogenic driver is further supported by extensive in vivo experimental evidence showing mutant KRAS is required for early tumor onset and maintenance in animal models (A. D. Cox, et al. Nat. Rev. Drug. Discov., 2014, 13 (11), 828-51).

Taken together, these findings indicate that KRAS mutations play a critical role in human cancers. Development of inhibitors targeting KRAS, including mutant KRAS, will therefore be useful in the clinical treatment of diseases that are characterized by involvement of KRAS, including diseases characterized by the involvement or presence of a KRAS mutation.

### SUMMARY

Provided herein are crystalline forms, salts, and crystalline salt forms, of 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile having the following structure:

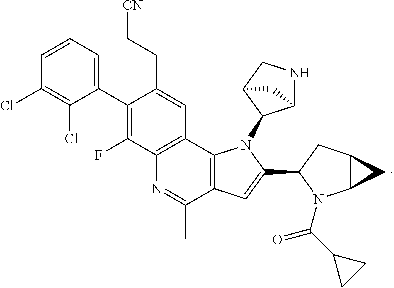 [SMILES: Cc1nc2c(F)c(-c3cccc(Cl)c3Cl)c(CCC#N)cc2c2c1cc([C@H]1C[C@H]3C[C@H]3N1C(=O)C1CC1)n2[C@H]1[C@H]2CN[C@@H]1C2]

This compound, referred to herein as “Compound 1,” is a KRAS inhibitor, particularly of KRAS having a G12D mutation, that can be used for the treatment of various cancers and other diseases.

### BRIEF DESCRIPTION OF THE DRAWINGS

FIG. 1 shows the XRPD diffractogram of Compound 1 hydrochloride dihydrate (Form I).

FIG. 2 shows the DSC thermogram of Compound 1 hydrochloride dihydrate (Form I).

FIG. 3 shows the TGA thermogram of Compound 1 hydrochloride dihydrate (Form I).

FIG. 4 shows the XRPD diffractogram of Compound 1 dihydrochloride.

FIG. 5 shows the DSC thermogram of Compound 1 dihydrochloride.

FIG. 6 shows the TGA thermogram of Compound 1 dihydrochloride.

FIG. 7 shows the XRPD diffractogram of Compound 1 fumarate.

FIG. 8 shows the DSC thermogram of Compound 1 fumarate.

FIG. 9 shows the TGA thermogram of Compound 1 fumarate.

FIG. 10 shows the XRPD diffractogram of Compound 1 L-tartrate.

FIG. 11 shows the DSC thermogram of Compound 1 L-tartrate.

FIG. 12 shows the TGA thermogram of Compound 1 L-tartrate.

FIG. 13 shows the XRPD diffractogram of Compound 1 adipate.

FIG. 14 shows the DSC thermogram of Compound 1 adipate.

FIG. 15 shows the TGA thermogram of Compound 1 adipate.

FIG. 16 shows the XRPD diffractogram of Compound 1 phosphate.

FIG. 17 shows the DSC thermogram of Compound 1 phosphate.

FIG. 18 shows the TGA thermogram of Compound 1 phosphate.

FIG. 19 shows the XRPD diffractogram of Compound 1 free base.

FIG. 20 shows the DSC thermogram of Compound 1 free base.

FIG. 21 shows the TGA thermogram of Compound 1 free base.

FIG. 22 shows the XRPD diffractogram of Compound 1 hydrochloride dihydrate (Form II).

FIG. 23 shows the XRPD diffractogram of Compound 1 hydrochloride dihydrate (Form III).

FIG. 24 shows the XRPD diffractogram of Compound 1 hydrochloride dihydrate (Form IV).

FIG. 25 shows the XRPD diffractogram of Compound 1 hydrochloride dihydrate (Form V).

FIG. 26 shows the XRPD diffractogram of Compound 1 hydrochloride dihydrate (Form VI).

FIG. 27 shows the XRPD diffractogram of Compound 1 hydrochloride dihydrate (Form VII).

FIG. 28 shows the XRPD diffractogram of Compound 1 hydrochloride dihydrate (Form VIII).

FIG. 29 shows the XRPD diffractogram of Compound 1 hydrochloride dihydrate (Form IX).

FIG. 30 shows an atomic displacement ellipsoid diagram of Compound 1 hydrochloride dihydrate.

### DETAILED DESCRIPTION

The solid state of a compound can be important when the compound is used for pharmaceutical purposes. The physical properties of a compound can change from one solid form to another, which can affect the suitability of the form for pharmaceutical use. For example, a particular crystalline solid compound can overcome the disadvantage of other solid forms of the compound such as, e.g., instability and/or reduced purity.

Provided herein are solid, crystalline forms and crystalline salt forms of 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, Compound 1. This compound is useful for the treatment of a variety of cancers.

Compound 1 is disclosed in PCT Application No. PCT/US2022/078048 (WO2023064857A1) and U.S. patent application Ser. No. 18/046,303 (US20230144051A1), the entire contents of which are incorporated herein by reference.

The crystalline forms provided herein can be characterized by X-ray powder diffraction (XRPD), differential scanning calorimetry (DSC), and thermogravimetric analysis (TGA).

### I. Definitions

Listed below are definitions of various terms used to describe the crystalline forms provided herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which the compound and its crystalline forms belong. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

The term “treat,” “treated,” “treating,” or “treatment” includes the diminishment or alleviation of at least one symptom associated or caused by the state, disorder or disease being treated. In certain embodiments, the treatment comprises bringing into contact with KRAS an effective amount of the compound provided herein for conditions related to androgen receptors.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. In an embodiment, the patient, subject, or individual is human.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The present disclosure also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in A. R. Gennaro (Ed.), Remington's Pharmaceutical Sciences, 17^{th} Ed., (Mack Publishing Company, Easton, 1985), p. 1418, S. M. Berge et al., J. Pharm. Sci., 1977, 66 (1), 1-19, S. Gaisford in A. Adejare (Ed.), Remington, The Science and Practice of Pharmacy, 23^{rd} Ed., (Elsevier, 2020), Chapter 17, pp. 307-14; S. M. Berge et al., J. Pharm. Sci., 1977, 66 (1), 1-19, T. S. Wiedmann, et al., Asian J. Pharm. Sci., 2016; 11, 722-34. D. Gupta et al., Molecules, 2018, 23 (7), 1719; P. H. Stahl et al., Handbook of Pharmaceutical Salts: Properties, Selection, and Use, (Wiley, 2002) and in P. H. Stahl et al., Handbook of Pharmaceutical Salts: Properties, Selection, and Use, 2^{nd} Ed. (Wiley, 2011).

The term “administering” or “administration” and the like, refers to providing a therapeutic agent, such as a crystalline form disclosed herein, to the subject in need of treatment. In an embodiment, the subject is a mammal. In another embodiment, the subject is a human.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±10%, including ±5%, ±1%, and ±0.1% from the specified value, as such variations are appropriate to perform the disclosed methods.

The following abbreviations may be used herein: AcOH (acetic acid); Ac₂O (acetic anhydride); aq. (aqueous); atm. (atmosphere(s)); Boc (t-butoxycarbonyl); BOP ((benzotriazol-1-yloxy)tris(dimethylamino)phosphonium hexafluorophosphate); br (broad); Cbz (carboxybenzyl); calc. (calculated); d (doublet); dd (doublet of doublets); DBU (1,8-diazabicyclo[5.4.0]undec-7-ene); DCM (dichloromethane); DIAD (N,N′-diisopropyl azidodicarboxylate); DIEA (N,N-diisopropylethylamine); DIPEA (N,N-diisopropylethylamine); DIBAL (diisobutylaluminium hydride); DMF (N,N-dimethylformamide); DMSO (dimethyl sulfoxide); DSC (differential scanning calorimetry); Et (ethyl); EtOAc (ethyl acetate); FCC (flash column chromatography); g (gram(s)); h (hour(s)); HATU (N,N,N′,N′-tetramethyl-O-(7-azabenzotriazol-1-yl) uronium hexafluorophosphate); HCl (hydrochloric acid); HPLC (high performance liquid chromatography); Hz (hertz); J (coupling constant); L (liter(s)); LCMS (liquid chromatography-mass spectrometry); LDA (lithium diisopropylamide); m (multiplet); M (molar); mCPBA (3-chloroperoxybenzoic acid); MS (Mass spectrometry); Me (methyl); MeCN (acetonitrile); MeOH (methanol); mg (milligram(s)); min. (minutes(s)); mL (milliliter(s)); mmol (millimole(s)); MTBE (methyl tert-butyl ether); N (normal); NCS (N-chlorosuccinimide); NEt₃ (triethylamine); nM (nanomolar); NMP (N-methylpyrrolidinone); NMR (nuclear magnetic resonance spectroscopy); OTf (trifluoromethanesulfonate); Ph (phenyl); pM (picomolar); PPT (precipitate); RP-HPLC (reverse phase high performance liquid chromatography); r.t. (room temperature), s (singlet); t (triplet or tertiary); TBS (tert-butyldimethylsilyl); tert (tertiary); tt (triplet of triplets); TFA (trifluoroacetic acid); THF (tetrahydrofuran); TGA (thermogravimetric analysis); μg (microgram(s)); μL (microliter(s)); μM (micromolar); wt % (weight percent); XRPD (X-ray powder diffraction). Brine is saturated aqueous sodium chloride. In vacuo is under vacuum.

Compounds of the present disclosure can exist in the form of atropisomers (i.e., conformational diastereoisomers) that can be stable at ambient temperature and separable, e.g., by chromatography. For example, compounds provided herein can exist in the form of atropisomers in which the conformation of the dichlorophenyl relative to the remainder of the molecule is as shown by the partial formulae Formula (II-A) or Formula (II-B) below. Reference to the compounds described herein or any of the embodiments is understood to include all such atropisomeric forms of the compounds, including, without limitation, the atropisomeric forms represented by Formula (II-A) or Formula (II-B) below. The asymmetry of atropisomers is assigned as either Rₐ or Sₐ, as determined by conventional methods of characterizing points of asymmetry.

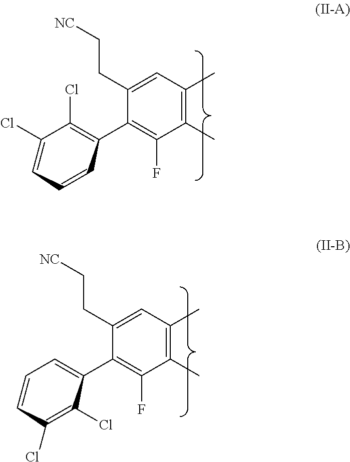

### II. Salts and Crystalline Forms

### 1. Characterization of Crystalline Forms

In certain embodiments, the crystalline forms described herein are identifiable on the basis of characteristic peaks in an X-ray powder diffraction analysis. X-ray powder diffraction (XRPD) is a scientific technique using X-ray, neutron, or electron diffraction on powder, microcrystalline, or other solid materials for structural characterization of solid materials. A description of the methods used to obtain certain XRPD diffractograms in connection with the crystalline forms provided herein can be found in the Examples below. In an embodiment, the X-ray powder diffraction data provided herein is obtained by a method utilizing Cu Kα radiation.

Thus, in an aspect, provided herein is a compound that is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, wherein the compound is crystalline.

In an embodiment, the compound or pharmaceutically acceptable salt thereof is a pharmaceutically acceptable salt thereof. In another embodiment, the pharmaceutically acceptable salt is selected from the group consisting of hydrochloride, di-hydrochloride, fumarate, L-tartrate, adipate, and phosphate.

In another embodiment, the compound or pharmaceutically acceptable salt thereof is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile:

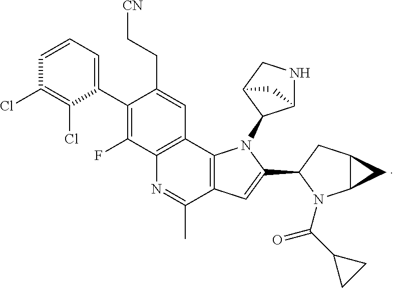

In yet another embodiment, the compound or pharmaceutically acceptable salt thereof is 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile:

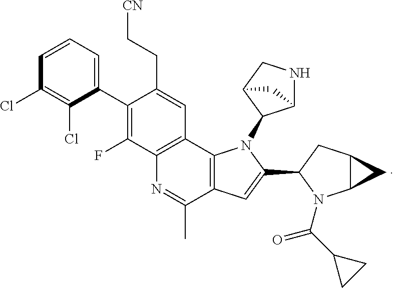

In an embodiment, the compound or pharmaceutically acceptable salt thereof is a solvate. In another embodiment, the compound or pharmaceutically acceptable salt thereof is a hydrate. In another embodiment, the compound or pharmaceutically acceptable salt thereof is a dihydrate.

### Compound 1 Hydrochloride Dihydrate

In an embodiment, the compound or pharmaceutically acceptable salt thereof is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate.

In an embodiment, the compound or pharmaceutically acceptable salt thereof is Form I.

In another embodiment, the compound or pharmaceutically acceptable salt thereof is characterized by an XRPD diffractogram having at least one (or two) of the following peaks expressed in degrees-2-theta at angles (±0.2 degrees) of 5.7, 7.8, 13.5, 17.2, 19.2, 20.5, 22.8, 23.1, 23.5, and 26.0.

In yet another embodiment, the compound or pharmaceutically acceptable salt thereof is characterized by an XRPD diffractogram having at least three (or at least four, five, six, seven, eight, nine, or all) of the following peaks expressed in degrees-2-theta at angles (±0.2 degrees) of 5.7, 7.8, 13.5, 17.2, 19.2, 20.5, 22.8, 23.1, 23.5, and 26.0.

In still another embodiment, the compound or pharmaceutically acceptable salt thereof is characterized by an XRPD diffractogram having peaks expressed in degrees-2-theta at angles (±0.2 degrees) of 5.7, 7.8, and 13.5.

In still another embodiment, the compound or pharmaceutically acceptable salt thereof is characterized by an XRPD diffractogram having peaks expressed in degrees-2-theta at angles (±0.2 degrees) of 5.7, 7.8, 13.5, and 17.2.

In an embodiment, the compound or pharmaceutically acceptable salt thereof is characterized by an XRPD diffractogram having at least six of the following peaks expressed in degrees-2-theta at angles (±0.2 degrees) of 5.7, 7.8, 13.5, 17.2, 19.2, 20.5, 22.8, 23.1, 23.5, and 26.0. In another embodiment, the compound or pharmaceutically acceptable salt thereof is characterized by an XRPD diffractogram having peaks expressed in degrees-2-theta at angles (±0.2 degrees) of 5.7, 7.8, 13.5, 17.2, 19.2, and 23.1.

In yet another embodiment, the compound or pharmaceutically acceptable salt thereof is characterized by an XRPD diffractogram having peaks expressed in degrees-2-theta at angles (±0.2 degrees) of 5.7, 7.8, 13.5, 17.2, 19.2, 20.5, 22.8, 23.1, 23.5, and 26.0.

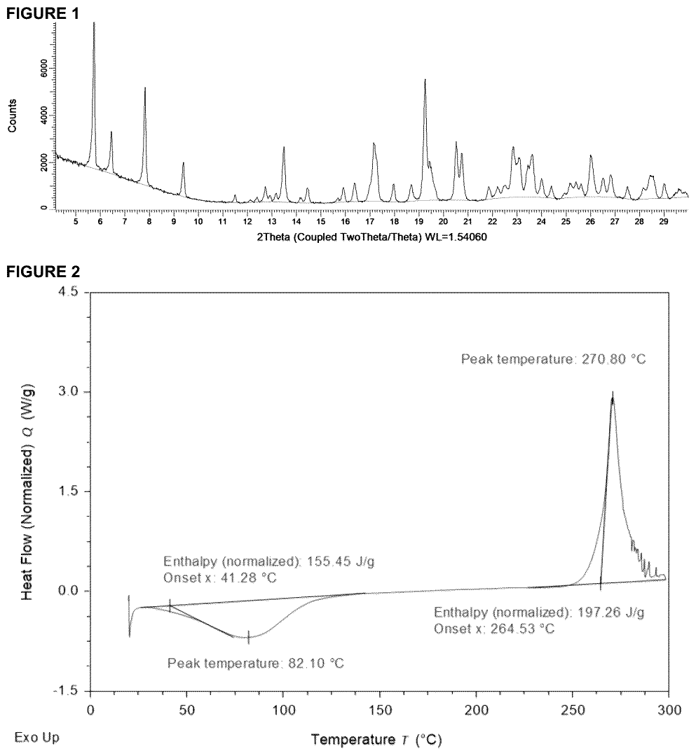

In another embodiment, the compound or pharmaceutically acceptable salt thereof is characterized by an XRPD diffractogram substantially as depicted in FIG. 1.

In another embodiment, the compound or pharmaceutically acceptable salt thereof is characterized by an XRPD diffractogram having peaks expressed in degrees-2-theta at angles (±0.2 degrees) selected from the list in Table 1a (Form I).

TABLE 1a

| 2-Theta (°) | H % |
|---|---|
| 5.7 | 100 |
| 6.4 | 24.7 |
| 7.8 | 56.5 |
| 9.4 | 20.0 |
| 11.5 | 4.2 |
| 12.1 | 1.7 |
| 12.4 | 3.0 |
| 12.7 | 8.8 |
| 12.9 | 3.7 |
| 13.2 | 4.9 |
| 13.5 | 32.0 |
| 14.2 | 3.2 |
| 14.5 | 8.8 |
| 15.7 | 2.6 |
| 15.9 | 8.4 |
| 16.4 | 10.4 |
| 17.2 | 34.0 |
| 18.0 | 10.0 |
| 18.7 | 9.8 |
| 19.2 | 70.5 |
| 19.4 | 22.6 |
| 20.5 | 33.7 |
| 20.7 | 27.2 |
| 21.9 | 7.3 |
| 22.2 | 6.8 |
| 22.5 | 7.2 |
| 22.8 | 29.4 |
| 23.1 | 22.7 |
| 23.5 | 17.5 |
| 23.6 | 24.8 |
| 24.0 | 11.1 |
| 24.4 | 7.0 |
| 25.0 | 2.1 |
| 25.2 | 7.6 |
| 25.4 | 9.1 |
| 25.6 | 7.7 |
| 26.0 | 23.8 |
| 26.5 | 10.8 |
| 26.8 | 13.2 |
| 27.5 | 7.3 |
| 28.2 | 6.3 |
| 28.5 | 13.0 |
| 29.0 | 8.3 |
| 29.6 | 4.7 |

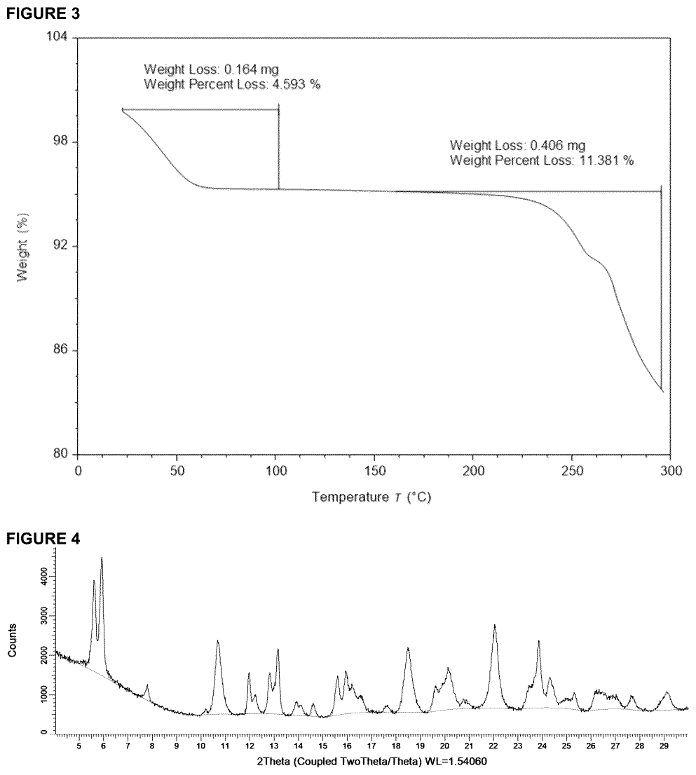

In another embodiment, the compound or pharmaceutically acceptable salt thereof has a DSC thermogram characterized by an endotherm substantially as depicted in FIG. 2. In another embodiment, the compound or pharmaceutically acceptable salt thereof has a DSC thermogram characterized by an exotherm having a peak at about 271° C. In another embodiment, the compound or pharmaceutically acceptable salt thereof has a DSC thermogram characterized by an exotherm having an onset at about 265° C. In another embodiment, the compound or pharmaceutically acceptable salt thereof has a DSC thermogram characterized by a dehydration event below about 150° C. In yet another embodiment, the compound or pharmaceutically acceptable salt thereof has a TGA thermogram substantially as depicted in FIG. 3.

In an embodiment, the compound or pharmaceutically acceptable salt thereof comprises crystals that are monoclinic. In another embodiment, the compound or pharmaceutically acceptable salt thereof comprises crystals wherein the space group is P21. In yet another embodiment, the compound or pharmaceutically acceptable salt thereof comprises crystals having cell parameters of substantially a=13.8490 (4) Å, b=8.0204 (2) Å, c=15.5461 (4) Å, α=90°, β=100.617 (3)°, γ=90°, and/or V=1697.21 (8) Å³. In still another embodiment, the compound or pharmaceutically acceptable salt thereof comprises crystals having cell parameters substantially as shown in Table A.

In an embodiment, the compound or pharmaceutically acceptable salt thereof is Form II. In another embodiment, the compound or pharmaceutically acceptable salt thereof is characterized by an XRPD diffractogram having at least one (or two) of the following peaks expressed in degrees-2-theta at angles (±0.2 degrees) of 5.8, 7.6, 11.4, 12.5, 14.4, 17.9, and 25.3.

In yet another embodiment, the compound or pharmaceutically acceptable salt thereof is characterized by an XRPD diffractogram having at least three (or at least four, five, six, or all) of the following peaks expressed in degrees-2-theta at angles (±0.2 degrees) of 5.8, 7.6, 11.4, 12.5, 14.4, 17.9, and 25.3.

In still another embodiment, the compound or pharmaceutically acceptable salt thereof is characterized by an XRPD diffractogram having peaks expressed in degrees-2-theta at angles (±0.2 degrees) of 7.6, 12.5, and 17.9.

In still another embodiment, the compound or pharmaceutically acceptable salt thereof is characterized by an XRPD diffractogram having peaks expressed in degrees-2-theta at angles (±0.2 degrees) of 7.6, 12.5, 17.9, and 25.3.

In an embodiment, the compound or pharmaceutically acceptable salt thereof is characterized by an XRPD diffractogram having at least six of the following peaks expressed in degrees-2-theta at angles (±0.2 degrees) of 55.8, 7.6, 11.4, 12.5, 14.4, 17.9, and 25.3. In another embodiment, the compound or pharmaceutically acceptable salt thereof is characterized by an XRPD diffractogram having peaks expressed in degrees-2-theta at angles (±0.2 degrees) of 5.8, 7.6, 11.4, 12.5, 14.4, 17.9, and 25.3.

In another embodiment, the compound or pharmaceutically acceptable salt thereof is characterized by an XRPD diffractogram substantially as depicted in FIG. 22.

In another embodiment, the compound or pharmaceutically acceptable salt thereof is characterized by an XRPD diffractogram having peaks expressed in degrees-2-theta at angles (±0.2 degrees) selected from the list in Table 1b (Form II).

TABLE 1b

| 2-Theta (°) | H % |
|---|---|
| 5.8 | 17.9 |
| 7.6 | 26.1 |
| 11.4 | 18.2 |
| 12.5 | 100 |
| 14.4 | 25.4 |
| 16.2 | 6.0 |
| 17.2 | 13.0 |
| 17.9 | 29.2 |
| 20.4 | 7.7 |
| 21.4 | 8.4 |
| 23.3 | 1.3 |
| 25.3 | 21.0 |
| 27.1 | 6.6 |
| 28.0 | 0.4 |

In an embodiment, the compound or pharmaceutically acceptable salt thereof is Form III. In another embodiment, the compound or pharmaceutically acceptable salt thereof is characterized by an XRPD diffractogram substantially as depicted in FIG. 23.

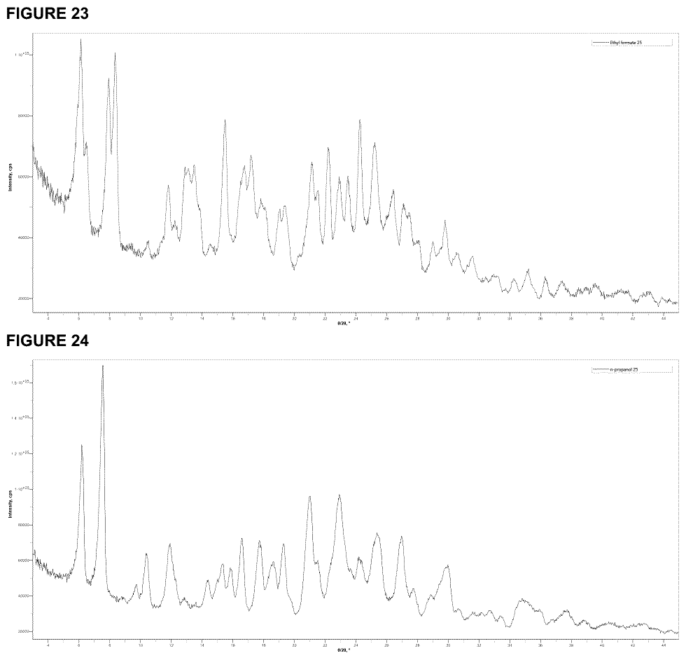

In an embodiment, the compound or pharmaceutically acceptable salt thereof is Form IV. In another embodiment, the compound or pharmaceutically acceptable salt thereof is characterized by an XRPD diffractogram substantially as depicted in FIG. 24.

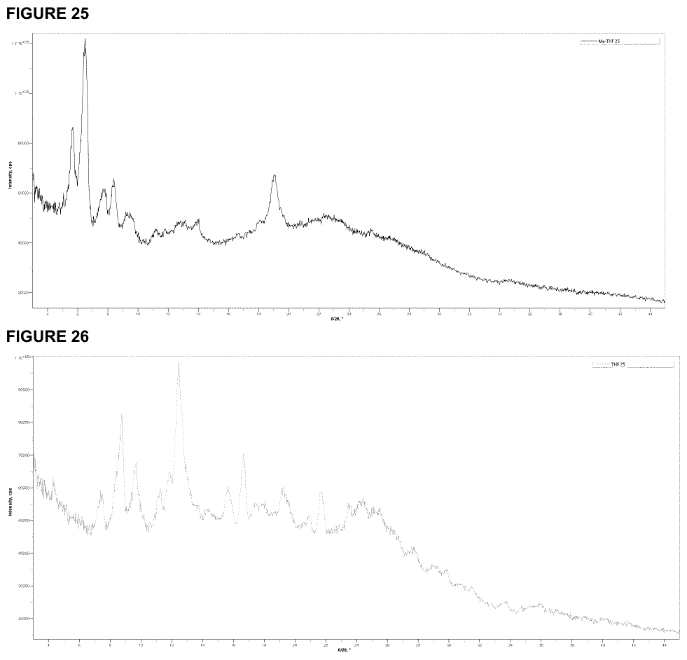

In an embodiment, the compound or pharmaceutically acceptable salt thereof is Form V. In another embodiment, the compound or pharmaceutically acceptable salt thereof is characterized by an XRPD diffractogram substantially as depicted in FIG. 25.

In an embodiment, the compound or pharmaceutically acceptable salt thereof is Form VI. In another embodiment, the compound or pharmaceutically acceptable salt thereof is characterized by an XRPD diffractogram substantially as depicted in FIG. 26.

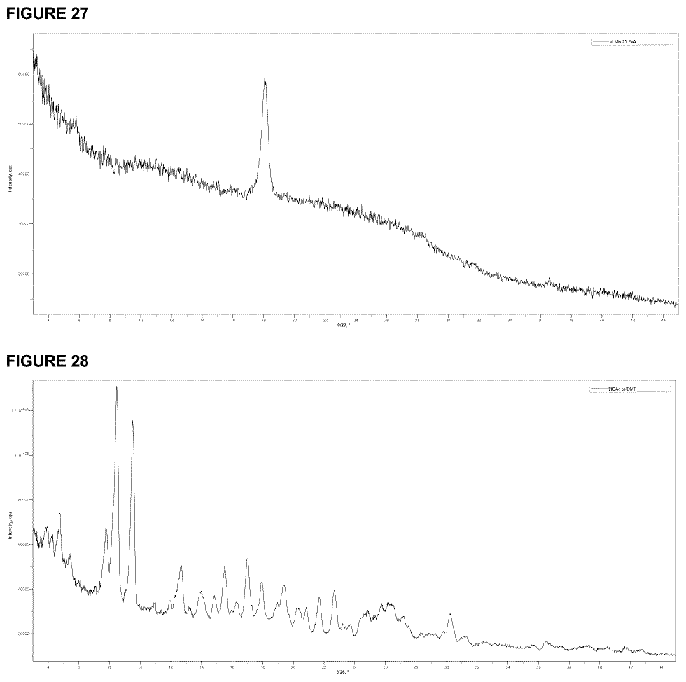

In an embodiment, the compound or pharmaceutically acceptable salt thereof is Form VII. In another embodiment, the compound or pharmaceutically acceptable salt thereof is characterized by an XRPD diffractogram substantially as depicted in FIG. 27.

In an embodiment, the compound or pharmaceutically acceptable salt thereof is Form VIII. In another embodiment, the compound or pharmaceutically acceptable salt thereof is characterized by an XRPD diffractogram substantially as depicted in FIG. 28.

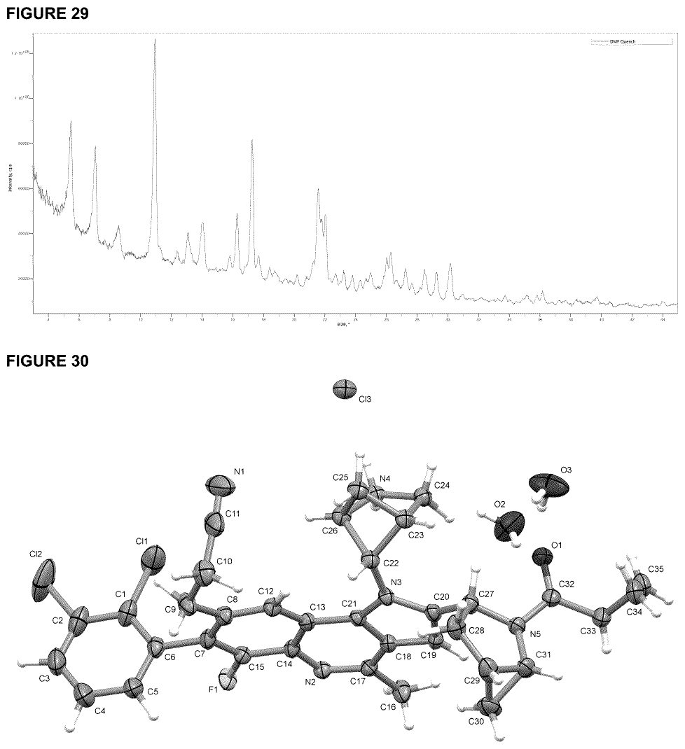

In an embodiment, the compound or pharmaceutically acceptable salt thereof is Form IX. In another embodiment, the compound or pharmaceutically acceptable salt thereof is characterized by an XRPD diffractogram substantially as depicted in FIG. 29.

### Compound 1 Dihydrochloride

In an embodiment, the compound or pharmaceutically acceptable salt thereof is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile dihydrochloride.

In another embodiment, the compound or pharmaceutically acceptable salt thereof is characterized by an XRPD diffractogram having at least one (or two) of the following peaks expressed in degrees-2-theta at angles (±0.2 degrees) of 5.6, 5.9, 10.7, 12.0, 12.8, 13.2, 15.6, 15.9, 18.5, 20.1, 22.1, and 23.9.

In yet another embodiment, the compound or pharmaceutically acceptable salt thereof is characterized by an XRPD diffractogram having at least three (or four, five, six, seven, eight, nine, ten, eleven, or all) of the following peaks expressed in degrees-2-theta at angles (±0.2 degrees) of 5.6, 5.9, 10.7, 12.0, 12.8, 13.2, 15.6, 15.9, 18.5, 20.1, 22.1, and 23.9.

In still another embodiment, the compound or pharmaceutically acceptable salt thereof is characterized by an XRPD diffractogram having peaks expressed in degrees-2-theta at angles (±0.2 degrees) of 5.6, 5.9, and 22.1. In an embodiment, the compound or pharmaceutically acceptable salt thereof is characterized by an XRPD diffractogram having at least seven peaks expressed in degrees-2-theta at angles (±0.2 degrees) of 5.6, 5.9, 10.7, 12.0, 12.8, 13.2, 15.6, 15.9, 18.5, 20.1, 22.1, and 23.9.

In another embodiment, the compound or pharmaceutically acceptable salt thereof is characterized by an XRPD diffractogram having peaks expressed in degrees-2-theta at angles (±0.2 degrees) of 5.6, 5.9, 10.7, 13.2, 18.5, 22.1, and 23.9. In yet another embodiment, the compound or pharmaceutically acceptable salt thereof is characterized by an XRPD diffractogram having peaks expressed in degrees-2-theta at angles (±0.2 degrees) of 5.6, 5.9, 10.7, 12.0, 12.8, 13.2, 15.6, 15.9, 18.5, 20.1, 22.1, and 23.9.

In another embodiment, the compound or pharmaceutically acceptable salt thereof is characterized by an XRPD diffractogram substantially as depicted in FIG. 4.

In another embodiment, the compound or pharmaceutically acceptable salt thereof is characterized by an XRPD diffractogram having peaks expressed in degrees-2-theta at angles (±0.2 degrees) selected from the list in Table 2.

TABLE 2

| 2-Theta (°) | H% |
|---|---|
| 5.6 | 77.1 |
| 5.9 | 100 |
| 7.8 | 13.0 |
| 10.2 | 4.4 |
| 10.7 | 62.0 |
| 12.0 | 35.0 |
| 12.2 | 15.8 |
| 12.8 | 34.5 |
| 13.2 | 55.0 |
| 13.9 | 11.6 |
| 14.1 | 8.5 |
| 14.6 | 11.1 |
| 15.6 | 33.2 |
| 15.9 | 36.0 |
| 16.2 | 23.9 |
| 16.5 | 14.5 |
| 17.6 | 5.7 |
| 18.5 | 54.9 |
| 19.6 | 19.9 |
| 20.1 | 33.7 |
| 20.8 | 5.3 |
| 20.9 | 7.7 |
| 22.1 | 68.2 |
| 23.5 | 19.3 |
| 23.9 | 57.3 |
| 24.3 | 25.6 |
| 25.0 | 8.7 |
| 25.3 | 14.3 |
| 26.3 | 14.5 |
| 26.9 | 11.5 |
| 27.6 | 12.0 |
| 28.8 | 6.2 |
| 29.1 | 14.5 |

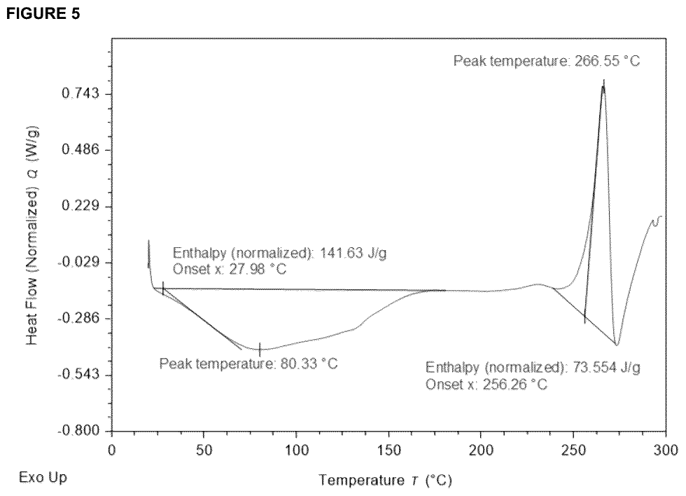

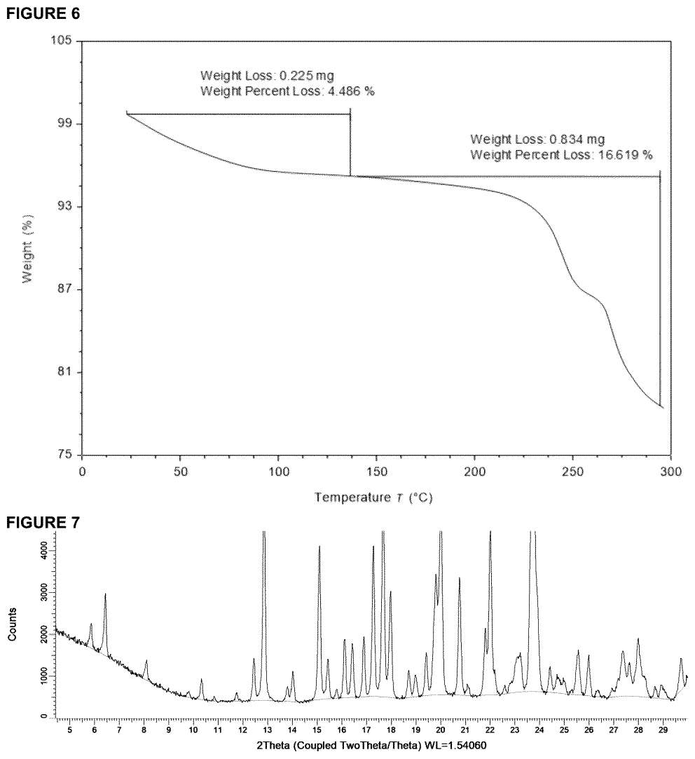

In another embodiment, the compound or pharmaceutically acceptable salt thereof has a DSC thermogram characterized by an endotherm substantially as depicted in FIG. 5. In yet another embodiment, the compound or pharmaceutically acceptable salt thereof has a DSC thermogram characterized by an exotherm having a peak at about 267° C. In another embodiment, the compound or pharmaceutically acceptable salt thereof has a DSC thermogram characterized by an exotherm having an onset at about 256° C. In another embodiment, the compound or pharmaceutically acceptable salt thereof has a DSC thermogram characterized by a dehydration event below about 150° C. In yet another embodiment, the compound or pharmaceutically acceptable salt thereof has a TGA thermogram substantially as depicted in FIG. 6.

### Compound 1 Fumarate

In an embodiment, the compound or pharmaceutically acceptable salt thereof is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile fumarate.

In another embodiment, the compound or pharmaceutically acceptable salt thereof is characterized by an XRPD diffractogram having at least one (or two) of the following peaks expressed in degrees-2-theta at angles (±0.2 degrees) of 12.9, 15.1, 17.3, 17.7, 19.8, 20.0, 20.8, 22.0, and 23.7.

In yet another embodiment, the compound or pharmaceutically acceptable salt thereof is characterized by an XRPD diffractogram having at least three (or four, five, six, seven, eight, or all) of the following peaks expressed in degrees-2-theta at angles (±0.2 degrees) of 12.9, 15.1, 17.3, 17.7, 19.8, 20.0, 20.8, 22.0, and 23.7.

In still another embodiment, the compound or pharmaceutically acceptable salt thereof is characterized by an XRPD diffractogram having peaks expressed in degrees-2-theta at angles (±0.2 degrees) of 12.9, 17.7, and 23.7. In an embodiment, the compound or pharmaceutically acceptable salt thereof is characterized by an XRPD diffractogram having at least six peaks expressed in degrees-2-theta at angles (±0.2 degrees) of 12.9, 15.1, 17.3, 17.7, 19.8, 20.0, 20.8, 22.0, and 23.7.

In another embodiment, the compound or pharmaceutically acceptable salt thereof is characterized by an XRPD diffractogram having peaks expressed in degrees-2-theta at angles (±0.2 degrees) of 12.9, 15.1, 17.7, 20.0, 22.0, and 23.7. In yet another embodiment, the compound or pharmaceutically acceptable salt thereof is characterized by an XRPD diffractogram having at least nine peaks expressed in degrees-2-theta at angles (±0.2 degrees) of 12.9, 15.1, 17.3, 17.7, 19.8, 20.0, 20.8, 22.0, and 23.7.

In still another embodiment, the compound or pharmaceutically acceptable salt thereof of claim 46, which is characterized by an XRPD diffractogram having peaks expressed in degrees-2-theta at angles (±0.2 degrees) of 12.9, 15.1, 17.3, 17.7, 19.8, 20.0, 20.8, 22.0, and 23.7.

In another embodiment, the compound or pharmaceutically acceptable salt thereof is characterized by an XRPD diffractogram substantially as depicted in FIG. 7.

In another embodiment, the compound or pharmaceutically acceptable salt thereof is characterized by an XRPD diffractogram having peaks expressed in degrees-2-theta at angles (±0.2 degrees) selected from the list in Table 3.

TABLE 3

| 2-Theta (°) | H % |
|---|---|
| 5.9 | 8.0 |
| 6.4 | 20.9 |
| 8.1 | 6.5 |
| 9.8 | 2.1 |
| 10.3 | 7.0 |
| 10.8 | 1.7 |
| 11.7 | 2.5 |
| 12.4 | 14.2 |
| 12.9 | 76.4 |
| 13.8 | 5.0 |
| 14.0 | 10.3 |
| 15.1 | 51.8 |
| 15.4 | 13.3 |
| 15.8 | 3.0 |
| 16.1 | 19.7 |
| 16.4 | 17.8 |
| 16.9 | 19.9 |
| 17.3 | 50.8 |
| 17.7 | 72.6 |
| 18.0 | 35.8 |
| 18.7 | 9.1 |
| 19.0 | 7.3 |
| 19.4 | 14.0 |
| 19.8 | 40.7 |
| 20.0 | 62.6 |
| 20.8 | 39.5 |
| 21.1 | 3.4 |
| 21.8 | 22.5 |
| 22.0 | 56.0 |
| 22.6 | 3.0 |
| 22.9 | 3.3 |
| 23.2 | 11.5 |
| 23.7 | 100 |
| 24.4 | 8.2 |
| 24.7 | 4.9 |
| 25.0 | 4.6 |
| 25.6 | 14.7 |
| 26.0 | 13.4 |
| 26.4 | 2.1 |
| 26.9 | 2.7 |
| 27.4 | 15.3 |
| 27.6 | 10.8 |
| 28.0 | 19.5 |
| 28.2 | 6.8 |
| 28.7 | 3.9 |
| 29.0 | 4.5 |
| 29.7 | 10.4 |

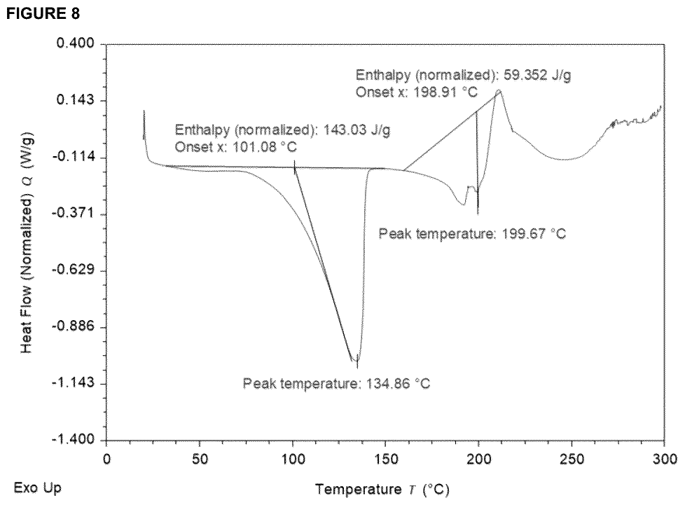

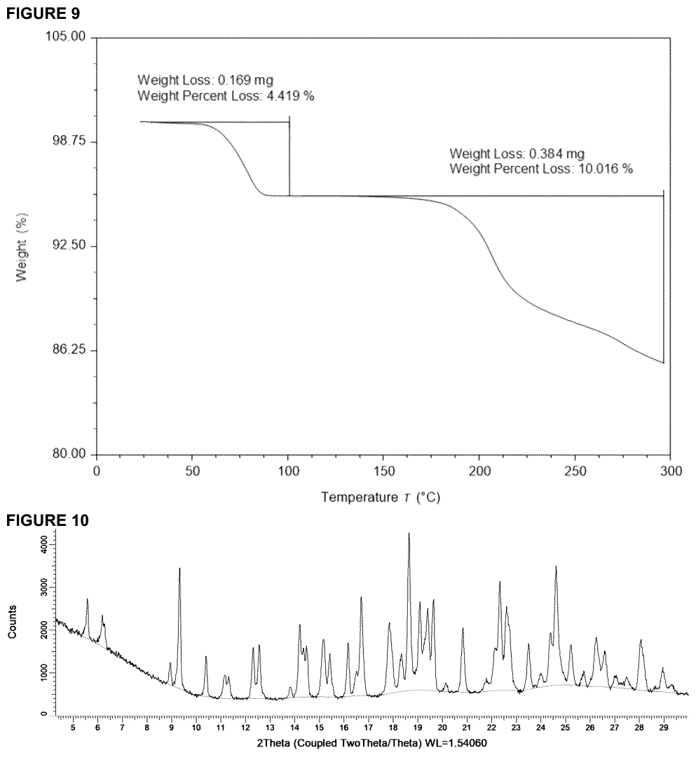

In another embodiment, the compound or pharmaceutically acceptable salt thereof has a DSC thermogram characterized by an endotherm substantially as depicted in FIG. 8. In yet another embodiment, compound or pharmaceutically acceptable salt thereof has a DSC thermogram characterized by an exotherm having a peak at about 200° C. In another embodiment, compound or pharmaceutically acceptable salt thereof has a DSC thermogram characterized by an exotherm having an onset at about 199° C. In another embodiment, the compound or pharmaceutically acceptable salt thereof has a DSC thermogram characterized by a dehydration event below about 150° C. In still another embodiment, the compound or pharmaceutically acceptable salt thereof has a TGA thermogram substantially as depicted in FIG. 9.

### Compound 1 L-Tartrate

In an embodiment, the compound or pharmaceutically acceptable salt thereof is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile L-tartrate.

In another embodiment, the compound or pharmaceutically acceptable salt thereof is characterized by an XRPD diffractogram having at least one (or two) of the following peaks expressed in degrees-2-theta at angles (±0.2 degrees) of 9.3, 16.7, 18.6, 19.1, 19.4, 19.6, 22.3, 22.6, and 24.6.

In yet another embodiment, the compound or pharmaceutically acceptable salt thereof is characterized by an XRPD diffractogram having at least three (or four, five, six, seven, eight, or all) of the following peaks expressed in degrees-2-theta at angles (±0.2 degrees) of 9.3, 16.7, 18.6, 19.1, 19.4, 19.6, 22.3, 22.6, and 24.6.

In still another embodiment, the compound or pharmaceutically acceptable salt thereof is characterized by an XRPD diffractogram having peaks expressed in degrees-2-theta at angles (±0.2 degrees) of 9.3, 18.6, and 24.6. In an embodiment, the compound or pharmaceutically acceptable salt thereof is characterized by an XRPD diffractogram having at least six peaks expressed in degrees-2-theta at angles (±0.2 degrees) of 9.3, 16.7, 18.6, 19.1, 19.4, 19.6, 22.3, 22.6, and 24.6.

In another embodiment, the compound or pharmaceutically acceptable salt thereof is characterized by an XRPD diffractogram having peaks expressed in degrees-2-theta at angles (±0.2 degrees) of 9.3, 16.7, 18.6, 19.6, 22.3, and 24.6. In yet another embodiment, the compound or pharmaceutically acceptable salt thereof is characterized by an XRPD diffractogram having at least nine peaks expressed in degrees-2-theta at angles (±0.2 degrees) of 9.3, 16.7, 18.6, 19.1, 19.4, 19.6, 22.3, 22.6, and 24.6.

In another embodiment, the compound or pharmaceutically acceptable salt thereof is characterized by an XRPD diffractogram substantially as depicted in FIG. 10.

In another embodiment, the compound or pharmaceutically acceptable salt thereof is characterized by an XRPD diffractogram having peaks expressed in degrees-2-theta at angles (±0.2 degrees) selected from the list in Table 4.

TABLE 4

| 2-Theta | H % |
|---|---|
| 5.6 | 24.9 |
| 6.2 | 19.7 |
| 8.9 | 14.3 |
| 9.3 | 76.4 |
| 10.4 | 25.3 |
| 11.2 | 14.7 |
| 11.3 | 13.6 |
| 12.3 | 32.2 |
| 12.6 | 33.7 |
| 13.8 | 6.5 |
| 14.2 | 45.9 |
| 14.5 | 31.6 |
| 15.2 | 36.1 |
| 15.4 | 27.4 |
| 16.2 | 33.9 |
| 16.5 | 15.7 |
| 16.7 | 62.7 |
| 17.8 | 45.0 |
| 18.3 | 23.3 |
| 18.6 | 100 |
| 19.1 | 55.6 |
| 19.4 | 51.5 |
| 19.6 | 57.5 |
| 20.1 | 5.5 |
| 20.8 | 40.5 |
| 21.8 | 7.9 |
| 22.1 | 26.9 |
| 22.3 | 69.1 |
| 22.6 | 53.1 |
| 23.5 | 28.9 |
| 24.0 | 9.0 |
| 24.4 | 33.5 |
| 24.6 | 75.5 |
| 25.2 | 25.5 |
| 25.7 | 7.1 |
| 26.3 | 31.0 |
| 26.6 | 22.0 |
| 27.0 | 6.4 |
| 27.5 | 6.9 |
| 28.1 | 31.9 |
| 28.9 | 14.8 |
| 29.3 | 5.2 |

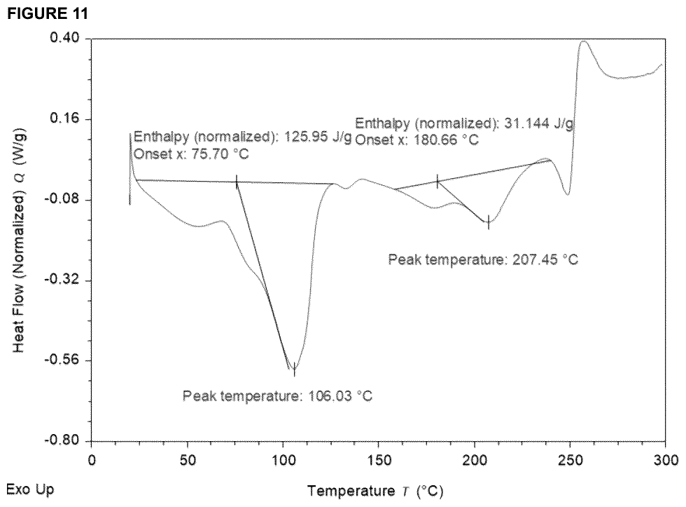

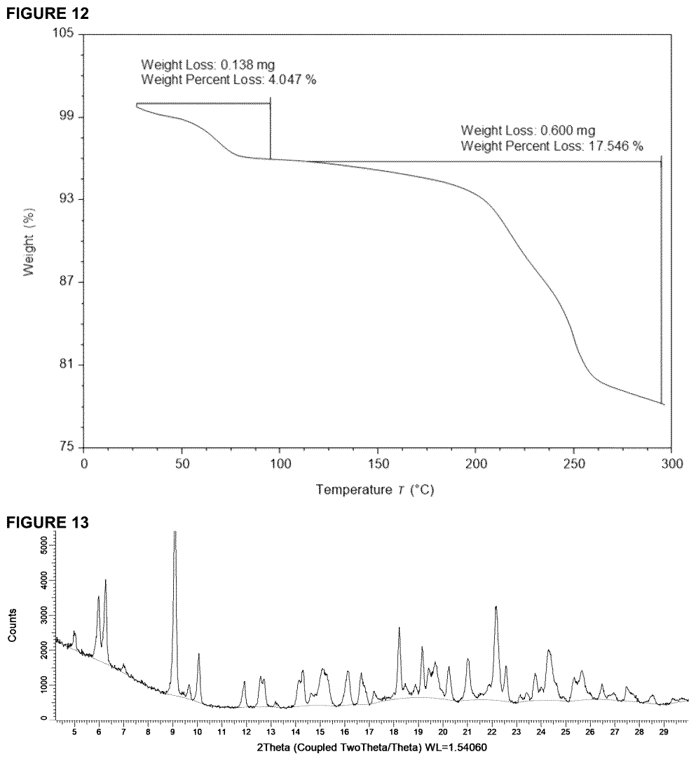

In another embodiment, the compound or pharmaceutically acceptable salt thereof has a DSC thermogram characterized by an endotherm substantially as depicted in FIG. 11. In yet another embodiment, the compound or pharmaceutically acceptable salt thereof has a DSC thermogram characterized by an endotherm having a peak at about 208° C. In another embodiment, the compound or pharmaceutically acceptable salt thereof has a DSC thermogram characterized by an endotherm having an onset at about 181° C. In another embodiment, the compound or pharmaceutically acceptable salt thereof has a DSC thermogram characterized by a dehydration event below about 150° C. In still another embodiment, the compound or pharmaceutically acceptable salt thereof has a TGA thermogram substantially as depicted in FIG. 12.

### Compound 1 Adipate

In an embodiment, the compound or pharmaceutically acceptable salt thereof is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile adipate.

In another embodiment, the compound or pharmaceutically acceptable salt thereof is characterized by an XRPD diffractogram having at least one (or two) of the following peaks expressed in degrees-2-theta at angles (±0.2 degrees) of 6.3, 9.1, 10.1, 18.2, 19.2, 24.3, 34.1, and 49.8.

In yet another embodiment, the compound or pharmaceutically acceptable salt thereof is characterized by an XRPD diffractogram having at least three (or four, five, six, seven, or all) of the following peaks expressed in degrees-2-theta at angles (±0.2 degrees) of 6.3, 9.1, 10.1, 18.2, 19.2, 24.3, 34.1, and 49.8.

In still another embodiment, the compound or pharmaceutically acceptable salt thereof is characterized by an XRPD diffractogram having peaks expressed in degrees-2-theta at angles (±0.2 degrees) of 6.3, 9.1, and 24.3. In an embodiment, the compound or pharmaceutically acceptable salt thereof is characterized by an XRPD diffractogram having at least six peaks expressed in degrees-2-theta at angles (±0.2 degrees) of 6.3, 9.1, 10.1, 18.2, 19.2, 24.3, 34.1, and 49.8.

In another embodiment, the compound or pharmaceutically acceptable salt thereof is characterized by an XRPD diffractogram having peaks expressed in degrees-2-theta at angles (±0.2 degrees) of 6.3, 9.1, 18.2, 19.2, 34.1, and 49.8. In yet another embodiment, the compound or pharmaceutically acceptable salt thereof is characterized by an XRPD diffractogram having peaks expressed in degrees-2-theta at angles (±0.2 degrees) of 6.3, 9.1, 18.2, 19.2, 34.1, and 49.8.

In another embodiment, the compound or pharmaceutically acceptable salt thereof is characterized by an XRPD diffractogram substantially as depicted in FIG. 13.

In another embodiment, the compound or pharmaceutically acceptable salt thereof is characterized by an XRPD diffractogram having peaks expressed in degrees-2-theta at angles (±0.2 degrees) selected from the list in Table 5.

TABLE 5

| 2-Theta (°) | H % |
|---|---|
| 5.0 | 8.8 |
| 6.0 | 34.1 |
| 6.3 | 44.2 |
| 7.0 | 5.0 |
| 9.1 | 100 |
| 9.7 | 7.5 |
| 10.1 | 25.4 |
| 11.9 | 13.6 |
| 12.6 | 12.5 |
| 13.2 | 2.2 |
| 14.1 | 13.6 |
| 14.3 | 18.4 |
| 14.7 | 5.5 |
| 15.1 | 19.4 |
| 16.1 | 18.1 |
| 16.7 | 16.5 |
| 17.2 | 4.9 |
| 18.2 | 37.1 |
| 18.5 | 7.9 |
| 18.9 | 7.0 |
| 19.2 | 26.3 |
| 19.5 | 13.8 |
| 19.7 | 19.4 |
| 20.2 | 17.6 |
| 21.0 | 21.7 |
| 21.9 | 7.2 |
| 22.2 | 49.8 |
| 22.6 | 18.7 |
| 23.2 | 3.9 |
| 23.4 | 4.4 |
| 23.8 | 14.3 |
| 24.3 | 26.0 |
| 24.9 | 1.8 |
| 25.4 | 12.3 |
| 25.7 | 15.6 |
| 26.5 | 8.3 |
| 26.9 | 3.4 |
| 27.5 | 6.2 |
| 27.8 | 4.0 |
| 28.5 | 4.6 |
| 29.4 | 1.7 |

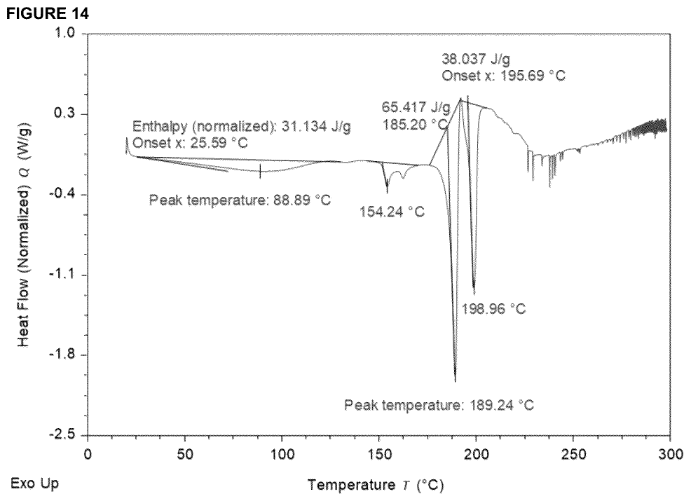

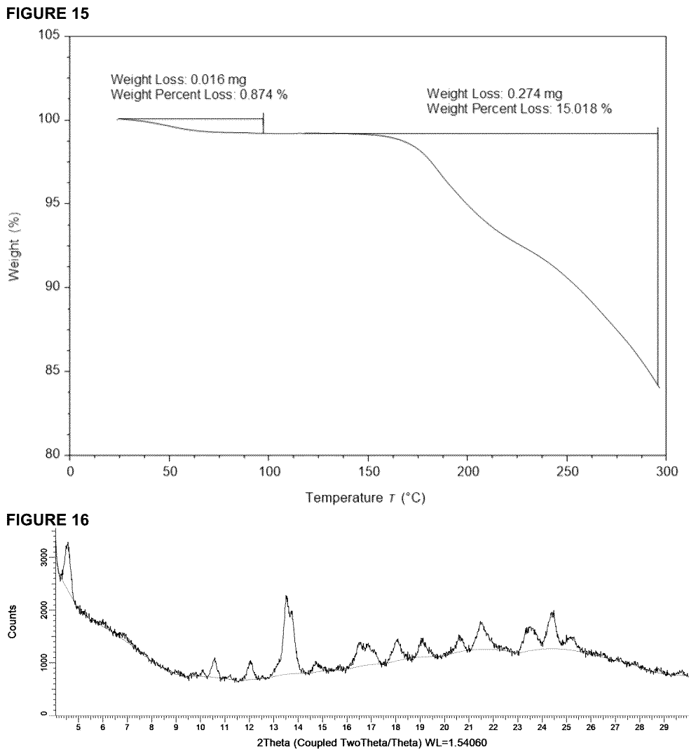

In an embodiment, the compound or pharmaceutically acceptable salt thereof has a DSC thermogram characterized by an endotherm substantially as depicted in FIG. 14. In another embodiment, compound or pharmaceutically acceptable salt thereof has a DSC thermogram characterized by an endotherm having at least one peak at about 154° C., 189° C., or 199° C. In another embodiment, compound or pharmaceutically acceptable salt thereof has a DSC thermogram characterized by an endotherm having an onset at about 26° C. In another embodiment, the compound or pharmaceutically acceptable salt thereof has a DSC thermogram characterized by a dehydration event with an onset temperature of about 26° C. In another embodiment, compound or pharmaceutically acceptable salt thereof has a TGA thermogram substantially as depicted in FIG. 15.

### Compound 1 Phosphate

In an embodiment, the compound or pharmaceutically acceptable salt thereof is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile phosphate.

In another embodiment, the compound or pharmaceutically acceptable salt thereof is characterized by an XRPD diffractogram having at least one (or two) of the following peaks expressed in degrees-2-theta at angles (±0.2 degrees) of 4.5, 13.5, 13.7, 16.5, 16.9, 18.1, 21.5, 23.5, and 24.4.

In yet another embodiment, the compound or pharmaceutically acceptable salt thereof is characterized by an XRPD diffractogram having at least three (or four, five, six, seven, eight, or all) of the following peaks expressed in degrees-2-theta at angles (±0.2 degrees) of 4.5, 13.5, 13.7, 16.5, 16.9, 18.1, 21.5, 23.5, and 24.4.

In still another embodiment, the compound or pharmaceutically acceptable salt thereof is characterized by an XRPD diffractogram having peaks expressed in degrees-2-theta at angles (±0.2 degrees) of 4.5, 13.5, and 13.7. In an embodiment, the compound or pharmaceutically acceptable salt thereof is characterized by an XRPD diffractogram having at least six peaks expressed in degrees-2-theta at angles (±0.2 degrees) of 4.5, 13.5, 13.7, 16.5, 16.9, 18.1, 21.5, 23.5, and 24.4.

In another embodiment, the compound or pharmaceutically acceptable salt thereof is characterized by an XRPD diffractogram having peaks expressed in degrees-2-theta at angles (±0.2 degrees) of 4.5, 13.5, 13.7, 16.5, 21.5, and 24.4. In yet another embodiment, the compound or pharmaceutically acceptable salt thereof is characterized by an XRPD diffractogram having at least nine peaks expressed in degrees-2-theta at angles (±0.2 degrees) of 4.5, 13.5, 13.7, 16.5, 16.9, 18.1, 21.5, 23.5, and 24.4.

In still another embodiment, the compound or pharmaceutically acceptable salt thereof is characterized by an XRPD diffractogram having peaks expressed in degrees-2-theta at angles (±0.2 degrees) of 4.5, 13.5, 13.7, 16.5, 16.9, 18.1, 21.5, 23.5, and 24.4.

In another embodiment, the compound or pharmaceutically acceptable salt thereof is characterized by an XRPD diffractogram substantially as depicted in FIG. 16.

In another embodiment, the compound or pharmaceutically acceptable salt thereof is characterized by an XRPD diffractogram having peaks expressed in degrees-2-theta at angles (±0.2 degrees) selected from the list in Table 6.

TABLE 6

| 2-Theta (°) | H % |
|---|---|
| 4.5 | 59.3 |
| 6.9 | 9.3 |
| 10.1 | 9.2 |
| 10.6 | 25.3 |
| 11.2 | 4.8 |
| 12.0 | 21.7 |
| 13.5 | 100 |
| 13.7 | 79.8 |
| 14.7 | 14.6 |
| 16.5 | 32.0 |
| 16.9 | 28.6 |
| 17.1 | 17.6 |
| 18.3 | 8.1 |
| 18.1 | 28.2 |
| 19.1 | 22.9 |
| 20.6 | 18.5 |
| 21.5 | 36.3 |
| 23.5 | 32.1 |
| 24.4 | 48.2 |
| 25.2 | 17.5 |
| 28.7 | 5.5 |

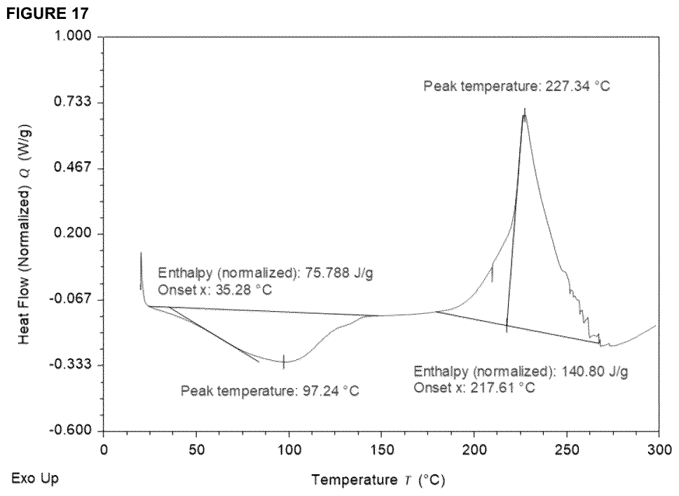

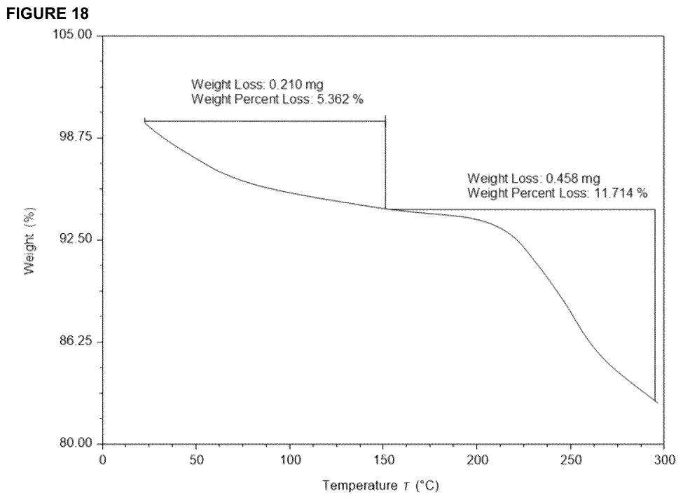

In another embodiment, the compound or pharmaceutically acceptable salt thereof has a DSC thermogram characterized by an endotherm substantially as depicted in FIG. 17. In yet another embodiment, the compound or pharmaceutically acceptable salt thereof has a DSC thermogram characterized by an exotherm having a peak at about 227° C. In yet another embodiment, the compound or pharmaceutically acceptable salt thereof has a DSC thermogram characterized by an exotherm having an onset at about 218° C. In another embodiment, the compound or pharmaceutically acceptable salt thereof has a DSC thermogram characterized by a dehydration event below about 150° C. In still another embodiment, the compound or pharmaceutically acceptable salt thereof has a TGA thermogram substantially as depicted in FIG. 18.

### Compound 1 Free Base

In an embodiment, the compound or pharmaceutically acceptable salt thereof is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

In another embodiment, the compound or pharmaceutically acceptable salt thereof is characterized by an XRPD diffractogram having at least one (or two) of the following peaks expressed in degrees-2-theta at angles (±0.2 degrees) of 5.7, 7.1, 8.3, 13.0, 13.5, 14.3, 15.5, 18.7, 20.8, and 22.7.

In yet another embodiment, the compound or pharmaceutically acceptable salt thereof is characterized by an XRPD diffractogram having at least three (or four, five, six, seven, eight, nine, or all) of the following peaks in degrees-2-theta at angles (±0.2 degrees) of 5.7, 7.1, 8.3, 13.0, 13.5, 14.3, 15.5, 18.7, 20.8, and 22.7.

In still another embodiment, the compound or pharmaceutically acceptable salt thereof is characterized by an XRPD diffractogram having peaks expressed in degrees-2-theta at angles (±0.2 degrees) of 5.7, 7.1, and 8.3. In an embodiment, the compound or pharmaceutically acceptable salt thereof is characterized by an XRPD diffractogram having at least six peaks expressed in degrees-2-theta at angles (±0.2 degrees) of 5.7, 7.1, 8.3, 13.0, 13.5, 14.3, 15.5, 18.7, 20.8, and 22.7.

In another embodiment, the compound or pharmaceutically acceptable salt thereof is characterized by an XRPD diffractogram having peaks expressed in degrees-2-theta at angles (±0.2 degrees) of 5.7, 7.1, 8.3, 13.0, 15.5, and 18.7. In yet another embodiment, the compound or pharmaceutically acceptable salt thereof is characterized by an XRPD diffractogram having at least nine peaks expressed in degrees-2-theta at angles (±0.2 degrees) of 5.7, 7.1, 8.3, 13.0, 13.5, 14.3, 15.5, 18.7, 20.8, and 22.7.

In still another embodiment, the compound or pharmaceutically acceptable salt thereof is characterized by an XRPD diffractogram having peaks expressed in degrees-2-theta at angles (±0.2 degrees) of 5.7, 7.1, 8.3, 13.0, 13.5, 14.3, 15.5, 18.7, 20.8, and 22.7.

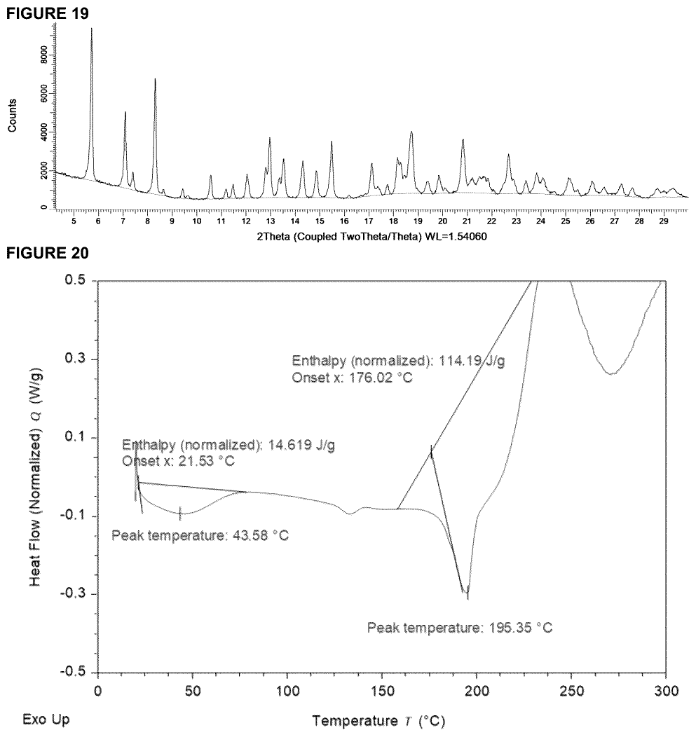

In another embodiment, the compound or pharmaceutically acceptable salt thereof is characterized by an XRPD diffractogram substantially as depicted in FIG. 19.

In another embodiment, the compound or pharmaceutically acceptable salt thereof is characterized by an XRPD diffractogram having peaks expressed in degrees-2-theta at angles (±0.2 degrees) selected from the list in Table 7.

TABLE 7

| 2-Theta (°) | H % |
|---|---|
| 5.7 | 100 |
| 7.1 | 50.4 |
| 7.4 | 11.7 |
| 8.3 | 76.4 |
| 8.6 | 4.2 |
| 9.4 | 6.3 |
| 9.6 | 1.8 |
| 10.6 | 16.0 |
| 11.2 | 6.5 |
| 11.5 | 9.2 |
| 12.0 | 15.6 |
| 12.8 | 19.8 |
| 13.0 | 39.7 |
| 13.4 | 12.6 |
| 13.5 | 25.6 |
| 14.3 | 24.0 |
| 14.9 | 17.5 |
| 15.5 | 37.5 |
| 16.2 | 2.0 |
| 17.1 | 21.1 |
| 17.4 | 5.7 |
| 17.7 | 6.1 |
| 18.2 | 19.3 |
| 18.7 | 41.3 |
| 19.4 | 8.0 |
| 19.8 | 11.9 |
| 20.1 | 3.5 |
| 20.8 | 35.3 |
| 21.2 | 9.1 |
| 21.5 | 10.6 |
| 21.6 | 10.8 |
| 21.8 | 10.0 |
| 22.7 | 26.4 |
| 22.8 | 9.8 |
| 23.4 | 8.5 |
| 23.8 | 13.8 |
| 24.1 | 10.9 |
| 24.6 | 2.2 |
| 25.2 | 10.6 |
| 25.5 | 3.9 |
| 26.1 | 9.4 |
| 26.6 | 5.4 |
| 27.3 | 7.7 |
| 27.7 | 5.7 |
| 28.7 | 5.7 |
| 29.0 | 4.4 |
| 29.4 | 5.9 |
| 29.6 | 1.2 |

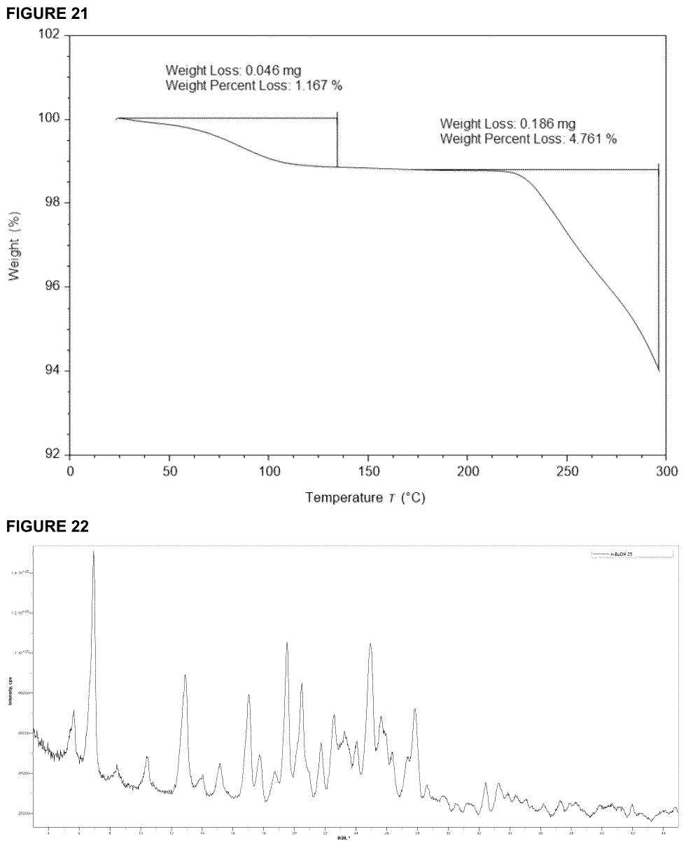

In an embodiment, the compound or pharmaceutically acceptable salt thereof has a DSC thermogram characterized by an endotherm substantially as depicted in FIG. 20. In another embodiment, the compound or pharmaceutically acceptable salt thereof has a DSC thermogram characterized by an endotherm having a peak at about 195° C. In another embodiment, the compound or pharmaceutically acceptable salt thereof has a DSC thermogram characterized by an endotherm having an onset at about 176° C. In another embodiment, the compound or pharmaceutically acceptable salt thereof has a DSC thermogram characterized by a dehydration event below about 100° C. In yet another embodiment, the compound or pharmaceutically acceptable salt thereof has a TGA thermogram substantially as depicted in FIG. 21.

### III. Methods of Treatment

Compound 1 of the present disclosure can inhibit the activity of the KRAS protein, particularly KRAS protein harboring a G12D mutation. Therefore Compound 1, its crystalline forms, its crystalline salts and crystalline forms thereof, and formulations and dosage forms thereof as described in the present disclosure (collectively referred to as “compositions of the disclosure”) can be used to inhibit activity of KRAS, including KRAS harboring G12D, in a cell or in an individual or subject in need of inhibition of the enzyme by administering an inhibiting amount of the compound to the cell, individual, or subject. Compositions of the disclosure are therefore useful in treating diseases including cancers, in which KRAS particularly KRAS harboring a G12D mutation is implicated. Diseases in which KRAS is implicated include diseases associated with the expression or activity of KRAS, e.g., diseases in which abnormally proliferating cells (e.g., of a cancer) express KRAS. Diseases in which KRAS harboring a G12D mutation is implicated include diseases associated with the expression or activity of KRAS harboring a G12D mutation, e.g., diseases in which abnormally proliferating cells (e.g., of a cancer) express or comprise KRAS harboring a G12D mutation.

The cancer types in which KRAS, particularly KRAS harboring a G12D mutation, is implicated, and which can be treated using the compositions of the disclosure, include, but are not limited to: carcinomas (e.g., pancreatic, colorectal, lung, bladder, gastric, esophageal, breast, head and neck, cervical skin, thyroid); hematopoietic malignancies (e.g., myeloproliferative neoplasms (MPN), myelodysplastic syndrome (MDS), chronic and juvenile myelomonocytic leukemia (CMML and JMML), acute myeloid leukemia (AML), acute lymphocytic leukemia (ALL) and multiple myeloma (MM)); and other neoplasms (e.g., glioblastoma and sarcomas). In addition, KRAS mutations were found in acquired resistance to anti-EGFR therapy (Knickelbein, K. et al. Genes & Cancer, (2015): 4-12). KRAS mutations were found in immunological and inflammatory disorders (Fernandez-Medarde, A. et al. Genes & Cancer, (2011): 344-358) such as Ras-associated lymphoproliferative disorder (RALD) or juvenile myelomonocytic leukemia (JMML) caused by somatic mutations of KRAS or NRAS.

Compositions of the disclosure are useful in the treatment of various diseases associated with abnormal expression or activity of KRAS. Compounds which inhibit KRAS will be useful in providing a means of preventing the growth or inducing apoptosis in tumors, or by inhibiting angiogenesis. It is therefore anticipated that compositions of the disclosure will prove useful in treating or preventing proliferative disorders such as cancers. In particular, tumors with activating mutants of receptor tyrosine kinases or upregulation of receptor tyrosine kinases may be particularly sensitive to the inhibitors.

In an aspect, provided herein is a method of inhibiting KRAS activity, said method comprising contacting Compound 1 with KRAS. In an embodiment, the contacting comprises administering a composition of the disclosure to a subject.

In an aspect, provided herein is a method of inhibiting a KRAS protein harboring a G12D mutation, said method comprising contacting KRAS with a Compound 1 In an embodiment, the contacting comprises administering a composition of the disclosure to a subject.

Thus, in an aspect, provided herein is a method of treating cancer in a subject in need thereof comprising administering to the subject a therapeutically effective amount of Compound 1, or a pharmaceutically acceptable salt thereof in the form of a composition of the disclosure. In an aspect, the cancer can be a cancer in which KRAS harboring a G12D mutation is implicated, such as cancers associated with the expression or activity of KRAS harboring a G12D mutation. Such cancers can include cancers in which abnormally proliferating cells (e.g., of a cancer) express or comprise KRAS harboring a G12D mutation.

In an embodiment, the cancer is associated with expression or activity of a KRAS protein. In another embodiment, the cancer is associated with expression or activity of a KRAS protein having a G12D mutation.

In another aspect, provided herein is a method for treating a cancer in a subject comprising identifying that the subject is in need of treatment of a cancer and that abnormally proliferating cells of the cancer comprise KRAS having a G12D mutation, and administering to the subject a therapeutically effective amount of Compound 1, or a pharmaceutically acceptable salt thereof in the form of a composition of the disclosure.

In an embodiment, the cancer is colorectal cancer. In another embodiment, the cancer is pancreatic cancer. In yet another embodiment, the cancer is pancreatic ductal cancer. In still another embodiment, the cancer is lung cancer. In another embodiment, the cancer is non-small cell lung cancer (NSCLC).

In another embodiment, the cancer is metastatic.

In still another embodiment, abnormally proliferating cells of the cancer comprise KRAS having a G12D mutation.

In another aspect, provided herein a is method of treating a disease or disorder associated with activity of KRAS, said method comprising administering to a subject in need thereof a therapeutically effective amount of a composition of the disclosure.

In an embodiment, the disease or disorder is an immunological or inflammatory disorder. In another embodiment, the immunological or inflammatory disorder is Ras-associated lymphoproliferative disorder or juvenile myelomonocytic leukemia caused by somatic mutations of KRAS.

In yet another aspect, provided herein is a method of treating a disease or disorder associated with abnormal activity of a KRAS protein harboring a G12D mutation, said method comprising administering to a subject in need thereof a therapeutically effective amount of a composition of the disclosure.

In another aspect, provided herein is a method of treating a disease or disorder associated with abnormal activity of a KRAS protein harboring a G12V mutation, said method comprising administering to a subject in need thereof a therapeutically effective amount of a composition of the disclosure.

In another aspect, provided herein is also a method of treating cancer in a subject in need thereof comprising administering to the subject a therapeutically effective amount of composition of the disclosure.

In still another aspect, provided herein is also a method of treating cancer in a subject in need thereof comprising administering to the subject a therapeutically effective amount of a composition of the disclosure wherein the cancer is characterized by with the presence or activity of a KRAS protein harboring a G12D mutation.

In yet another aspect, provided herein is a method for treating a cancer in a subject, said method comprising administering to the subject a therapeutically effective amount of composition of the disclosure.

In an embodiment, the cancer is selected from carcinomas, hematological cancers, sarcomas, and glioblastoma. In another embodiment, the hematological cancer is selected from myeloproliferative neoplasms, myelodysplastic syndrome, chronic and juvenile myelomonocytic leukemia, acute myeloid leukemia, acute lymphocytic leukemia, and multiple myeloma. In yet another embodiment, the carcinoma is selected from pancreatic, colorectal, lung, bladder, gastric, esophageal, breast, head and neck, cervical, skin, and thyroid.

In an aspect, provided herein is a method for treating a disease or disorder associated with activity of KRAS interaction or a mutant thereof, in a subject in need thereof, comprising the step of administering to the subject a composition of the disclosure, in combination with another therapy or therapeutic agent as described herein.

In an embodiment, the cancer is selected from hematological cancers, sarcomas, lung cancers, gastrointestinal cancers, genitourinary tract cancers, liver cancers, bone cancers, nervous system cancers, gynecological cancers, and skin cancers.

In another embodiment, the lung cancer is selected from non-small cell lung cancer (NSCLC), small cell lung cancer, bronchogenic carcinoma, squamous cell bronchogenic carcinoma, undifferentiated small cell bronchogenic carcinoma, undifferentiated large cell bronchogenic carcinoma, adenocarcinoma, bronchogenic carcinoma, alveolar carcinoma, bronchiolar carcinoma, bronchial adenoma, chondromatous hamartoma, mesothelioma, pavicellular and non-pavicellular carcinoma, bronchial adenoma, and pleuropulmonary blastoma.

In yet another embodiment, the lung cancer is non-small cell lung cancer (NSCLC). In still another embodiment, the lung cancer is adenocarcinoma.

In an embodiment, the gastrointestinal cancer is selected from esophagus squamous cell carcinoma, esophagus adenocarcinoma, esophagus leiomyosarcoma, esophagus lymphoma, stomach carcinoma, stomach lymphoma, stomach leiomyosarcoma, exocrine pancreatic carcinoma, pancreatic ductal adenocarcinoma, pancreatic insulinoma, pancreatic glucagonoma, pancreatic gastrinoma, pancreatic carcinoid tumors, pancreatic vipoma, small bowel adenocarcinoma, small bowel lymphoma, small bowel carcinoid tumors, Kaposi's sarcoma, small bowel leiomyoma, small bowel hemangioma, small bowel lipoma, small bowel neurofibroma, small bowel fibroma, large bowel adenocarcinoma, large bowel tubular adenoma, large bowel villous adenoma, large bowel hamartoma, large bowel leiomyoma, colorectal cancer, gall bladder cancer, and anal cancer.

In an embodiment, the gastrointestinal cancer is colorectal cancer.

In another embodiment, the cancer is a carcinoma. In yet another embodiment, the carcinoma is selected from pancreatic carcinoma, colorectal carcinoma, lung carcinoma, bladder carcinoma, gastric carcinoma, esophageal carcinoma, breast carcinoma, head and neck carcinoma, cervical skin carcinoma, and thyroid carcinoma.

In still another embodiment, the cancer is a hematopoietic malignancy. In an embodiment, the hematopoietic malignancy is selected from multiple myeloma, acute myelogenous leukemia, and myeloproliferative neoplasms.

In another embodiment, the cancer is a neoplasm. In yet another embodiment, the neoplasm is glioblastoma or sarcomas.

In certain embodiments, the disclosure provides a method for treating a KRAS-mediated disorder in a subject in need thereof, comprising the step of administering to said subject a composition of the disclosure, or a pharmaceutically acceptable composition thereof.

In some embodiments, diseases and indications that are treatable using the compositions of the disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), essential thrombocytosis (ET), 8p11 myeloproliferative syndrome, myelodysplasia syndrome (MDS), T-cell acute lymphoblastic lymphoma (T-ALL), multiple myeloma, cutaneous T-cell lymphoma, adult T-cell leukemia, Waldenstrom's Macroglubulinemia, hairy cell lymphoma, marginal zone lymphoma, chronic myelogenic lymphoma and Burkitt's lymphoma. Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, lymphosarcoma, leiomyosarcoma, and teratoma.

Exemplary lung cancers include non-small cell lung cancer (NSCLC), small cell lung cancer, bronchogenic carcinoma (squamous cell, undifferentiated small cell, undifferentiated large cell, adenocarcinoma), alveolar (bronchiolar) carcinoma, bronchial adenoma, chondromatous hamartoma, mesothelioma, pavicellular and non-pavicellular carcinoma, bronchial adenoma and pleuropulmonary blastoma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (exocrine pancreatic carcinoma, ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), colorectal cancer, gall bladder cancer and anal cancer.

Exemplary genitourinary tract cancers include cancers of the kidney (adenocarcinoma, Wilm's tumor [nephroblastoma], renal cell carcinoma), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma) and urothelial carcinoma.

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, meduoblastoma, glioma, ependymoma, germinoma (pinealoma), glioblastoma, glioblastoma multiform, oligodendroglioma, schwannoma, retinoblastoma, congenital tumors, neuro-ectodermal tumors), and spinal cord (neurofibroma, meningioma, glioma, sarcoma), neuroblastoma, Lhermitte-Duclos disease and pineal tumors.

Exemplary gynecological cancers include cancers of the breast (ductal carcinoma, lobular carcinoma, breast sarcoma, triple-negative breast cancer, HER2-positive breast cancer, inflammatory breast cancer, papillary carcinoma), uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, squamous cell carcinoma, Kaposi's sarcoma, Merkel cell skin cancer, moles dysplastic nevi, lipoma, angioma, dermatofibroma, and keloids.

Exemplary head and neck cancers include glioblastoma, melanoma, rhabdosarcoma, lymphosarcoma, osteosarcoma, squamous cell carcinomas, adenocarcinomas, oral cancer, laryngeal cancer, nasopharyngeal cancer, nasal and paranasal cancers, thyroid and parathyroid cancers, tumors of the eye, tumors of the lips and mouth and squamous head and neck cancer.

The compositions of the disclosure can also be useful in the inhibition of tumor metastases.

In addition to oncogenic neoplasms, the compositions of the disclosure are useful in the treatment of skeletal and chondrocyte disorders including, but not limited to, achrondroplasia, hypochondroplasia, dwarfism, thanatophoric dysplasia (TD) (clinical forms TD I and TD II), Apert syndrome, Crouzon syndrome, Jackson-Weiss syndrome, Beare-Stevenson cutis gyrate syndrome, Pfeiffer syndrome, and craniosynostosis syndromes. In some embodiments, the present disclosure provides a method for treating a subject suffering from a skeletal and chondrocyte disorder.

In an embodiment, the cancer is selected from hematological cancers, sarcomas, lung cancers, gastrointestinal cancers, genitourinary tract cancers, liver cancers, bone cancers, nervous system cancers, gynecological cancers, and skin cancers.

In another embodiment, the lung cancer is selected from non-small cell lung cancer (NSCLC), small cell lung cancer, bronchogenic carcinoma, squamous cell bronchogenic carcinoma, undifferentiated small cell bronchogenic carcinoma, undifferentiated large cell bronchogenic carcinoma, adenocarcinoma, bronchogenic carcinoma, alveolar carcinoma, bronchiolar carcinoma, bronchial adenoma, chondromatous hamartoma, mesothelioma, pavicellular and non-pavicellular carcinoma, bronchial adenoma, and pleuropulmonary blastoma.

In yet another embodiment, the lung cancer is non-small cell lung cancer (NSCLC). In still another embodiment, the lung cancer is adenocarcinoma.

In an embodiment, the gastrointestinal cancer is selected from esophagus squamous cell carcinoma, esophagus adenocarcinoma, esophagus leiomyosarcoma, esophagus lymphoma, stomach carcinoma, stomach lymphoma, stomach leiomyosarcoma, exocrine pancreatic carcinoma, pancreatic ductal adenocarcinoma, pancreatic insulinoma, pancreatic glucagonoma, pancreatic gastrinoma, pancreatic carcinoid tumors, pancreatic vipoma, small bowel adenocarcinoma, small bowel lymphoma, small bowel carcinoid tumors, Kaposi's sarcoma, small bowel leiomyoma, small bowel hemangioma, small bowel lipoma, small bowel neurofibroma, small bowel fibroma, large bowel adenocarcinoma, large bowel tubular adenoma, large bowel villous adenoma, large bowel hamartoma, large bowel leiomyoma, colorectal cancer, gall bladder cancer, and anal cancer.

In an embodiment, the gastrointestinal cancer is colorectal cancer.

In another embodiment, the cancer is a carcinoma. In yet another embodiment, the carcinoma is selected from pancreatic carcinoma, colorectal carcinoma, lung carcinoma, bladder carcinoma, gastric carcinoma, esophageal carcinoma, breast carcinoma, head and neck carcinoma, cervical skin carcinoma, and thyroid carcinoma.

In still another embodiment, the cancer is a hematopoietic malignancy. In an embodiment, the hematopoietic malignancy is selected from multiple myeloma, acute myelogenous leukemia, and myeloproliferative neoplasms.

In another embodiment, the cancer is a neoplasm. In yet another embodiment, the neoplasm is glioblastoma or sarcomas.

In an embodiment, the cancer is selected from the group consisting of hematological cancers, sarcomas, lung cancers, gastrointestinal cancers, genitourinary tract cancers, liver cancers, bone cancers, nervous system cancers, gynecological cancers, and skin cancers.

In an embodiment, the cancer is selected from the group consisting of pancreatic cancer, cervical cancer, colon cancer, ovarian cancer, breast cancer, pancreatic cancer, carcinoma, and adenocarcinoma.

In another embodiment, the cancer is pancreatic cancer. In yet another embodiment, the cancer is a solid tumor.

In one embodiment of the methods described herein, the subject is human.

### IV. Pharmaceutical Compositions

Compound 1, its crystalline forms, its crystalline salts and crystalline forms thereof, as described in the present disclosure can be administered in pharmaceutical compositions comprising a crystalline form disclosed herein and at least one pharmaceutically acceptable carrier or excipient.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water soluble compounds such as those used for vitamin K. For enteral administration, the compound may be administered in a tablet, capsule or dissolved in liquid form. The tablet or capsule may be enteric coated, or in a formulation for sustained release. Many suitable formulations are known, including, polymeric or protein microparticles encapsulating a compound to be released, ointments, pastes, gels, hydrogels, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Lippencott Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful parenteral delivery systems for modulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

In an aspect, provided herein is a pharmaceutical composition comprising:
a) compound 1, its crystalline forms, its crystalline salts and crystalline forms thereof, and formulations and dosage forms thereof as described in the present disclosure;
b) a disintegrant;
c) a binder;
d) an anti-caking agent; and
e) a lubricant.

In an embodiment, the composition comprises two lubricants. In an embodiment, the composition further comprises f) an additive. In an embodiment, the composition further comprises f) a second lubricant.

In another embodiment, the crystalline form of a) is present in a dose of between about 25 mg and 200 mg. In yet another embodiment, the crystalline form of a) is present in a dose of about 25 mg. In still another embodiment, the crystalline form of a) is present in a dose of about 100 mg. In an embodiment, the crystalline form of a) is present in a dose of about 200 mg.

In another embodiment, disintegrant b) is sodium starch glycolate.

In yet another embodiment, binder c) is MCC PH102. In another embodiment, binder c) is HPMC (Methocel E5 Premium LV Hydroxypropyl Methylcellulose).

In still another embodiment, anti-caking agent d) is colloidal silicon dioxide.

In an embodiment, lubricant e) is sodium stearyl fumarate.

In another embodiment, additive f) is magnesium stearate. In another embodiment, second lubricant f) is magnesium stearate.

In another aspect, provided herein is a pharmaceutical composition comprising:
a) the crystalline form of the present disclosure;
b) sodium starch glycolate;
c) MCC PH102;
d) colloidal silicon dioxide;
e) sodium stearyl fumarate; and
f) magnesium stearate.

In an embodiment, the pharmaceutical composition is a tablet.

### IV. Processes for Preparing

The present disclosure provides methods for preparing salts of the present disclosure.

Thus, in an aspect, provided herein is a process for preparing 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate comprising reacting 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile with aqueous hydrochloric acid.

In an embodiment, the reacting is performed in a solvent comprising an alcohol. In another embodiment, the reacting is performed in a solvent comprising a dialkyl ether. In yet another embodiment, the reacting is performed in a solvent comprising an alkanoic acid ester. In still another embodiment, the reacting is performed in a solvent comprising an alcohol, a dialkyl ether and an alkanoic acid alkyl ester.

In an embodiment, the alcohol is methanol. In another embodiment, the dialkyl ether is methyl tert-butyl ether. In another embodiment, the alkanoic acid alkyl ester is ethyl acetate.

In yet another embodiment, the 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile and hydrochloric acid are reacted in a molar ratio of substantially 1:1.

In still another embodiment, the 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

In another embodiment, the 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile is 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

### IV. Administration/Dosage/Formulations

In another aspect, provided herein is a pharmaceutical composition comprising a crystalline form provided herein, together with a pharmaceutically acceptable carrier.

Actual dosage levels of the active ingredients in the pharmaceutical compositions discussed herein may be varied to obtain an amount of the active ingredient that is effective to achieve the desired therapeutic response for a particular subject, composition, and mode of administration, without being toxic to the subject.

In particular, the selected dosage level will depend upon a variety of factors including the activity of the particular compound employed, the time of administration, the rate of excretion of the compound, the duration of the treatment, other drugs, compounds or materials used in combination with the crystalline form, the age, sex, weight, condition, general health and prior medical history of the subject being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed crystalline form at levels lower than that required in order to achieve the desired therapeutic effect and gradually increase the dosage until the desired effect is achieved.

In particular embodiments, it is especially advantageous to formulate the crystalline form in dosage unit form for ease of administration and uniformity of dosage.

“Dosage unit form,” as used herein, refers to physically discrete units suited as unitary dosages for the subjects to be treated; each unit containing a predetermined quantity of the disclosed compound calculated to produce the desired therapeutic effect in association with the required pharmaceutical vehicle. The dosage unit forms of the crystalline form disclosed herein are dictated by and directly dependent on (a) the unique characteristics of the disclosed compound and the particular therapeutic effect to be achieved, and (b) the limitations inherent in the art of compounding/formulating such a disclosed compound for the treatment of cancer in a subject.

As used herein, the term “free base equivalent” refers to the amount of the active agent (e.g., Compound 1) present in the active agent or pharmaceutically acceptable salt thereof. Stated alternatively, the term “free base equivalent” means either an amount of Compound 1 free base, or the equivalent amount of Compound 1 free base that is provided by a salt of said compound. The dosages provided herein refer to the free base equivalent of Compound 1.

In one embodiment, the crystalline form provided herein is formulated using one or more pharmaceutically acceptable excipients or carriers. In one embodiment, the pharmaceutical compositions comprise a therapeutically effective amount of the disclosed crystalline form and a pharmaceutically acceptable carrier.

In an aspect, provided herein is a dosage form comprising Compound 1 or pharmaceutically acceptable salt thereof or a pharmaceutical composition disclosed herein.

In an embodiment, the dosage form is in the form of a tablet. In another embodiment, the compound or pharmaceutically acceptable salt thereof is present in an amount that provides a dose of about 25 mg to about 200 mg of 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile per dosage form.

In another embodiment, the compound or pharmaceutically acceptable salt thereof is present in an amount that provides a dose of about 25 mg of 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile per dosage form.

In yet another embodiment, the compound or pharmaceutically acceptable salt thereof a) is present in an amount that provides a dose of about 100 mg of 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile per dosage form.

In still another embodiment, the compound or pharmaceutically acceptable salt thereof a) is present in an amount that provides a dose of about 200 mg of 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile per dosage form.

In some embodiments, the dose of a disclosed crystalline form is from about 1 mg to about 1,000 mg. In some embodiments, the amount of Compound 1, its crystalline forms, its crystalline salts and crystalline forms thereof, and formulations and dosage forms thereof as described in the present disclosure are included in an amount that provides a dose Compound 1 (calculated as the amount equivalent to the amount of free base of Compound 1) that is less than about 1,000 mg, or less than about 800 mg, or less than about 600 mg, or less than about 500 mg, or less than about 300 mg, or less than about 200 mg, or less than about 100 mg, or less than about 50 mg, or less than about 20 mg, or less than about 10 mg. For example, a dose is about 10 mg, 20 mg, 25 mg, 30 mg, 40 mg, 50 mg, 60 mg, 70 mg, 80 mg, 90 mg, 100 mg, 120 mg, 140 mg, 160 mg, 180 mg, 200 mg, 220 mg, 240, 260 mg, 280 mg, 300 mg, 350 mg, 400 mg, 450 mg, 500 mg, 550 mg, or about 600 mg.

Routes of administration of any of the compositions disclosed herein include oral, nasal, rectal, intravaginal, parenteral, buccal, sublingual or topical. The compound for use provided herein may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example, an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compound may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The disclosure is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

The KRAS inhibitor provided herein, its synthesis and its biological activity against KRAS can be found in WO 2023/064857, which is incorporated by reference in its entirety.

### Example 1: Synthesis of 3-(1-((1R,4R,5S)-2-Azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile (Compound 1)

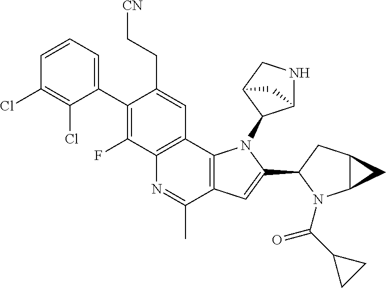 [SMILES: Cc1nc2c(F)c(-c3cccc(Cl)c3Cl)c(CCC#N)cc2c2c1cc([C@H]1C[C@H]3C[C@H]3N1C(=O)C1CC1)n2[C@H]1[C@H]2CN[C@@H]1C2]

### Step 1. Methyl 2-amino-4-bromo-3-fluorobenzoate

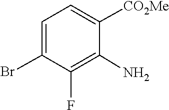 [SMILES: COC(=O)c1ccc(Br)c(F)c1N]

Dimethyl sulfate (823 g, 6.53 mole) was added to a mixture of 2-amino-4-bromo-3-fluorobenzoic acid (1500 g, 6.22 mole) and potassium carbonate (945 g, 6.84 mole) in N,N-dimethylamide or 1,4-dioxane (6 L) at 5-50° C. After the addition, the mixture was stirred at room temperature for 2 hours to complete the reaction. Water (7.5 L) was gradually added to the reaction mixture to precipitate the product. After the water addition, the mixture was stirred at room temperature for 1 hour. The solids were isolated by filtration and the wet cake was washed with water (3×1.5 L). The solids were dried under vacuum at about 50° C. overnight to give desired product (1530 g, 99% yield). LCMS calculated for C₈H₇BrFNO₂: 246.96; Found: 248 (M+H⁺). ¹H NMR (400 MHz, DMSO-d₆) δ 7.49 (dd, J=8.8, 1.7 Hz, 1H), 6.87-6.77 (m, 3H), 3.82 (s, 3H). ¹⁹F NMR (376 MHz, DMSO-d₆) δ −127.24

### Step 2. Methyl 3-amino-2′,3′-dichloro-2-fluoro-[1,1′-biphenyl]-4-carboxylate

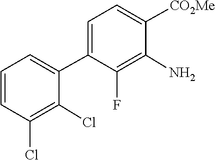 [SMILES: COC(=O)c1ccc(-c2cccc(Cl)c2Cl)c(F)c1N]

Bis(di-tert-butyl(4-dimethylaminophenyl)phosphine)dichloropalladium (II) (Pd-132) (8.12 g, 0.011 mole) was added to a mixture of methyl 2-amino-4-bromo-3-fluorobenzoate (1420 g, 5.72 mole), 2,3-dichlorophenylboronic acid (1226 g, 6.3 mole) and potassium fluoride (732 g, 12.6 mole) in acetonitrile (6 L) and water (1.5 L). The mixture was degassed and refilled with nitrogen and heated to 70° C. for 1 hour to complete the reaction. Water (6 L) was added to the reaction mixture at 50° C. The mixture was cooled to room temperature and stirred for 1 hour. The solids were isolated by filtration and the wet cake was washed with 50% acetonitrile in water (2×2 L) and water (2×2 L). The solids were dried under vacuum at about 50° C. overnight to give desired product (1700 g, 94% yield). LCMS calculated for C₁₄H₉Cl₂FNO₂: 313.01; Found: 314 (M+H⁺). ¹H NMR (400 MHz, DMSO-d₆) δ 7.74 (dd, J=8.0, 1.6 Hz, 1H), 7.64 (dd, J=8.4, 1.4 Hz, 1H), 7.48 (t, J=7.9 Hz, 1H), 7.40 (dd, J=7.9, 1.6 Hz, 1H), 6.70 (s (b), 2H), 6.51 (dd, J=8.3, 6.6 Hz, 1H), 3.86 (s, 3H). 19F NMR (376 MHz, DMSO-d₆) δ −134.70

### Step 3. 3-Amino-6-bromo-2′,3′-dichloro-2-fluoro-[1,1′-biphenyl]-4-carboxylic acid

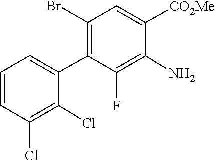 [SMILES: COC(=O)c1cc(Br)c(-c2cccc(Cl)c2Cl)c(F)c1N]

N-Bromosuccinimide (684 g, 3.84 mole) was added to a solution of methyl 3-amino-2′,3′-dichloro-2-fluoro-[1,1′-biphenyl]-4-carboxylate (1150, 3.66 mole) in acetonitrile (5.75 L) at 50-66° C. After the reaction completion, the acetonitrile (3 L) was removed by rotavapor. Water (5.75 L) was added to the concentrated mixture and stirred at room temperature for 2-3 hours. The solids were isolated by filtration and the wet cake was washed with water to give methyl 3-amino-6-bromo-2′,3′-dichloro-2-fluoro-[1,1′-biphenyl]-4-carboxylate. LCMS calculated for C₁₄H₉BrFCl₂NO₂: 390.92; Found: 391 (M+H). ¹H NMR (400 MHz, DMSO-d₆) δ 7.86 (d, J=1.7 Hz, 1H), 7.79 (dd, J=8.1, 1.5 Hz, 1H), 7.52 (t, J=7.9 Hz, 1H), 7.40 (dd, J=7.7, 1.5 Hz, 1H), 6.83 (s(b), 2H), 3.87 (s, 3H). ¹⁹F NMR (376 MHz, DMSO-d₆) δ −128.19.

### Step 4. 3-Amino-6-bromo-2′,3′-dichloro-2-fluoro-[1,1′-biphenyl]-4-carboxylic acid

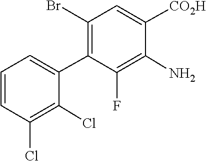 [SMILES: Nc1c(C(=O)O)cc(Br)c(-c2cccc(Cl)c2Cl)c1F]

The wet cake of 3-amino-6-bromo-2′,3′-dichloro-2-fluoro-[1,1′-biphenyl]-4-carboxylic acid was dissolved in THF (3 L) and methanol (1.5 L). Sodium hydroxide (1.5 M) aqueous solution (5 L) was added to the solution and the mixture was stirred at about 50° C. for 2 hours to complete the saponification reaction. Hydrochloric acid (1.5 M) aqueous solution was gradually added to the mixture to adjust the pH to 3-4 and stirred at room temperature for 1 hour. The solids were isolated by filtration and the wet cake was washed with water (3×1.2 L). The solids were dried under vacuum at about 50° C. overnight to give desired product (1354 g, 97.5% yield over two steps). LCMS calculated for C₁₃H₇BrCl₂FNO₂: 376.90; Found: 378 (M+H⁺). ¹H NMR (400 MHz, DMSO-d₆) δ 7.85 (d, J=1.7 Hz, 1H), 7.78 (dd, J=8.1, 1.5 Hz, 1H), 7.52 (t, J=7.9 Hz, 1H), 7.39 (dd, J=7.9, 1.5 Hz, 1H), 6.88. ¹⁹F NMR (376 MHz, DMSO-d₆) δ −128.95.

### Step 5. 6-Bromo-7-(2,3-dichlorophenyl)-8-fluoro-2H-benzo[d][1,3]oxazine-2,4(1H)-dione

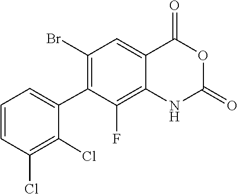 [SMILES: O=c1[nH]c2c(F)c(-c3cccc(Cl)c3Cl)c(Br)cc2c(=O)o1]

Triphosgene (500 g, 1.65 mole) in tetrahydrofuran (THF) (500 mL) was added to the solution of 3-amino-6-bromo-2′,3′-dichloro-2-fluoro-[1,1′-biphenyl]-4-carboxylic acid (1254 g, 3.31 mole) in THF (4 L) at 60° C. and stirred for 1 hour to complete the reaction. The mixture was cooled to 35° C. and n-heptane (10 L) was slowly charged to precipitate the product. The mixture was cooled to room temperature and stirred for 1 hour. The solids were isolated by filtration and washed with n-heptane (2×1 L). The wet cake was dried under vacuum at about 50° C. overnight to give desired product (1385 g, quantitative yield). LCMS calculated for C₁₄H₅BrCl₂FNO₃: 402.88; Found: 404 (M+H⁺). ¹H NMR (400 MHz, DMSO-d₆) δ 12.24 (s, 1H), 8.10 (d, J=1.5 Hz, 1H), 7.85 (dd, J=8.1, 1.5 Hz, 1H), 7.58 (t, J=7.9 Hz, 1H), 7.43 (dd, J=7.7, 1.5 Hz, 1H). ¹⁹F NMR (376 MHz, DMSO-d₆) δ −123.98.

### Step 6. Ethyl 6-bromo-7-(2,3-dichlorophenyl)-8-fluoro-4-hydroxy-2-methylquinoline-3-carboxylate

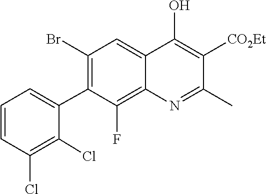 [SMILES: CCOC(=O)c1c(C)nc2c(F)c(-c3cccc(Cl)c3Cl)c(Br)cc2c1O]

A mixture of 6-bromo-7-(2,3-dichlorophenyl)-8-fluoro-2H-benzo[d][1,3]oxazine-2,4(1H)-dione (1078 g, 2.66 mole), ethyl acetoacetate (693 g, 5.32 mole), sodium acetate (393 g, 4.79 mole) and sodium chloride (933 g. 16 mole) in dimethyl sulfoxide (5 L) was heated to 50-60° C. for 5 hours. The temperature was raised to 100° C. and stirred for 1 hour to complete the reaction. The mixture was cooled to about 60° C. and water (10 L) was gradually added to precipitate the product. The mixture was cooled to room temperature and stirred for 1 hour. The solids were isolated by filtration and the wet cake was washed with water (2×2 L). The wet solids were dried under vacuum at about 50° C. overnight to give desired product (1145 g, 91% yield). LCMS calculated for C₁₉H₁₃BrCl₂FNO₂: 470.94; Found: 472 (M+H⁺). ¹H NMR (400 MHz, DMSO-d₆) δ 12.05 (s, 1H), 8.18 (d, J=1.5 Hz, 1H), 7.84 (dd, J=8.0, 1.6 Hz, 1H), 7.58 (t, J=7.9 Hz, 1H), 7.50 (dd, J=7.7, 1.6 Hz, 1H), 4.28 (q, J=7.1 Hz, 2H), 2.46 (s, 3H), 1.29 (t, J=7.1 Hz, 3H). ¹⁹F NMR (376 MHz, DMSO-d₆) δ −124.80.

### Step 6b. Ethyl 6-bromo-7-(2,3-dichlorophenyl)-8-fluoro-4-hydroxy-2-methylquinoline-3-carboxylate

The title compound can alternatively be prepared by the following process. A solution of methyl 3-amino-2′,3′-dichloro-2-fluoro-[1,1′-biphenyl]-4-carboxylate (100 g, 0.254 mole), ethyl acetoacetate (33.1 g, 0.51 mole) and p-toluenesulfonic acid (2.2 g, 0.013 mole) in xylene (1 L) was refluxed for 5 hours to azeotropically remove water. Sodium ethoxide (26 g, 0.381 mole) was added to the mixture and the mixture was refluxed for another 5 hours. The mixture was cooled to room temperature and poured into dilute hydrochloric acid pH=6-7. The organic phase was separated and the aqueous phase was extracted with ethyl acetate. The combined organic phases were concentrated and the product was purified over silica gel column and eluted with ethyl acetate and heptane (0-30%) to give desired product (65 g, 54%). LCMS calculated for C₁₉H₁₃BrCl₂FNO₃: 470.91; Found: 472 (M+H⁺). ¹H NMR (400 MHz, DMSO-d₆) δ 12.05 (s, 1H), 8.18 (d, J=1.5 Hz, 1H), 7.84 (dd, J=8.0, 1.6 Hz, 1H), 7.58 (t, J=7.9 Hz, 1H), 7.50 (dd, J=7.7, 1.6 Hz, 1H), 4.28 (q, J=7.1 Hz, 2H), 2.46 (s, 3H), 1.29 (t, J=7.1 Hz, 3H). ¹⁹F NMR (376 MHz, DMSO-d₆) δ −124.80.

### Step 7. Ethyl 6-(2-cyanovinyl)-7-(2,3-dichlorophenyl)-8-fluoro-4-hydroxy-2-methylquinoline-3-carboxylate

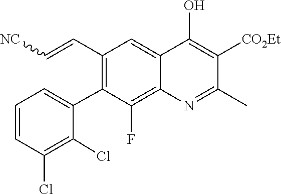 [SMILES: CCOC(=O)c1c(C)nc2c(F)c(-c3cccc(Cl)c3Cl)c(C=CC#N)cc2c1O]

A mixture of ethyl 6-bromo-7-(2,3-dichlorophenyl)-8-fluoro-4-hydroxy-2-methylquinoline-3-carboxylate (246 g, 0.52 mole), acrylonitrile (69 g, 1.3 mole), trimethylamine (156 g, 1.56 mole) and bis(di-tert-butyl)-dimethylaminophenylphosphone dichloride palladium (II) (Pd-132) (14.7 g, 0.02 mole) in N,N-dimethylamide (1.5 L) was heated to 85° C. for about 5 hours to complete the reaction. The mixture was cooled to 50° C. and water (1 L) was gradually added. The mixture was cooled to room temperature and 1M hydrochloric acid aqueous solution was added to adjust the pH to pH 5-6. The solids were isolated by filtration and the wet cake was washed with water (2×500 mL). The wet solids were dissolved in methanol (1 L) and dichloromethane (9 L). To the solution was added sodium bisulfite (186 g, 1.8 mole) and water (4 L). The mixture was stirred at room temperature for 1 hour and the aqueous phase was separated and discarded. The organic phase was washed with water (2×2 L). Activated charcoal (150 g) was added to the organic solution and the mixture was stirred at room temperature for 1 hour. The mixture was filtered over a diatomaceous earth bed and the bed was rinsed with dichloromethane (2 L). The organic solution was concentrated to about 1 L and heptane (3.5 L) was gradually added to precipitate the product. The solids were isolated by filtration and washed with heptane (2×2 L). The wet solids were dried under vacuum at about 50° C. overnight to give desired product (210 g, 90% yield). LCMS calculated for C₂₂H₁₅Cl₂FNO₃: 444.04; Found: 445 (M+H⁺). ¹H-NMR (400 MHz, DMSO-d₆) (cis and trans mixture): 012.05 (s, 1H), 8.64 (s, OH), 8.39 (s, 1H), 7.86 (td, J=7.7, 1.5 Hz, 1H), 7.63-7.53 (m, 1H), 7.47 (td, J=7.5, 1.6 Hz, 1H), 7.04 (d, J=16.5 Hz, 1H), 6.88 (d, J=11.9 Hz, OH), 6.55 (d, J=16.6 Hz, 1H), 5.91 (d, J=12.0 Hz, OH), 4.29 (q, J=7.1 Hz, 2H), 2.47 (d, J=5.0 Hz, 4H), 1.30 (td, J=7.1, 3.2 Hz, 4H).

### Step 8. Ethyl 6-(2-cyanoethyl)-7-(2,3-dichlorophenyl)-8-fluoro-4-hydroxy-2-methylquinoline-3-carboxylate

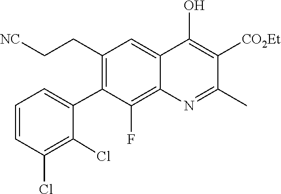 [SMILES: CCOC(=O)c1c(C)nc2c(F)c(-c3cccc(Cl)c3Cl)c(CCC#N)cc2c1O]

A mixture of ethyl 6-(2-cyanovinyl)-7-(2,3-dichlorophenyl)-8-fluoro-4-hydroxyl-2-methylquinoline-3-carboxylate (155 g, 348 mmol), pyridine (450 mL) and 1,4-dioxane (450 mL) was heated to 50-60° C. to give a homogenous solution. To the solution was added sodium borohydride (65.8 g, 1741 mmol) in portions at 50-60° C. The resulting mixture was stirred for 22 hours at 50-60° C. to complete the reduction. After cooling to about 15° C., ethyl acetate (950 mL) was added to the reaction mixture. Concentrated hydrochloric acid was gradually added to the mixture to adjust the aqueous phase pH to 1-2. The organic phase was separated and the aqueous phase was extracted with ethyl acetate (500 mL). The combined ethyl acetate phase was washed with 1N aqueous hydrochloric acid (500 mL), water (2×500 mL), 10% brine (300 mL) and dried over sodium sulfate (75 g). The solution was concentrated and the residue was purified by silica gel column (0-20% MeOH in DCM) to give desired product (117.8 g, 76%). LCMS calculated for C₂₂H₁₇Cl₂FN₂O₃: 446.06; Found: 447 (M+H⁺). ¹H NMR (400 MHz, DMSO-d₆) δ 11.87 (s, 1H), 8.00 (s, 1H), 7.84 (dd, J=7.9, 1.7 Hz, 1H), 7.71-7.48 (m, 2H), 4.28 (q, J=7.1 Hz, 2H), 2.79 (ddd, J=11.7, 7.4, 3.7 Hz, 1H), 2.73-2.59 (m, 3H), 2.46 (s, 3H), 1.30 (t, J=7.1 Hz, 3H).

### Step 9. Ethyl 4-chloro-6-(2-cyanoethyl)-7-(2,3-dichlorophenyl)-8-fluoro-2-methylquinoline-3-carboxylate

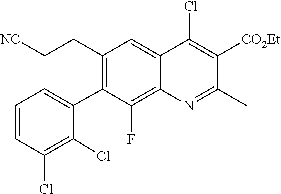 [SMILES: CCOC(=O)c1c(C)nc2c(F)c(-c3cccc(Cl)c3Cl)c(CCC#N)cc2c1Cl]

A mixture of ethyl 6-(2-cyanoethyl)-7-(2,3-dichlorophenyl)-8-fluoro-4-hydroxy-2-methylquinoline-3-carboxylate (60 g, 134 mmol), benzyltriethylammonium chloride (31 g, 135 mmol), N,N-Dimethylaniline (49.1 g, 405 mmol) in acetonitrile (300 mL) was added Phosphorus oxychloride (62 g, 405 mmol) at below 20° C. The mixture was heated to 60° C. for 1 hour to complete the reaction. The mixture was cooled to room temperature and pooled into ice-water (900 mL) at temperature below 20° C. Product precipitated out during the aqueous quench. The mixture was stirred at room temperature for more than 5 hours. The solids were isolated by filtration and the wet cake was washed with 10% acetonitrile in water (2×150 mL). The wet solids were dried under vacuum at about 50° C. overnight to give desired product (57 g, 90% yield). LCMS calculated for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 10. Ethyl 4-chloro-6-(2-cyanovinyl)-7-(2,3-dichlorophenyl)-8-fluoro-2-methylquinoline-3-carboxylate

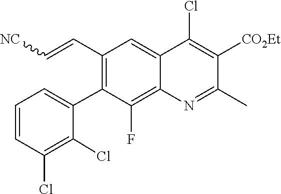 [SMILES: CCOC(=O)c1c(C)nc2c(F)c(-c3cccc(Cl)c3Cl)c(C=CC#N)cc2c1Cl]

A mixture of ethyl 6-(2-cyanovinyl)-7-(2,3-dichlorophenyl)-8-fluoro-4-hydroxy-2-methylquinoline-3-carboxylate (600 g, 1.35 mole), benzyltriethylammonium chloride (307 g, 1.35 mole), N,N-Diethylaniline (603 g, 4.04 mole) in acetonitrile (3 L) was added Phosphorus oxychloride (389.7 g, 4.04 mole) at below 20° C. The mixture was heated to 60° C. for 1 hour to complete the reaction. The mixture was cooled to room temperature and pooled into ice-water (9 L) at temperature below 20° C. Product precipitated out during the aqueous quench. The mixture was stirred at room temperature for more than 5 hours. The solids were isolated by filtration and the wet cake was washed with 10% acetonitrile in water (2×1.5 L). The wet solids were dried under vacuum at about 50° C. overnight to give desired product (563 g, 90% yield). LCMS calculated for C₂₂H₁₄Cl₃FN₂O₂: 462.01; Found: 463 (M+H⁺). ¹H-NMR (400 MHz, DMSO-d₆) (mixture of cis and trans isomers) δ 8.72 (s, 0.3H), 8.51 (s, 1H), 7.87 (ddd, J=7.3, 5.6, 1.5 Hz, 1.3H), 7.64-7.46 (m, 3H), 7.21 (d, J=16.5 Hz, 1H), 7.05 (d, J=11.9 Hz, 0.3H), 6.73 (d, J=16.5 Hz, 1H), 6.08 (d, J=11.9 Hz, 0.3H), 4.53 (qd, J=7.1, 2.0 Hz, 2H), 2.72 (d, J=7.4 Hz, 4H), 1.41 (t, J=7.1 Hz, 4H).

### Step 11. Ethyl 4-chloro-6-(2-cyanoethyl)-7-(2,3-dichlorophenyl)-8-fluoro-2-methylquinoline-3-carboxylate

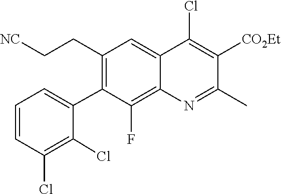 [SMILES: CCOC(=O)c1c(C)nc2c(F)c(-c3cccc(Cl)c3Cl)c(CCC#N)cc2c1Cl]

A mixture of ethyl 4-chloro-6-(2-cyanovinyl)-7-(2,3-dichlorophenyl)-8-fluoro-2-methylquinoline-3-carboxylate (528 g, 1.14 mole) and PMHS (411 g, 6.83 mole) in toluene (1.8 L) were stirred at about 50° C. In another 2-L flask, diacetoxycopper hydrate (4.1 g, 0.02 mole), (9,9-dimethyl-9H-xanthene-4,5-diyl)bis(diphenylphosphane) (13.58 g, 0.023 mol) in toluene (300 ml) and tert-butanol (483 g, 6.52 mole) were stirred for 1-2 hours to a solution. The copper acetate solution was slowly added to the solution of ethyl 4-chloro-6-(2-cyanovinyl)-7-(2,3-dichlorophenyl)-8-fluoro-2-methylquinoline-3-carboxylate and PMHS in toluene at 50-60° C. to complete the reduction. The reaction mixture was concentrated under vacuum distillation to about 2 L. To the 2 L residue was added heptane (8 L) at about 50° C. for 1 hour. The mixture was cooled to room temperature and stirred overnight. The solids were isolated by filtration and the wet cake was washed with heptane (2×1.2 L). The wet cake and silica gel (260 g) in dichloromethane (2.7 L) were stirred for 1 hour. The mixture was filtered over silica gel bed (260 g) and the silica gel bed was rinsed with DCM (4 L) until the eluent was almost colorless. The dichloromethane was removed. Dichloromethane (140 mL) and methyl tert-butyl ether (260 mL) were added to the residue. The solids were isolated by filtration and the wet cake was washed with MTBE (2×1.2 L). The wet solids were dried under vacuum at about 50° C. overnight to give desired product (476 g, 90% yield). LCMS calculated for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H).

### Step 12. Ethyl (Rₐ)-4-chloro-6-(2-cyanoethyl)-7-(2,3-dichlorophenyl)-8-fluoro-2-methylquinoline-3-carboxylate

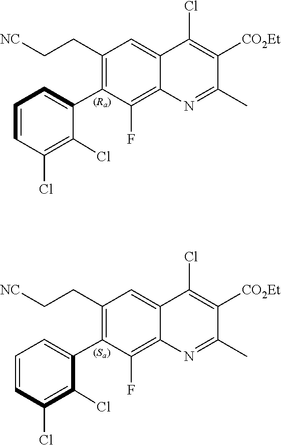

The racemic ethyl 4-chloro-6-(2-cyanoethyl)-7-(2,3-dichlorophenyl)-8-fluoro-2-methylquinoline-3-carboxylate was subject to chiral separation (Chiralpak IB N, MTBE as eluent) to give both ethyl (R)-4-chloro-6-(2-cyanoethyl)-7-(2,3-dichlorophenyl)-8-fluoro-2-methylquinoline-3-carboxylate and ethyl(S)-4-chloro-6-(2-cyanoethyl)-7-(2,3-dichlorophenyl)-8-fluoro-2-methylquinoline-3-carboxylate. LCMS calculated for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H)

### Step 13. Ethyl 4-chloro-6-(2-cyanoethyl)-7-(2,3-dichlorophenyl)-8-fluoro-2-methylquinoline-3-carboxylate by Racemization

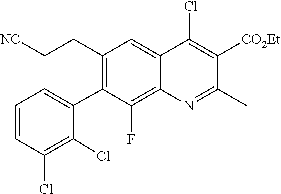 [SMILES: CCOC(=O)c1c(C)nc2c(F)c(-c3cccc(Cl)c3Cl)c(CCC#N)cc2c1Cl]

A mixture of ethyl(S)-4-chloro-6-(2-cyanoethyl)-7-(2,3-dichlorophenyl)-8-fluoro-2-methylquinoline-3-carboxylate (100 g) in sulfolane (200 mL) was heated to 185° C. for 2 hours to give racemic ethyl 4-chloro-6-(2-cyanoethyl)-7-(2,3-dichlorophenyl)-8-fluoro-2-methylquinoline-3-carboxylate. The mixture was cooled to 50° C. and acetonitrile (200 mL) was added. To the solution was added water (700 mL) at 50° C. The mixture was cooled to room temperature and stirred for 4 hours. The solids were isolated by filtration and the wet cake was washed with water (2×200 mL). The wet solids were dried under vacuum at about 50° C. overnight to give desired product (97 g, 97% yield). LCMS calculated for C₂₂H₁₆Cl₃FN₂O₂: 464.03; Found: 465 (M+H⁺). ¹H-NMR (400 MHz, DMSO-d₆) δ 8.16 (s, 1H), 7.86 (dd, J=7.5, 2.1 Hz, 1H), 7.64-7.53 (m, 2H), 4.52 (q, J=7.1 Hz, 2H), 2.97-2.86 (m, 1H), 2.85-2.72 (m, 3H), 2.69 (s, 3H), 1.40 (t, J=7.1 Hz, 3H)

### Step 14. tert-Butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

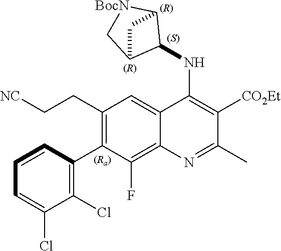

A mixture of ethyl (Rₐ)-4-chloro-6-(2-cyanoethyl)-7-(2,3-dichlorophenyl)-8-fluoro-2-methylquinoline-3-carboxylate (106.3 g, 228 mmol), tert-butyl (1R,4R,5S)-5-amino-2-azabicyclo[2.1.1]hexane-2-carboxylate (58.8 g, 297 mmol), lithium chloride (19 g, 446 mmol), diisopropylethylamine (99.5 g, 670 mmol) in dimethylsulfoxide (400 mL) was heated to 80° C. overnight. The reaction mixture was cooled to room temperature and tert-butyl methyl ether (TBME) (1 L) and water (500 mL) were subsequently added. The organic phase was separated. The organic phase was washed with 0.1N aqueous hydrochloric acid (500 mL), saturated sodium bicarbonate (500 mL) and water (500 mL). The solvent was removed under reduced pressure to give desired product that was used for next step without further purification. Analytical sample was purified by silica gel column (0-10% MeOH in DCM). LCMS calculated for C₃₂H₃₃Cl₂FN₄O₄: 626.19; Found: 627 (M+H⁺). ¹H-NMR (400 MHz, DMSO-d₆) δ 8.09 (s, 1H), 7.82 (dd, J=8.1, 1.5 Hz, 1H), 7.56 (t, J=7.8 Hz, 1H), 7.38 (dd, J=7.7, 1.5 Hz, 1H), 7.14 (s, 1H), 4.49-4.37 (m, 2H), 4.31 (s, 1H), 3.71 (d, J=4.1 Hz, 1H), 3.65-3.43 (m, 1H), 3.18 (d, J=9.3 Hz, 1H), 3.02 (s, 1H), 2.91-2.74 (m, 2H), 2.70 (dd, J=13.6, 5.9 Hz, 2H), 2.55 (s, 3H), 1.81-1.60 (m, 1H), 1.38 (t, J=7.1 Hz, 3H), 1.34-1.06 (m, 4H), 0.92 (s, 9H).

### Step 14a. tert-Butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate

The title compound can be alternatively prepared by the following method. A mixture of ethyl (R)-4-chloro-6-(2-cyanoethyl)-7-(2,3-dichlorophenyl)-8-fluoro-2-methylquinoline-3-carboxylate (40 g, 85 mmol), lithium carbonate (19 g, 258 mmol), and tert-Butyl (1R,4R,5S)-5-amino-2-azabicyclo[2.1.1]hexane-2-carboxylate oxalate (29.4 g, 98 mmol) in DMSO (120 mL) was heated to 80° C. overnight. The reaction mixture was cooled to r.t. and MTBE (300 mL) and filtered. The solids were rinsed with MTBE (100 mL). The combined filtrate was washed with water (2×320 mL). The organic phase was separated. The solvent was removed under reduced pressure to give the product that was used for next step without further purification. An analytical sample was purified by silica gel column (0-10% MeOH in DCM). LCMS calc. for C₃₂H₃₃Cl₂FN₄O₄: 626.19; Found: 627 (M+H⁺). ¹H-NMR (400 MHz, DMSO-d₆) δ 8.09 (s, 1H), 7.82 (dd, J=8.1, 1.5 Hz, 1H), 7.56 (t, J=7.8 Hz, 1H), 7.38 (dd, J=7.7, 1.5 Hz, 1H), 7.14 (s, 1H), 4.49-4.37 (m, 2H), 4.31 (s, 1H), 3.71 (d, J==4.1 Hz, 1H), 3.65-3.43 (m, 1H), 3.18 (d, J==9.3 Hz, 1H), 3.02 (s, 1H), 2.91-2.74 (m, 2H), 2.70 (dd, J=13.6, 5.9 Hz, 2H), 2.55 (s, 3H), 1.81-1.60 (m, 1H), 1.38 (t, J=7.1 Hz, 3H), 1.34-1.06 (m, 4H), 0.92 (s, 6H).

The alternative atropisomer tert-butyl (1R,4R,5S)-5-(((Rₐ)-6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate is prepared by an analogous route by performing an analogous process starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 15. (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

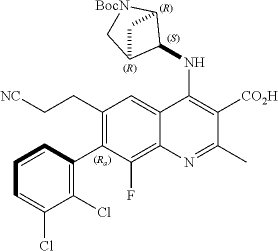

Sodium hydroxide aqueous solution (2 M) (134 mL, 268 mmol) was added to a solution of tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (140.0 g, 223 mmol) in acetonitrile (560 ml) and methanol (210 ml) at room temperature. The mixture was heated to 50° C. for 1-1.5 hours. The mixture was cooled to room temperature and acidified with 1M hydrochloric acid aqueous solution to about pH 5. The acetonitrile and methanol were removed under vacuum. The product was extracted by ethyl acetate (1.7 L). The aqueous phase was separated and extracted with ethyl acetate (420 mL). The combined ethyl acetate phases were concentrated under vacuum to give a residue. Tert-Butyl methyl ether (300 mL) was added to the residue and the mixture slurry was agitated at room temperature for 2 hours. The solids were isolated by filtration and the wet cake was washed with TBME (2×100 mL). The solids were dried under vacuum at about 50° C. to give desired product (135 g, quantitative) that was used for next step without further purification.

### Step 15b. (Rₐ)-4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid

The tile compound can be alternatively prepared by the following process. Sodium trimethylsinolate (338 g, 95%) was added to a solution of tert-butyl (1R,4R,5S)-5-((6-(2-cyanoethyl)-7-(2,3-dichlorophenyl)-3-(ethoxycarbonyl)-8-fluoro-2-methylquinolin-4-yl)amino)-2-azabicyclo[2.1.1]hexane-2-carboxylate (1400 g, 2.231 mol) in tetrahydrofuran (14 L) and water (80 mL) at room temperature. The mixture was heated to 50° C. for 1-3 hours to complete the reaction. The mixture was cooled to room temperature and acidified with 1M hydrochloric acid aqueous solution to about pH 5. The tetrahydrofuran was removed under vacuum. The product was extracted by dichloromethane (6 L). The aqueous phase was separated and extracted with dichloromethane (6 L). The combined organic phases were concentrated under vacuum to give the product in DCM solution (6 L). The concentrated dichloromethane solution was added to tert-butyl methyl ether (7 L) was added to the residue and the mixture slurry was agitated at room temperature for 2 hours. N-Heptane (7 L) was added to the mixture. The dichloromethane was removed under vacuum. The solids were isolated by filtration and the wet cake was washed with n-heptane (2×3 L). The solids were dried under vacuum at about 50° C. to give desired product that was used for next step without further purification.

### Step 16. tert-butyl (1R,4R,5S)-5-(((Rₐ)-6-(2-cyanoethyl)-7-(2,3-dichlorophenyl)-8-fluoro-3-iodo-2-methylquinolin-4-yl)amino)-2-azabicyclo[2.1.1]hexane-2-carboxylate

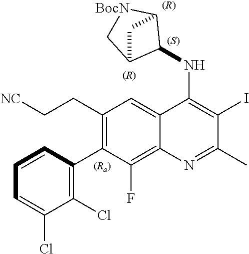

To a mixture of 4-(((1R,4R,5S)-2-(tert-butoxycarbonyl)-2-azabicyclo[2.1.1]hexan-5-yl)amino)-6-(2-cyanoethyl)-7-(2,3-dichlorophenyl)-8-fluoro-2-methylquinoline-3-carboxylic acid (132 g, 220 mmol), and sodium phosphate (74.4 g, 440 mmol) in anhydrous acetonitrile (1614 ml) was added N-iodosuccinimide (94 g, 396 mmol) and the mixture was stirred for 1 hour. Water (1.6 L) was added to the mixture and resulting slurry was stirred for 5 hours at room temperature. The solids were isolated by filtration and the wet cake was reslurried in water (2.6 L) at room temperature for 5 hours. The solids were isolated by filtration and the wet cake was washed with water (2×250 mL). The solids were dried under vacuum at about 50° C. to give desired product (120 g, 80% yield). LCMS calculated for C₃₉H₂₈Cl₂FIN₄O₂: 680.06; Found: 681 (M+H⁺). ¹H-NMR (400 MHz, DMSO-d₆) δ 7.94 (s, 1H), 7.82 (dd, J=8.0, 1.6 Hz, 1H), 7.56 (t, J=7.8 Hz, 1H), 7.50 (dd, J=7.7, 1.6 Hz, 1H), 5.49 (s, 1H), 4.28 (s, 2H), 3.09 (s, 1H), 2.96-2.58 (m, 8H), 1.71 (s, 1H), 1.59-0.96 (m, 11H).

### Step 17. tert-Butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl) ethynyl)-7-(2,3-dichlorophenyl)-8-fluoro-2-methylquinolin-4-yl)amino)-2-azabicyclo[2.1.1]hexane-2-carboxylate

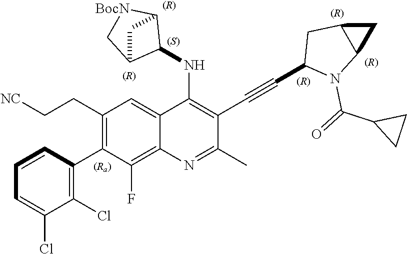

A mixture of cyclopropyl((1R,5R)-3-ethynyl-2-azabicyclo[3.1.0]hexan-2-yl) methanone (47.5 g, 260 mmol), tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate (136.5 g, 200 mmol), and tetrabutylammonium acetate (242 g, 801 mmol) in DMF (1100 ml) was subsurface purged with nitrogen gas for 10 minutes. Tris(dibenzylideneacetone)dipalladium(0) (2.75 g, 3 mmol) was added to the mixture. The mixture was subsurface purged with nitrogen gas for another 15 minutes before heating to 70° C. for 1 hour. The reaction mixture was cooled to room temperature and added to half saturated sodium bicarbonate aqueous solution (2200 mL). The solids were isolated by filtration and the wet cake was washed with water (600 mL). The solids were dried under vacuum at about 50° C. and purified by silica gel column eluted with 0-2% methanol in ethyl acetate to give desired product (142 g, 96% yield). ¹H NMR (400 MHz, DMSO-d₆) δ 8.03 (d, J=12.4 Hz, 1H), 7.81 (dd, J=8.1, 1.6 Hz, 1H), 7.55 (t, J=7.9 Hz, 1H), 7.36 (d, J=7.3 Hz, 1H), 6.70-6.44 (m, 1H), 5.68-5.13 (m, 1H), 4.54-4.18 (m, 2H), 4.00-3.80 (m, 1H), 3.51 (s, 1H), 3.19 (t, J=9.0 Hz, 1H), 3.07-2.91 (m, 1H), 2.78 (d, J=10.7 Hz, 3H), 2.66 (d, J=9.0 Hz, 3H), 2.57 (d, J=11.7 Hz, 4H), 2.36-2.08 (m, 2H), 1.88 (dd, J=17.9, 10.5 Hz, 2H), 1.35 (d, J=9.7 Hz, 2H), 1.15-0.59 (m, 16H).

### Step 17a. tert-Butyl (1R,4R,5S)-5-(((Rₐ)-6-(2-cyanoethyl)-3-(((1R,3R,5R)-2-(cyclopropanecarbonyl)-2-azabicyclo[3.1.0]hexan-3-yl) ethynyl)-7-(2,3-dichlorophenyl)-8-fluoro-2-methylquinolin-4-yl)amino)-2-azabicyclo[2.1.1]hexane-2-carboxylate

The title compound can alternatively be prepared by the following method. A mixture of cyclopropyl((1R,5R)-3-ethynyl-2-azabicyclo[3.1.0]hexan-2-yl) methanone (17.7 kg, 101 mol), tert-butyl (1R,4R,5S)-5-((6-(2-cyanoethyl)-7-(2,3-dichlorophenyl)-8-fluoro-3-iodo-2-methylquinolin-4-yl)amino)-2-azabicyclo[2.1.1]hexane-2-carboxylate (64.7 kg, 95 mol), copper (I) iodide (0.42 kg 2 mol), tris (4-fluorophenyl)phosphine (0.39 kg, 1 mol) and K₂CO₃ (36.4 kg, 191 mol) in DMSO (488.4 L) was subsurface purged with nitrogen gas for 30 min. Palladium (II) acetate (60 g, 30 mmol) was added to the mixture. The mixture was subsurface purged with nitrogen gas for another 30 min. before heating to 50° C. for more than 10 h. The reaction mixture was cooled to r.t. and EtOAc (906 L) was added, followed by slow addition of water (1267 L) was added. The mixture was stirred at r.t. for 30 min. and filtered over a diatomaceous earth bed. The diatomaceous earth bed was rinsed with EtOAc (33 L). The organic phase was separated from the aqueous phase and the aqueous phase was back extracted with EtOAc (195 L). The combined organic phase was washed with water (195 L). To the EtOAc phase was added water (130 L) and ammonium pyrrolidinedithiocarbamate (3.1 kg, 19 mol). The mixture was agitated at 50° C. for no less than 4 h. The mixture was cooled to r.t. and polish filtered. The aqueous phase was separated and discarded. The organic phase was washed with water (325 L). The organic phase was heated to 50° C. and passed through activated carbon cartridge. The solution is concentrated under vacuum and solvent swapped into toluene to remove residual water to give desired product in 98% solution yield. The toluene solution was solvent swap into NMP for next step indole-cyclization without further purification. ¹H NMR (400 MHz, DMSO-d₆) δ 8.03 (d, J=12.4 Hz, 1H), 7.81 (dd, J=8.1, 1.6 Hz, 1H), 7.55 (t, J=7.9 Hz, 1H), 7.36 (d, J=7.3 Hz, 1H), 6.70-6.44 (m, 1H), 5.68-5.13 (m, 1H), 4.54-4.18 (m, 2H), 4.00-3.80 (m, 1H), 3.51 (s, 1H), 3.19 (t, J=9.0 Hz, 1H), 3.07-2.91 (m, 1H), 2.78 (d, J=10.7 Hz, 3H), 2.66 (d, J=9.0 Hz, 3H), 2.57 (d, J=11.7 Hz, 4H), 2.36-2.08 (m, 2H), 1.88 (dd, J=17.9, 10.5 Hz, 2H), 1.35 (d, J=9.7 Hz, 2H), 1.15-0.59 (m, 16H).

The alternative atropisomer tert-butyl (1R,4R,5S)-5-(((Sₐ)-6-(2-cyanoethyl)-3-(((1R,3R,5R)-2-(cyclopropanecarbonyl)-2-azabicyclo[3.1.0]hexan-3-yl) ethynyl)-7-(2,3-dichlorophenyl)-8-fluoro-2-methylquinolin-4-yl)amino)-2-azabicyclo[2.1.1]hexane-2-carboxylate is prepared by an analogous route by performing processes analogous to Steps 14-17 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Step 18. tert-Butyl (1R,4R,5S)-5-((Rₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate

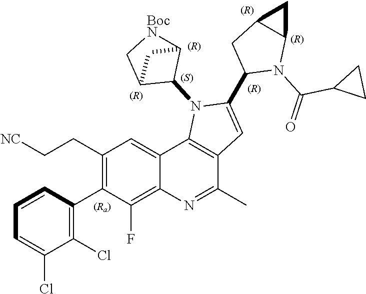

To a mixture of tert-butyl (1R,4R,5S)-5-((6-(2-cyanoethyl)-3-(((1R,3R,5R)-2-(cyclopropanecarbonyl)-2-azabicyclo[3.1.0]hexan-3-yl) ethynyl)-7-(2,3-dichlorophenyl)-8-fluoro-2-methylquinolin-4-yl)amino)-2-azabicyclo[2.1.1]hexane-2-carboxylate (141.0 g, 159 mmol) and cesium carbonate (78 g, 238 mmol) in dimethyl sulfoxide (1 L) or N-methy-2-pyrrolidone was heated to 80-85° C. for 1 hour and half. The reaction was cooled to room temperature and water (2 L) was gradually added. The product was gradually precipitated out of the solution. The resulting slurry was stirred at room temperature for 1 h. The solids were isolated by filtration and the wet cake was washed with water (2×300 mL). The wet solids were dried under vacuum. The solids were purified by flash chromatography with 60-100% ethyl acetate in dichloromethane. The solvents were removed and the solids in heptane (840 mL) were crystallized from ethyl acetate (420 mL) and tert-butyl methyl ether (420 mL) and heptane 9840 mL) to give desired product (122 g, 87% yield). LCMS calculated for C₄₀H₄₀Cl₂FN₅O₃: 727.25; Found: 728 (M+H⁺). ¹H NMR (500 MHz, DMSO-d₆) δ 8.12 (s, 1H), 7.81 (dt, J=8.0, 2.1 Hz, 1H), 7.55 (td, J=7.8, 5.0 Hz, 1H), 7.45-7.29 (m, 1H), 6.26 (s, 1H), 5.81-5.49 (m, 1H), 5.34-5.13 (m, 1H), 5.00 (dd, J=14.3, 6.8 Hz, 1H), 4.19-3.97 (m, 1H), 3.63 (dt, J=6.8, 3.1 Hz, 1H), 3.40 (d, J=9.4 Hz, 1H), 3.27-3.09 (m, 1H), 2.95 (dt, J=14.2, 7.6 Hz, 1H), 2.89-2.73 (m, 3H), 2.70 (d, J=2.7 Hz, 4H), 2.34-2.20 (m, 1H), 2.21-1.97 (m, 2H), 1.73 (dp, J=15.0, 4.8 Hz, 1H), 1.66-1.34 (m, 2H), 1.21-1.03 (m, 1H), 1.02-0.79 (m, 4H), 0.78-0.22 (m, 11H).

### Step 19. 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile

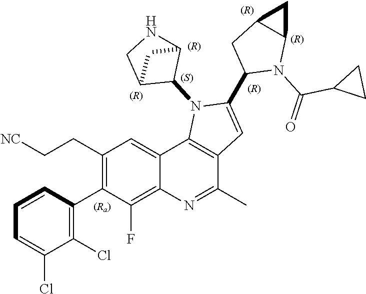

To a solution of tert-Butyl (1R,4R,5S)-5-((Rₐ)-8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate (167.7 g, 230.1 mmol) in dichloromethane (1.35 L) was added trimethylsilyl iodide (69 g, 345 mmol) at room temperature and stirred for 1 hour. Sodium bicarbonate aqueous solution (500 mL) was added to quench the reaction. The organic phase was isolated and washed with water. The solvent was evaporated by rotavapor and the residue was passed over silica gel bed (1-20% methanol in dichloromethane). The solvent was swapped into ethyl acetate and tert-butyl methyl ether to give crystalline product (136 g, 94% yield). LCMS calculated for C₃₅H₃₂Cl₂FN₅O: 627.20; Found: 628 (M+H⁺). ¹H-NMR (400 MHz, DMSO-d₆) δ 1H NMR (500 MHz, DMSO-d₆) δ 8.15 (d, J=13.6 Hz, 1H), 7.89-7.73 (m, 1H), 7.64-7.33 (m, 2H), 6.69-6.14 (m, 1H), 5.76-5.43 (m, 1H), 4.97 (d, J=4.9 Hz, 1H), 4.31 (dd, J=17.0, 6.0 Hz, 1H), 4.18-3.94 (m, 1H), 3.58-3.45 (m, 1H), 2.94 (dt, 2H, J=12.4, 6.1 Hz), 2.89-2.56 (m, 8H), 2.44-2.19 (m, 2H), 2.07 (d, J=12.9 Hz, 1H), 1.96-1.54 (m, 3H), 1.30-1.13 (m, 1H), 1.06-0.20 (m, 6H).

The alternative atropisomer 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile is prepared by an analogous route by performing processes analogous to Steps 14-19 starting from ethyl (Sₐ)-4-chloro-6-(2-cyanoethyl)-7-(2,3-dichlorophenyl)-8-fluoro-2-methylquinoline-3-carboxylate instead of ethyl (Rₐ)-4-chloro-6-(2-cyanoethyl)-7-(2,3-dichlorophenyl)-8-fluoro-2-methylquinoline-3-carboxylate.

### Example 2. 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile monohydrochloride dihydrate

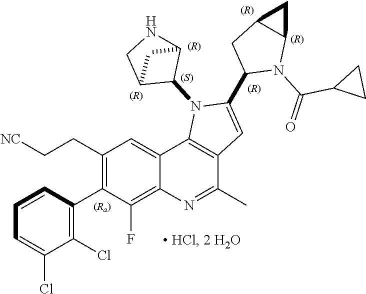

To a solution of dissolved 3-((Rₐ)-1-((1R,4R,5S)-2-azabicyclo[2.1.1]-hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile free base (53.8 g, 85 mmol) in methanol (110 mL), ethyl acetate (50 mL), water (11 mL) and tert-butyl methyl ether (TBME) (110 mL) was added 6N aqueous hydrochloric acid (14.5 mL) at 30-50° C. The mixture was seeded and the solution gradually turned cloudy. TBME (440 mL) was slowly added to the mixture at about 40° C. over 1 hour. The mixture was cooled to about 15° C. and agitated for 2 hours. The solids were isolated by filtration and the wet cake was washed with 5% methanol and 20% ethyl acetate in TBME (2×110 mL). The wet solids were slurried in ethyl acetate (270 mL) and dried under vacuum at about 50° C. to give desired product (53.7 g, 90% yield). LCMS calculated for C₃₅H₃₂Cl₂FN₅O: 627.20; Found: 628 (M+H⁺). ¹H NMR (500 MHz, DMSO-d₆) δ 8.15 (s, 1H), 7.83 (dd, J=8.1, 1.6 Hz, 1H); 7.57 (dd, J=7.9, 7.9, 1H); 7.45 (dd, J=7.7, 1.6 Hz, 1H); 6.44 (s, 1H); 5.65 (s, 1H); 5.51 (d, J=10.6 Hz, 1H); 4.14 (td, J=6.4, 2.6 Hz, 1H); 3.84-3.90 (m, 1H); 3.30-3.37 (m, 1H); 3.43-3.50 (m, 1H); 2.86-2.95 (m, 1H); 2.83-2.92 (m, 1H); 2.79 (s, 3H); 2.70-2.79 (m, 1H); 2.29-2.35 (m, 1H); 2.25-2.32 (m, 1H); 1.97 (dd, J=13.0, 2.6 Hz, 1H); 1.69-1.83 (m, 1H); 1.65 (d, J=9.1 Hz, 1H); 0.91-1.00 (m, 2H); 0.82-0.88 (m, 2H); 0.72-0.80 (m, 1H); 0.63-0.69 (m, 1H). 13C NMR (125 MHz, DMSO-d₆) δ 171.6; 145.8; 132.8; 135.1; 132.8; 131.9; 131.5; 131.4; 129.2; 101.6; 120.7; 57.9; 56.5; 44.5; 42.5; 30.5; 38.3; 32.8; 22.1; 17.5; 17.1; 13.2; 13.0; 7.70; 7.80. 19F NMR (376 MHz, DMSO-d₆) δ −122.1 (s).

The alternative atropisomer 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile monohydrochloride dihydrate is prepared by an analogous route by performing processes analogous to Steps 15-21 starting from 3-((Sₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile instead of 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

### Example 3. Synthesis of cyclopropyl((1R,3R,5R)-3-ethynyl-2-azabicyclo[3.1.0]hexan-2-yl)methanone (Step 17a in Example 1)

### Step 1. 1-(tert-Butyl) 2-ethyl (R)-2,3-dihydro-1H-pyrrole-1,2-dicarboxylate

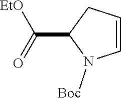

To a solution of 1-(tert-butyl) 2-ethyl (R)-5-oxopyrrolidine-1,2-dicarboxylate (241 g, 0.938 mol) in anhydrous toluene (1.6 L) was added 1M lithium triethyl borohydride in tetrahydrofuran (1.01 L, 1.01 mol) dropwise at −50-−40° C. over 1 h. After addition, the mixture was stirred for 1 h at about −50° C. DIPEA (726 mL, 4.17 mol) was added to the mixture dropwise over 1 h. 4-Dimethylaminopyridine (1.49 g, 12.2 mmol, 0.013 eq.) was added to the mixture, followed by the dropwise addition of trifluoroacetic anhydride (156.5 mL, 1.126 mol) over 1.5 h. After addition, the mixture was stirred for 1 h at about −50° C., then slowly warmed to r.t. The mixture was stirred for 1 h at r.t. The reaction mixture was cooled to 0° C. and diluted slowly with water (2.41 L), while maintaining the temperature below 10° C. during addition. The organic layer was separated and washed with water (2.41 L) and saturated brine (720 mL). The organic layer was dried over sodium sulfate (120 g). The solution was concentrated under reduced pressure to give desired product (230 g, quant.) as yellow oil. GCMS calc. for C₁₂H₁₉NO₄: 241.1; Found: 214.2 (M+). ¹H-NMR (400 MHz, CDCl₃) δ 6.70-6.48 (m, 1H), 4.99-4.86 (m, 1H), 4.70-4.52 (m, 1H), 4.30-4.11 (m, 2H), 3.15-2.98 (m, 1H), 2.73-2.57 (m, 1H), 1.53-1.38 (m, 9H), 1.34-1.21 (m, 4H).

### Step 2. 2-(tert-Butyl) 3-ethyl (1R,3R,5R)-2-azabicyclo[3.1.0]hexane-2,3-dicarboxylate

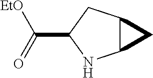

To a solution 1-(tert-butyl) 2-ethyl (R)-2,3-dihydro-1H-pyrrole-1,2-dicarboxylate (230 g, 0.938 mol) in toluene (2.3 L) was added 1.1M diethylzinc in toluene (1.7 L, 1.87 mol) at −30 to −25° C. over 1 h. Chloroiodomethane (273 mL, 3.752 mol) was added to the mixture dropwise over 2 h at about −30 to −20° C. and the mixture was stirred for 16 h. Half-saturated sodium bicarbonate (2.3 L) was added to the mixture and the mixture was warmed up to r.t. The mixture was filtered over diatomaceous earth to remove white solids and the filter bed was rinsed with toluene (1.5 L). The organic layer was separated from the filtrate and washed with water (2×1.15 L) and saturated brine (1.15 L). The toluene solution was concentrated under reduced pressure to give a 6 to 1 mixture (231 g) of 2-(tert-Butyl) 3-ethyl (1R,3R,5R)-2-azabicyclo[3.1.0]hexane-2,3-dicarboxylate and 2-(tert-butyl) 3-ethyl (1S,3R,5S)-2-azabicyclo[3.1.0]hexane-2,3-dicarboxylate as yellow oil as determined by GCMS analysis.

Aqueous methyl amine (40%, 344 g) was added to a crude mixture product obtained above (226 g) and the mixture was stirred for 16 h at r.t. Water (340 mL) and methyl tert-butyl ether (340 mL) was added to the mixture. The organic layer was separated and washed with water (340 mL) and saturated brine (230 mL). The solution was concentrated under reduced pressure to give 2-(tert-butyl) 3-ethyl (1R,3R,5R)-2-azabicyclo[3.1.0]hexane-2,3-dicarboxylate (177 g, 73% calc. yield) as yellow oil, which contained 2% 2-(tert-butyl) 3-ethyl (1S,3R,5S)-2-azabicyclo[3.1.0]hexane-2,3-dicarboxylate as determined by GCMS analysis. GCMS calc. for C₁₃H₂₁NO₄: 255.1; Found: 255.1 (M⁺). ¹H-NMR (400 MHz, CDCl₃) δ 4.56-4.39 (m, 1H), 4.18-4.01 (m, 2H), 3.51-3.36 (m, 1H), 2.60-2.42 (m, 1H), 2.00-1.92 (m, 1H), 1.45-1.32 (m, 9H), 1.23-1.15 (m, 4H), 0.87-0.79 (m, 1H), 0.70-0.56 (m, 1H).

### Step 3. tert-Butyl (1R,3R,5R)-3-(hydroxymethyl)-2-azabicyclo[3.1.0]hexane-2-carboxylate

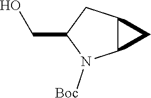

A solution of 2-(tert-butyl) 3-ethyl (1R,3R,5R)-2-azabicyclo[3.1.0]hexane-2,3-dicarboxylate (177 g, 0.694 mol) in tetrahydrofuran (1.56 L) was added to 1M lithium aluminum hydride solution in tetrahydrofuran (777 mL, 0.777 mol, 1.12 eq.) at about 0-10° C. over 1 h. After addition, the mixture was stirred for 2 h at 3° C. Water (27 mL) was added to the mixture dropwise to quench the reaction. Sodium hydroxide solution (15%, 27 mL) and water (80 mL) were sequentially added to the mixture dropwise. The mixture was stirred at r.t. for 1 h. DCM (2.35 L) was added to the mixture. The suspension was filtered through diatomaceous earth (100 g) bed and rinsed with DCM (300 mL). The filtrate was concentrated under reduced pressure and dried under vacuum oven at 40° C. for 18 h to give tert-butyl (1R,3R,5R)-3-(hydroxymethyl)-2-azabicyclo[3.1.0]hexane-2-carboxylate (133 g, 90% yield) as yellow oil which contained 2% of an isomer as determined by GCMS analysis. GCMS calc. for C₁₁H₁₉NO₃: 213.1; Found: 213.2 (M⁺). ¹H-NMR (400 MHz, CDCl₃) δ 4.83 (brs, 1H), 4.34 (brs, 1H), 2.45 (ddd, 1H), 1.55-1.43 (m, 12H), 0.80 (q, 1H), 0.40 (brs, 1H).

### Step 4. tert-Butyl (1R,3R,5R)-3-formyl-2-azabicyclo[3.1.0]hexane-2-carboxylate

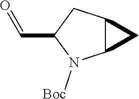

DMSO (42.7 mL, 0.603 mol) was added to oxalyl chloride (26.4 mL, 0.301 mol) in DCM (535 mL) dropwise at −78° C. over 30 min., while maintaining the temperature below −60° C. during addition. After stirring at −78° C. for 30 min. tert-butyl (1R,3R,5R)-3-(hydroxymethyl)-2-azabicyclo[3.1.0]hexane-2-carboxylate (53.5 g, 0.251 mol) in DCM (535 mL) was added to solution dropwise at −78° C. over 40 min. After stirring at −78° C. for 30 min., NEt₃ (104.9 mL, 0.753 mol) was added to solution dropwise at −78° C. over 40 min. After stirring at −78° C. for 1 h, the reaction mixture was warmed to 0° C. and stirred for 30 min. Water (888 mL) was added to the mixture and stirred for 20 min. The aqueous layer was separated and extracted with DCM (2×888 mL). The combined organic layers were sequentially washed with 1 M HCl (888 mL), water (888 mL) and saturated brine (888 mL). The organic layer was concentrated under reduced pressure to give tert-butyl (1R,3R,5R)-3-formyl-2-azabicyclo[3.1.0]hexane-2-carboxylate (44 g, 83% yield) as yellow oil. GCMS calc. for C₁₁H₁₇NO₃: 213.1; Found: 213.2 (M⁺). ¹H-NMR (400 MHz, CDCl₃) δ 9.54-9.31 (m, 1H), 4.64-4.39 (m, 1H), 3.68-3.45 (m, 1H), 2.68-2.33 (m, 1H), 2.24-2.10 (m, 1H), 1.53-1.41 (m, 10H), 0.88-0.71 (m, 1H), 0.39-0.28 (m, 1H).

### Step 5. tert-Butyl (1R,3R,5R)-3-ethynyl-2-azabicyclo[3.1.0]hexane-2-carboxylate

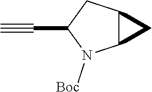

K₂CO₃ (28.8 g, 0.209 mol, 2 eq.) was added to a solution of tert-butyl (1R,3R,5R)-3-formyl-2-azabicyclo[3.1.0]hexane-2-carboxylate (22 g, 0.104 mol) in methanol (352 mL) at 0-5° C. Dimethyl (1-diazo-2-oxopropyl)phosphonate (18.3 mL, 0.110 mol) was added to the mixture dropwise at 0-5° C. for 30 min., while maintaining the temperature at <5° C. during addition. After stirring at 0-5° C. for 15 min., the reaction mixture was warmed up to r.t. and stirred for 2 h. Water (372 mL) and EtOAc (930 mL) was added to the mixture, which was stirred for 15 min. The aqueous layer was separated and extracted with EtOAc (372 mL). The combined organic layers were washed with water (560 mL) and saturated brine (560 mL). The organic solution was concentrated under reduced pressure and purified over silica gel and eluted with a gradient of 0-10% EtOAc in heptane to give a 7 to 1 mixture of tert-butyl (1R,3R,5R)-3-ethynyl-2-azabicyclo[3.1.0]hexane-2-carboxylate and tert-butyl (1R,3S,5R)-3-ethynyl-2-azabicyclo[3.1.0]hexane-2-carboxylate (82 g, 74% calc. yield) as light yellow oil. GCMS calc. for C₁₂H₁₇NO₂: 207.1; Found: 207.0 (M+). ¹H-NMR (400 MHz, CDCl₃) δ 4.78-4.54 (m, 1H), 3.60-3.46 (m, 1H), 2.52-2.40 (m, 1H), 2.30-2.22 (m, 1H), 2.18-2.08 (m, 1H), 1.50-1.48 (m, 9H), 1.16-1.05 (m, 1H), 0.91-0.80 (m, 1H), 0.78-0.66 (m, 1H).

### Step 6. Cyclopropyl((1R,3R,5R)-3-ethynyl-2-azabicyclo[3.1.0]hexan-2-yl) methanone

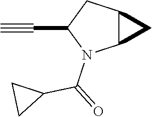

A mixture of tert-butyl (1R,3R,5R)-3-ethynyl-2-azabicyclo[3.1.0]hexane-2-carboxylate and tert-butyl (1R,3S,5R)-3-ethynyl-2-azabicyclo[3.1.0]hexane-2-carboxylate (82 g, 0.39 mol) and 4M HCl in dioxane (297 mL, 1.19 mol, 3 eq.) was stirred at rt for 4 h. The reaction mixture was diluted with THF (1.23 L) and cooled to 0° C. NEt₃ (275.8 mL, 1.98 mol) was added to the reaction at 0° C. dropwise over 1.5 h while maintaining the temperature at <10° C. during addition. Cyclopropanecarbonyl chloride (45.4 g, 0.43 mol) was added to the reaction at 0° C. The reaction was warmed to r.t. and stirred for 3 h. 1M HCl (410 mL, 5 vol) and DCM (820 mL) was added. The aqueous layer was separated and extracted with DCM (2×820 mL). The combined organic layers were washed with water (820 mL) and saturated brine (820 mL). The organic layer was concentrated under reduced pressure to give a crude residue (60 g). Diatomaceous earth (120 g) was added to the crude residue and the mixture was dried under reduced pressure to give a dried load powder (186 g). The dried load powder was purified on a silica gel column (1.5 kg) and eluted with a gradient of 15 to 40% EtOAc in heptane. The desired fractions were concentrated under reduced pressure and dried under vacuum at 30° C. for 18 h to give the title compound (40.8 g, 59% yield) as brown oil. GCMS calc. for C₁₂H₁₇NO₂: 175.1; Found: 175.0 (M+). ¹H-NMR (400 MHz, DMSOd₆) δ 5.14 (dt, 0.45H), 4.81 (dt, 0.55H), 3.82 (t, 0.55H), 3.71 (t, 0.45H), 3.42 (d, 0.45H), 3.15 (d, 0.55H), 2.57 (ddd, 0.45H), 2.44 (ddd, 0.55H), 2.09 (dd, 0.45H), 2.04 (ddd, 0.55H), 1.97 (dd, 0.55H), 1.86-1.69 (m, 1H), 1.62 (dddd, 0.45H), 1.01 (td, 0.55H), 0.90 (td, 0.45H), 0.87-0.68 (m, 5H).

### Example 4. Synthesis of tert-Butyl (1R,4R,5S)-5-amino-2-azabicyclo[2.1.1]hexane-2-carboxylate oxalate (Step 14a in Example 1)

### Step 1. (E)-4-Methoxybut-3-en-2-one

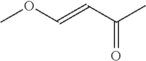

A mixture of 4,4-dimethoxy-2-butanone (350 g, 1.0 eq) and sodium acetate (11 g, 0.05 eq.) was heated to 145-150° C. under nitrogen atmosphere and the resulting methanol is purged during the heating process. When the reaction is complete, the mixture was cooled to 70-80° C. The product was distilled under vacuum to give desired product (130 g, yield 50%). ¹H NMR (CD₂Cl₂/CHDOD, 400 MHz): δ 7.60 (d, 1H, J=12.8 Hz) 5.53 (d, 1H, J=12.8 Hz), 3.81 (s, 3H), 2.17 (s, 3H). 13C NMR (CD₂Cl₂/CD₃OD, 100.6 MHz): δ 27.1, 58.0, 107.0, 165.2, 199.6.

### Step 2. (E)-4-(Allylamino) but-3-en-2-one

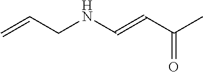

A mixture of (E)-4-methoxybut-3-en-2-one (150 g) and NEt₃ (182 g) in DCM (450 mL) was added was agitated under nitrogen at 10-15° C. Allylamine hydrochloride aqueous solution (60%, 234 g) is slowly added to the mixture at 10-15° C. After the addition, the mixture is agitated for 30 min. When the reaction was completed, water (150 g) was added to the reaction mixture. The organic phase was separated, and the water phase was extracted with DCM (300 mL). The combined organic phases were washed with brine (150 mL) and organic phase was concentrated under vacuum to give crude product as yellow oil (175 g, yield 93%). ¹H NMR (500 MHz, CDCl₃): δ 9.75 (bs, 1H); 6.58 (dd, 1H, J=16.8, 2); 5.78-5.86 (m, 1H); 5.19 (d, 1H, J=16.8)); 5.14 (d, 1H, J=10, 1)); 5.00 (d, 1H, J=10, 1); 3.74-3.77 (m, 2H); 2.03, (s, 3H). 13C NMR (125 Hz, CDCl₃): 197.5; 153.2; 165.3; 117.6; 94.9; 51.1; 29.2.

### Step 3. tert-Butyl (E)-allyl(3-oxobut-1-en-1-yl) carbamate

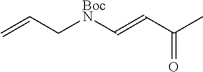 [SMILES: C=CCN(/C=C/C(C)=O)C(=O)OC(C)(C)C]

A mixture of (E)-4-(allylamino) but-3-en-2-one (130 g), trimethylamine (105 g), N,N-dimethylaminopyridine (13 g) in toluene (390 mL) was heated to 50-55° C. (Boc) 20 (259 g) was added in portion while maintained the reaction temperature between 50-55° C. After the reaction mixture was agitated for 2 h at 50-55° C. to complete the reaction. The mixture was cooled to 10-15° C. and 3 M aq. HCl was added to the mixture until the pH 5-6. The organic phase was separated, and the aqueous phase was extracted with toluene (260 mL). The combined organic phases were washed with water (260 mL). Activated charcoal (1 g) was added. The mixture was agitated at 50-55° C. for 1 h before cooling the mixture to 20-30° C. The mixture was filtered over diatomaceous earth bed and the diatomaceous earth bed was rinsed with toluene. The filtrated was concentrated to a residue and the residue was coevaporated with MeCN to give a residue as yellow oil (189 g, 80% yield). ¹H NMR (500 MHz, CDCl₃): δ 8.11 (d, 1H, J=15); 5.68-5.73 (m, 1H); 5.49 (d, 1H, J=15); 5.14 (d, 1H, J=18); 5.09 (d, 1H, J=10); 4.13 (t, 2H); 2.20 (s, 3H); 1.50 (s, 9H). 13C NMR (125 MHz, CDCl₃): 198.6; 153.0; 143.2; 131.8; 117.8; 109.5; 84.0; 47.0; 28.3; 28.1.

### Step 4. tert-Butyl 5-acetyl-2-azabicyclo[2.1.1]hexane-2-carboxylate

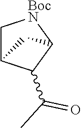

A solution of tert-butyl (E)-allyl(3-oxobut-1-en-1-yl) carbamate (270 g) in MeCN (3240 mL) was subjected to UV-photo reactor. When the reaction was complete, the yellow oil residue (major and minor isomer mixture) was used for next step without further purification. Sample was purified by column to get analytical data. ¹H NMR (500 MHz, CDCl₃) δ 4.62-6.78 (bd, 1H); 3.40 (bt, 1H); 3.16 (bs, 1H); 3.06 (bs, 1H); 2.69 (s, 1H); 1.97 (s, 3H); 1.70-1.73 (m, 1H); 1.46 (s, 9H).

### Step 5. tert-Butyl (1R,4R,5S)-5-amino-2-azabicyclo[2.1.1]hexane-2-carboxylate oxalate

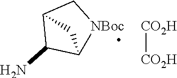 [SMILES: CC(C)(C)OC(=O)N1C[C@H]2C[C@@H]1[C@H]2N ; O=C(O)C(=O)O]

A mixture of tert-butyl 5-acetyl-2-azabicyclo[2.1.1]hexane-2-carboxylate (150 g) in MeCN (1500 mL) was added to sodium hypochlorite (173.5 g) in 30% sodium hydroxide solution at 30-40° C. (1500 mL). The mixture was agitated at 30-40° C. for 30 min. to complete the reaction. The mixture was cooled to 10-15° C. and 6M HCl aq. solution was added to adjust the mixture pH 8-9. The mixture was concentrated under vacuum to remove MeCN at 50-55° C. and methanol (90 mL) was added to the residue. The mixture was cooled to 10-15° C. and 6M HCl was added to adjust the mixture pH 2-3 (solids precipitated out as the pH adjustment) and agitated for additional 2-3 h. The solids were isolated and rinsed with water (300 mL). The wet solids were dried under vacuum at 50-55° C.

Recrystallization: A mixture of the solids in toluene (1500 mL) was heated to 60-70° C. to a solution. (R)-(+)-1-phenylethylamine (80.7 g) was added at 40-70° C. The solution was cooled to 30-35° C. over 90 min. (solids precipitated gradually) and agitated for 1 h. The suspension was cooled to 20-25° C. over 90 min. and agitated for 2 h. The solids were isolated and rinsed with toluene (40 mL). A mixture of the cake and toluene (1200 mL) was heated to 100-105° C. to a solution. The mixture was cooled to 75-85° C. over 90 min. (solids precipitated) and agitated for 1 h. The mixture was cooled to 20-25° C. over 2 h and agitated for 2 h. The solids were isolated and rinsed with toluene (40 mL). The recrystallization process was repeated one more time.

Free base: to a mixture of the wet cake in toluene (225 mL) and water (225 mL) was added 30% aq. NaOH at 10-15° C. to pH 9-10. The mixture was agitated for 30 min. and the organic phase was separated. To the aqueous phase was added 6 M aq. HCl at 10-15° C. to pH 2-3 (solids predicated). The mixture was then cooled to 3-8° C. and agitated for 1 h. The solids were isolated and washed with water (40 mL). The wet cake was dried under vacuum at 50-55° C. to give the desired (1R,4S,5S)-2-(tert-butoxycarbonyl)-2-azabicyclo[2.1.1]hexane-5-carboxylic acid (25 g, 18% yield).

A mixture of the acid (245 g), pyridine (86 g) and ammonium carbonate (111 g) in MeCN (3700 mL) was added (Boc)₂O (310 g) at 15-25° C. The mixture was agitated for 5 h to complete the reaction. The solids were isolated and rinsed with MeCN (250 mL). The filtrate and rinse were combined and concentrated under vacuum at 40-45° C. and azeotroped with heptane. To the residue was added EtOAc (130 mL) and n-heptane (650 mL) at 40-45° C. The mixture was cooled to 10-15° C. (solids precipitated) and agitated for 2 h. The solids were isolated and rinsed with n-heptane (250 mL). The wet cake was dried under vacuum at 50-55° C. to give desired product tert-butyl (1R,4S,5S)-5-carbamoyl-2-azabicyclo[2.1.1]hexane-2-carboxylate quantitatively.

To cooled 15% aq. NaOH (800 mL) at 10-15° C. was added the tert-butyl (1R,4S,5S)-5-carbamoyl-2-azabicyclo[2.1.1]hexane-2-carboxylate (214 g). Sodium hypochlorite (91.2 g) was added at 10-20° C. and the mixture was agitated for 2 h. The mixture was heated to 40-45° C. for 4 h to complete the reaction. The reaction mixture was cooled to 15-20° C. and citric acid was added to adjust pH 5-6. The mixture was basified by addition of sodium hydroxide to pH 14. The basified mixture was extracted with 2-methyltetrahydrofuran (2×1000 mL). The combined organic phase was concentrated under vacuum and the residual was azeotroped with MeCN. The residue was dissolved in (140 mL) and activated charcoal (2 gram) was added. The mixture was agitated at 25-30° C. for 2 h. The mixture was filtered, and the filter bed is rinsed with MeCN (85 mL). The combined filtrate and rinse were added to a solution of oxalic acid (120 g) in MeCN (850 mL) at 40-45° C. The solution was cooled to 3-7° C. and agitated for 1 h. The solids were isolated and rinsed with MeCN (110 mL). The wet cake was dried at 40-50° C. under vacuum to give desired tert-butyl (1R,4R,5S)-5-amino-2-azabicyclo[2.1.1]hexane-2-carboxylate oxalate (248 g, 91% yield) as white solids. HPLC-MS for calc. C₁₀H₁₈N₂O₂: 198.14; Found (M+H): 199.1 ¹H NMR (500 MHz, DMSO-d₆): δ 8.44 (s, 3H); 3.34, (m, 1H); 4.24, dt, 1H, J=6.9, 1.7 Hz); 3.20-3.31 (m, 2H); 2.84, (dt, 1H, J=6.5, 3.0); 1.65-1.71 (m, 1H); 1.42 (s, 9H); 1.19 (d, 1H, J=8.1). ¹³C NMR (125 Hz, DMSO-d₆): δ 165.0; 155.8; 79.5; 61.5; 50.6; 44.9; 40.8; 33.8; 28.6.

### Example 5. Characterization Parameters

### X-Ray Powder Diffraction (XRPD)

The X-Ray Powder Diffraction (XRPD) was obtained from Bruker D8 Advance ECO X-ray Powder Diffractometer (XRPD) instrument. The general experimental procedures for XRPD were: (1) X-ray radiation from copper at 1.5418 Å and LYNXEYE™ detector; (2) X-ray power at 40 kV, 25 mA; and (3) the sample powder was dispersed on a zero-background sample holder. The general measurement conditions for XRPD were: Start Angle 3 degrees; Stop Angle 30 degrees; Sampling 0.015 degrees; and Scan speed 2 degree/min.

Differential Scanning calorimetry (DSC)

The DSC was obtained from TA Instruments Differential Scanning calorimetry, Discovery DSC2500 with autosampler. The DSC instrument conditions were as follows: 20-300° C. at 10° C./min; Tzero aluminum sample pan and lid; and nitrogen gas flow at 50 mL/min.

### Thermogravimetric Analysis (TGA)

The TGA was obtained from TA Instruments Thermogravimetric Analyzer, Discovery TGA5500 with autosampler. The general experimental conditions for TGA were: ramp from 25° C. to 300° C. at 10° C./min; nitrogen purge gas flow at 25 mL/min; platinum sample holder.

### Example 6. Compound 1 Hydrochloride Polymorph Screening

### Solubility Measurement

The solubility of the Compound 1 HCl sample (prepared according to Example 4) was measured according to the procedure for solubility at 25° C. in Table 8 and the procedure for solubility at 50° C. in Table 9. The results are summarized in Table 10.

TABLE 8

| Op# | Operation |
|---|---|
| 1 | Added 5 mL solvents listed in the Table 11 to the individual vials |
| 2 | Added Compound 1 HCl to the vials to get a cloudy solution at 25° C. |
| 3 | Added another approximately 20 mg Compound 1 HCl to the cloudy solution. |
| 4 | Agitated the mixture at 25 ± 1° C. for 48 h, which is controlled by IKA ® ETS-D5 |
| | temperature controller and IKA ® RCT basic safety control |
| 5 | Filtered the supernatant using syringe filter (0.22 μm) |
| 6 | Pipetted the saturated solution into HPLC vials. |
| 7 | Diluted the saturated solution in HPLC vials with MeOH or acetone. |
| 8 | HPLC analysis and calculated the corresponding solubility as indicated in Table 11. |

TABLE 9

| Op# | Operation |
|---|---|
| 1 | Added 5 mL solvents listed in the Table 11 to the individual vials |
| 2 | Added Compound 1 HCl to cloudy solution at 50° C. |
| 3 | Added another ~20-25 mg of Compound 1 HCl. |
| 4 | Agitated the mixture at 50 ± 1° C. for 24 h, which is controlled by IKA ® ETS-D5 |
| | temperature controller and IKA ® RCT basic safety control. |
| 5 | Filtered quickly the supernatant using warmed syringe filter at 50 ± 1° C. (0.22 μm). |
| 6 | Pipetted the saturated solution into HPLC vials. |
| 7 | Diluted the saturated solution in HPLC vials with MeOH or acetone. |
| 8 | HPLC analysis and calculated the corresponding solubility as indicated in Table 11. |

TABLE 10

| | Solvent | Solubility (25° C.) | Solubility (50° C.) |
|---|---|---|---|
| 1 | MeCN | 2.3 | 3.7 |
| 2 | Chloroform | 7.8 | 21.2 |
| 3 | Dichloromethane | 2.4 | 4.7 |
| 4 | Dimethylformamide | >50* | >50* |
| 25 | 1,4-Dioxane | 3.7 | 8.2 |
| 6 | MeOH | >50* | >50* |
| 7 | 2-Methoxyethanol | >50* | >50* |
| 8 | Methyl iso-butyl | 1.3 | 2.7 |
| | ketone | | |
| 9 | Toluene | 0.05 | 0.1 |
| 10 | Acetone | 10.1 | 19.2 |
| 11 | n-BuOH | >50* | >50* |
| 12 | tert-Butyl methyl | 0.03 | 0.1 |
| | ether | | |
| 13 | DMSO | >50* | >50* |
| 14 | EtOH | 10.2 | 22.3 |
| 15 | EtOAc | 0.2 | 0.4 |
| 16 | Ethyl formate | 0.3 | 0.6 |
| 17 | Heptane | 0.01 | 0.02 |
| 18 | iso-Butyl acetate | 0.1 | 0.3 |
| 19 | iso-Propyl acetate | 0.1 | 0.3 |
| 20 | n-Propanol | 12.7 | >50* |
| 21 | iso-Propanol (IPA) | 11.2 | >50* |
| 22 | Water | NA^{#} | NA^{#} |
| 23 | Methyl ethyl | 3.2 | 5.7 |
| | ketone | | |
| 24 | Tetrahydrofuran | 0.9 | 2.2 |
| | (THF) | | |
| 25 | 2-Methyl THF | 0.2 | 0.4 |
| 26 | 66% MTBE 20% | 13.2 | 31.2 |
| | MeOH | | |
| | 10% EtOAc 4% | | |
| | water | | |
| 27 | 3% MeOH 0.3% | 0.4 | 0.8 |
| | water in EtOAc | | |

*by visual assessment.
^{#}The mixture was emulsified during the stirring.

At 25° C., Compound 1 HCl has excellent solubility in (>50 mg/mL) in MeOH, n-butanol, dimethylformamide (DMF), 2-methoxyethanol, and DMSO. It is slightly soluble (1 mg/mL<solubility<15 mg/mL) in acetonitrile, chloroform, dichloromethane, 1,4-dioxane, acetone, methyl ethyl acetone, methyl iso-butyl ketone, EtOH, n-propanol, iso-propanol, and MTBE/MeOH/EtOAc/water (volume ratio 66:20:10:4). It has poor solubility (<1 mg/mL) in toluene, tert-butyl methyl ether (MTBE), EtOAc, iso-propyl acetate, iso-butyl acetate, ethyl formate, tetrahydrofuran (THF), 2-methyl THF, and MeOH/water/EtOAc (volume ratio 3:0.3:96.7). It is completely insoluble in heptane.

At 50° C., Compound 1 HCl has excellent solubility in (>50 mg/mL) in MeOH, n-propanol, iso-propanol, n-butanol, dimethylformamide (DMF), 2-methoxyethanol, and DMSO. It has relatively good solubility (15 mg/mL<solubility<50 mg/mL) in chloroform, acetone, EtOH, and MTBE/MeOH/EtOAc/water (volume ratio 66:20:10:4). It is slightly soluble (1 mg/mL<solubility<15 mg/mL) in acetonitrile, dichloromethane, 1,4-dioxane, tetrahydrofuran (THF), methyl ethyl ketone, and methyl iso-butyl ketone. It has poor solubility (<1 mg/mL) in toluene, tert-butyl methyl ether (MTBE), ethyl formate, EtOAc, iso-propyl acetate, iso-butyl acetate, 2-methyl THF, and MeOH/water/EtOAc (volume ratio 3:0.3:96.7). It is completely insoluble in heptane.

### Phase Equilibration at 25±1° C. and 50±1° C.

Phase equilibration studies were designed to provide information on a predominant crystal form for phase identification. Based on its solubility in various solvent systems (Table 10), Compound 1 HCl was equilibrated in the representative groups of solvents at 25±1° C. (Table 11) and 50±1° C. (Table 12). To the solvents listed in Table 11 and Table 12, Compound 1 HCl was added until a cloudy solution was obtained, then, approximately 20 mg of Compound 1 HCl was added to the cloudy solution. The mixture was stirred at 25±1° C. and 50±1° C. for 48 hours and 24 hours, respectively. The solid was filtered, dried in vacuum, and analyzed by XRPD to give the results in Table 11 and Table 12. Five potential new forms (Forms II-VI) were obtained.

The Compound 1 HCl solids obtained after phase equilibration at 25° C. in n-BuOH, ethyl formate, n-propanol, methyl ethyl ketone (MEK), 2-methyltetrahydronfuran (2-methyl THF), and THF have a different XRPD patterns when comparing to that of the staring material. These new crystalline forms were named as Forms II-VI.

The Compound 1 HCl solids obtained after phase equilibration at 50° C. in ethyl formate, MEK, 2-methyl THF, and THF have Forms III, V, V, and VI, respectively.

TABLE 11

| Exp. No. | Solvent | Solid Form |
|---|---|---|
| Compound | N/A | I |
| 1 HCl | | |
| 1 | MeCN | I |
| 2 | Chloroform | I |
| 3 | DCM | I |
| 4 | DMF | |
| 5 | 1,4-Dioxane | I |
| 6 | MeOH | |
| 7 | 2-Methoxy- | |
| | ethanol | |
| 8 | MIBK | I |
| 9 | Toluene | I |
| 10 | Acetone | I |
| 11 | n-BuOH | II |
| 12 | MTBE | I |
| 13 | DMSO | |
| 14 | EtOH | I |
| 15 | EtOAc | I |
| 16 | Ethyl formate | III |
| 17 | Heptane | I |
| 18 | Isobutyl acetate | I |
| 19 | IPAC | I |
| 20 | n-Propanol | IV |
| 21 | IPA | I |
| 22 | Water | |
| 23 | MEK | V |
| 24 | THF | VI |
| 25 | 2-Methyl THF | V |
| 26 | 66% MTBE | I |
| | 20% MeOH | |
| | 10% EtOAc 4% | |
| | water | |
| 27 | 3% MeOH 0.3% | I |
| | water in EtOAc | |

TABLE 12

| Exp. No. | Solvent | Solid Form |
|---|---|---|
| 1 | MeCN | I |
| 2 | Chloroform | I |
| 3 | DCM | I |
| 4 | DMF | |
| 5 | 1,4-Dioxane | I |
| 6 | MeOH | |
| 7 | 2-Methoxy-ethanol | |
| 8 | MIBK | I |
| 9 | Toluene | I |
| 10 | Acetone | I |
| 11 | n-BuOH | |
| 12 | MTBE | I |
| 13 | DMSO | |
| 14 | EtOH | I |
| 15 | EtOAc | I |
| 16 | Ethyl formate | III |
| 17 | Heptane | I |
| 18 | Isobutyl acetate | I |
| 19 | IPAC | I |
| 20 | n-Propanol | |
| 21 | IPA | |
| 22 | Water | |
| 23 | MEK | V |
| 24 | THF | VI + |
| | | amorphous |
| 25 | 2-Methyl THF | V |
| 26 | 66% MTBE 20% | I |
| | MeOH | |
| | 10% EtOAc 4% water | |
| 27 | 3% MeOH 0.3% | I |
| | water in EtOAc | |

### Evaporation at 25° C. and 50° C.

Evaporation studies were carried out to identify the predominant crystalline form during uncontrolled precipitation. XRPD was used to study the solid-state morphology of the crystalline forms of the evaporation samples at 25±1° C. and 50±1° C. The results are presented in Table 13 (25±1° C.) and Table 14 (50±1° C.).

The Compound 1 HCl solids obtained after evaporation at 25° C. in IPA, methyl ethyl ketone (MEK), and a mixture of 66% MTBE 20% MeOH 10% EtOAc 4% water have a different XRPD patterns when comparing to that of the staring material Form I and those of Forms II-VI obtained after phase equilibration. This new crystalline form was named as Form VII.

The Compound 1 HCl solids obtained after evaporation at 50° C. in DCM, 1,4-dioxane, MIBK, MEK, ethyl formate, THF, and a mixture of 3% MeOH 0.3% water in EtOAc have the crystalline Form VII.

TABLE 13

| Exp. No. | Solvent | Solid Form |
|---|---|---|
| 1 | MeCN | I |
| 2 | Chloroform | I |
| 3 | DCM | I |
| 4 | DMF | |
| 5 | 1,4-Dioxane | |
| 6 | MeOH | I |
| 7 | 2-Methoxy-ethanol | |
| 8 | MIBK | |
| 9 | Toluene | |
| 10 | Acetone | I |
| 11 | n-BuOH | I |
| 12 | MTBE | |
| 13 | DMSO | |
| 14 | EtOH | I |
| 15 | EtOAc | |
| 16 | Ethyl formate | |
| 17 | Heptane | |
| 18 | Isobutyl acetate | |
| 19 | IPAC | |
| 20 | n-Propanol | I |
| 21 | IPA | VII |
| 22 | Water | |
| 23 | MEK | VII |
| 24 | THF | |
| 25 | 2-Methyl THF | |
| 26 | 66% MTBE | VII |
| | 20% MeOH | |
| | 10% EtOAc | |
| | 4% water | |
| 27 | 3% MeOH | |
| | 0.3% water | |
| | in EtOAc | |

TABLE 14

| Exp. No. | Solvent | Solid Form |
|---|---|---|
| 1 | MeCN | I |
| 2 | Chloroform | I |
| 3 | DCM | VII |
| 4 | DMF | |
| 5 | 1,4-Dioxane | VII |
| 6 | MeOH | I |
| 7 | 2-Methoxy- | |
| | ethanol | |
| 8 | MIBK | VII |
| 9 | Toluene | |
| 10 | Acetone | I |
| 11 | n-BuOH | II |
| 12 | MTBE | |
| 13 | DMSO | |
| 14 | EtOH | I |
| 15 | EtOAc | |
| 16 | Ethyl formate | VII |
| 17 | Heptane | |
| 18 | Isobutyl | |
| | acetate | |
| 19 | IPAC | |
| 20 | n-Propanol | |
| 21 | IPA | |
| 22 | Water | |
| 23 | MEK | VII |
| 24 | THF | VII |
| 25 | 2-Methyl | I |
| | THF | |
| 26 | 66% MTBE | I |
| | 20% MeOH | |
| | 10% EtOAc | |
| | 4% water | |
| 27 | 3% MeOH | VII |
| | 0.3% water | |
| | in EtOAc | |

### Anti-Solvent Addition

Saturated solutions and nearly saturated solutions of Compound 1 HCl (Form I) were prepared in the solvents listed in Table 15 at room temperature respectively. An anti-solvent was added dropwise to induce precipitation. The results are presented in Table 15, and Form VIII was obtained after adding 2-methyl THF to the DMF solution.

TABLE 15

| Exp. No. | Solvent (mL) | Anti-solvent (mL) | Solid Form |
|---|---|---|---|
| Compound 1 | N/A | N/A | I |
| HCl | | | |
| 1 | DMF (1.0) | MTBE (4) | VIII + I |
| 2 | DMF (1.0) | EtOAc (4) | I + VIII |
| 3 | DMF (1.0) | 2-Methyl THF (4) | VIII |
| 4 | MeOH (1.0) | MTBE (6) | I |
| 5 | MeOH (1.0) | EtOAc (10) | I |
| 6 | MeOH (1.0) | 2-Methyl THF (10) | V |

### Reverse Addition

Saturated solutions and nearly saturated solutions of Compound 1 HCl (Form I) were prepared in the solvents listed in Table 16 at 25° C. and added dropwise to a larger volume of a miscible anti-solvent. The results are presented in Table 16. In reverse addition experiments (Table 16), Form VII was identified from DMF-MTBE, DMF-EtOAc and DMF-Me-THF. Form V was identified from MeOH-MeTHF.

TABLE 16

| Exp. No. | Solvent (mL) | Anti-solvent (mL) | Solid Form |
|---|---|---|---|
| 1 | DMF (1.0) | MTBE (8) | VIII |
| 2 | DMF (1.0) | EtOAc (8) | VIII |
| 3 | DMF (1.0) | 2-Methyl THF (8) | VIII |
| 4 | MeOH (1.0) | MTBE (8) | I |
| 5 | MeOH (1.0) | EtOAc (8) | I |
| 6 | MeOH (1.0) | 2-Methyl THF (8) | V |

### Quench Cool of Saturated Solution

Saturated or nearly saturated solutions of Compound 1 HCl prepared at 25° C. were quenched cooled to about −20˜−30° C. to induce precipitation of higher energy forms. Representative solvents were chosen based on solubility data measured at 25° C. and 50° C. Form IX solids were observed in DMF, and the results were listed in Table 17.

TABLE 17

| Exp. No. | Solvent | Solid Form |
|---|---|---|
| Compound 1 HCl | NA | I |
| 1 | DMF | IX |
| 2 | MeOH | |
| 3 | n-BuOH | II |

### Characterization of the New Crystalline Forms

A total of nine crystalline Compound 1 HCl polymorphs were identified by standard polymorph screening methods, including the original crystalline form of Compound 1 HCl (Form I) and eight new crystalline forms (Forms II-IX). Form I was the most stable form of Compound 1 HCl and is a dihydrate. FIGS. 22-29 are XRPD diffractograms of Forms II-IX.

Form II was obtained by phase equilibration (at 25° C. in n-BuOH), evaporation (at 50° C. in n-BuOH) and quench cool (in n-BuOH) experiments (Tables 11, 17 and 17); Form III was obtained by phase equilibration (at 25° C. and 50° C.) in ethyl formate (Tables 11 and 12); Form IV was obtained by phase equilibration (at 25° C. in n-propanol) experiment (Table 11); Form V was obtained by phase equilibration (at 25° C. and 50° C. in MEK and 2-methyl THF, Tables 11 and 12), anti-solvent addition (adding 2-methyl to the MeOH solution of Compound 1 HCl, Table 15) and corresponding reverse addition (Table 16) experiments; Form VI was obtained by phase equilibration (at 25° C. and 50° C.) in THF (Tables 11 and 12); Form VII was obtained by evaporation in MEK (at 25° C. in and 50° C.), in IPA and a mixture of 66% MTBE 20% MeOH 10% EtOAc 4% water (at 25° C.), in DCM, 1,4-dioxane, MIBK, ethyl formate, THF, and a mixture of 3% MeOH 0.3% water in EtOAc (at 50° C.) (Tables 13 and 14); Form VIII was obtained in anti-solvent addition (adding 2-methyl THF to the DMF solution of Compound 1 HCl) and reverse addition (adding the DMF solution of Compound 1 HCl to 2-methyl THF, to EtOAc, and to MTBE) (Tables 15 and 16); Form IX was obtained in quench cool experiment in DMF (Table 17).

### Example 7. Salt Screening

### Compound 1 Hydrochloride Dihydrate

The mono-HCl salt of Compound 1 was prepared according to Example 2 and is a dihydrate.

Mono-HCl salt Form I was confirmed as a crystalline solid according to XRPD analysis. The XRPD pattern is shown in FIG. 1 and the peak data are provided in Table 1.

The DSC thermogram is shown in FIG. 2. It de-hydrated first below 150° C. and then followed by exothermal events of melting/decomposition at an onset temperature of about 265° C. with a peak temperature of about 271° C.

The TGA thermogram is shown in FIG. 3. Weight loss of ˜4.6% was observed below 100° C. due to loss of water. Weight loss of ˜11.4% between 150-300° C. was due to decomposition.

### Compound 1 Dihydrochloride

Preparation of Compound 1 dihydrochloride salt.

To a stirred solution of tert-butyl (1R,4R,5S)-5-(8-(2-cyanoethyl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-1-yl)-2-azabicyclo[2.1.1]hexane-2-carboxylate (0.500 g, 0.678 mmol) in Methanol (1.000 ml) (2 volume) and EtOAc (2 mL, 4 volume) at room temperature was added 12 M Hydrochloric acid in water (0.283 ml, 3.39 mmol) (37% HCl in water, 5 eq). The reaction mixture was heated at 50° C. for 1 h. Additional EtOAc (8 mL, 16 volume) was then added to precipitate more di-hydrochloride salt. The resulting slurry was heated at 50° C. for 15 min, then cooled to room temperature and stirred at for 1 h. The solid was collected by vacuum filtration and washed with EtOAc. It was dried at room temperature under the house vacuum overnight to give the product di-hydrochloride salt as a light yellow solid (0.470 g, 98.09% pure by HPLC @ 254 nm, containing 4.63% of water by KF, 92% yield). LCMS: 628.1, 630.1 (M+H). The hydrochloride salt ratio is 1.95 by chloride titration.

Dihydrochloride salt was confirmed as a crystalline solid according to XRPD analysis. The XRPD pattern is shown in FIG. 4 and the peak data are provided in Table 2.

The DSC thermogram is shown in FIG. 5. It de-hydrated first below 150° C. and then followed by exothermal events of melting/decomposition at an onset temperature of about 256° C. with a peak temperature of about 267° C.

The TGA thermogram is shown in FIG. 6. Weight loss of ˜4.5% was observed below 150° C. due to loss of water. The compound decomposes above 150° C. with weight loss of ˜16.6% up to 300° C.

### Compound 1 Fumarate

### Preparation of Compound 1 Fumarate Salt.

76.56 mg of free base was dissolved in 1 mL acetone in a 4 mL clear glass vial with stirring. To the solution, 17.43 mg of fumaric acid (1.23 eq) was added and mixed well. The solution was evaporated without cap at room temperature to dryness overnight. To the resulted oil/solution, 1 mL of EtOAc was added and heated at 60° C. with stirring for 1 h to solid out. The suspension was slurring at room temperature for 20 min. The solid of fumarate salt was collected by filtration, wash with EtOAc and vacuum dry at 50° C. for 1 h. The salt ratio between the free base and fumaric acid was determined to be 1.0 by NMR analysis. The water content is 5.17 by KF.

Fumarate salt was confirmed as a crystalline solid according to XRPD analysis. The XRPD pattern is shown in FIG. 7 and the peak data are provided in Table 3.

The DSC thermogram is shown in FIG. 8. It de-hydrated first below 150° C. and then followed by melting/decomposition at an onset temperature of about 199° C. with a peak temperature of about 200° C.

The TGA thermogram is shown in FIG. 9. Weight loss of ˜4.4% was observed below 100° C. due to loss of water. Fumarate salt is potentially a di-hydrate. The compound decomposes above 150° C. with weight loss of ˜10.0% up to 300° C.

### Compound 1 L-Tartrate

### Preparation of Compound 1 L-Tartrate Salt.

76.81 mg of free base was dissolved in 1 mL methanol in a 4 mL clear glass vial with stirring. To the solution, 23.83 mg of L-tartaric acid (1.3 eq) was added and mixed well. The solution was evaporated without cap at room temperature to ˜0.5 mL. Then 1 mL EtOAc was added and heated at 50° C. with stirring for 1 h to solid out. The suspension was stirring at room temperature for 2 h. The solid of L-tartrate salt was collected by filtration, wash with EtOAc and vacuum dry at 50° C. for 1 h. The salt ratio between the free base and L-tartaric acid was determined to be 1.2 by NMR analysis.

L-tartrate salt was confirmed as a crystalline solid according to XRPD analysis. The XRPD pattern is shown in FIG. 10 and the peak data are provided in Table 4.

The DSC thermogram is shown in FIG. 11. It de-hydrated first below 150° C. and then followed by melting/decomposition at an onset temperature of about 181° C. with a peak temperature of about 208° C.

The TGA thermogram is shown in FIG. 12. Weight loss of ˜4.0% was observed below 100° C. mainly due to loss of water. The compound decomposes above 100° C. with weight loss of ˜17.5% up to 300° C.

### Compound 1 Adipate

### Preparation of Compound 1 Adipate Salt.

80.15 mg of free base was dissolved in 0.5 mL acetone in a 4 mL clear glass vial with stirring. To the solution, 22.89 mg of adipic acid (1.23 eq) was added and heated at 60° C. to dissolve. The solution was evaporated without cap at room temperature to oil overnight. To the resulted oil, 1 mL EtOAc was added and heated at 60° C. to dissolve. The solution was evaporated without cap at room temperature to oil out. Then it was heated at 60° C. to solid out with addition of 1 mL EtOAc. The suspension was stirring at room temperature for 30 min. Adipate salt was collected by filtration, wash with EtOAc and vacuum dry at 50° C. for 30 min. The salt ratio between the free base and adipic acid was determined to be 1.3 by NMR analysis.

Adipate salt was confirmed as a crystalline solid according to XRPD analysis. The XRPD pattern is shown in FIG. 13 and the peak data are provided in Table 5.

The DSC thermogram is shown in FIG. 14. The DSC thermogram revealed first dehydration at an onset temperature of about 26° C. with a peak temperature of 88.9° C. followed by multiple endothermal events at peak temperatures of about 154° C., 189° C., and 199° C. due to melting/decomposition of the compound.

The TGA thermogram is shown in FIG. 15. Weight loss of 0.87% was observed below 100° C. The second weight loss of ˜15% between 150-300° C. is due to decomposition of the compound.

### Compound 1 Phosphate

### Preparation of Compound 1 Phosphate Salt.

74.40 mg of free base was dissolved in 0.4 mL acetone in a 4 mL clear glass vial with stirring. To the solution, 9.0 μL of 85% phosphoric acid (1.1 eq) was added with stirring to form sticky solid. Then 2 mL EtOAc was added and heated at 60° C. for 1 h to obtain good slurry. The suspension was stirring at room temperature for 30 min. Phosphate salt was collected by filtration, wash with EtOAc and vacuum dry at 50° C. for 30 min. The salt ratio between the free base and phosphoric acid was determined to be 1.8 by NMR analysis.

Phosphate salt was confirmed as a crystalline solid according to XRPD analysis. The XRPD pattern is shown in FIG. 16 and the peak data are provided in Table 6.

The DSC thermogram is shown in FIG. 17. It potentially de-hydrated/de-solvated first below 150° C. and then followed by exothermal events of melting/decomposition at an onset temperature of about 218° C. with a peak temperature of about 227° C.

The TGA thermogram is shown in FIG. 18. Weight loss of 5.4% was observed below 150° C. The second weight loss of ˜11.7% between 150-300° C. is due to decomposition of the compound.

### Compound 1 Free Base

Crystalline Compound 1 free base was characterized by XRPD, DSC and TGA. The XRPD pattern is shown in FIG. 19 and the XRPD data are provided in Table 7, which confirm that the free base is crystalline solid.

The DSC thermogram is shown in FIG. 20. The DSC thermogram revealed that the free base potentially de-hydrated first below 100° C. and then followed by melting/decomposition at an onset temperature of about 176° C. with a peak temperature of about 195° C.

The TGA thermogram is shown in FIG. 21. Weight loss of ˜1.2% was observed at below 150° C. The compound starts to decompose above 200° C. with weight of ˜4.8% up to 300° C.

### Example 8. Compound 1 Hydrochloride Dihydrate Single Crystal Determination

A suitable single crystal of Compound 1 hydrochloride dihydrate was selected and analyzed by single-crystal X-ray diffractometry. Standard uncertainty in this report is written in crystallographic parenthesis notation, e.g., 0.123(4) is equivalent to 0.123±0.004. The crystal system is monoclinic and the space group is P2₁. The cell parameters and calculated volume are: a=13.8490 (4) Å, b=8.0204 (2) Å, c=15.5461 (4) Å, α=90°, β=100.617 (3)°, γ=90°, V=1697.21 (8) Å³. The formula weight is 701.04 g mol⁻¹ with Z=2, resulting in a calculated density of 1.372 g cm⁻³. Further details of the crystal data and crystallographic data collection parameters are summarized in Table A. The quality of the structure obtained is high as indicated by the fit residual, R, of 0.0426 (4.26%). R-factors in the range 2%-6% are quoted to be the most reliably determined structures.

An atomic displacement ellipsoid drawing of Compound 1 HCl dihydrate is shown in FIG. 30. The asymmetric unit shown in FIG. 30 contains one Compound 1 cation, one chloride anion, and two water molecules.

The absolute structure can be determined through an analysis of anomalous X-ray scattering by the crystal. Anomalous scattering is assessed through the intensity differences between Friedel pairs. For the reflection data measured up to θₘₐₓ the Friedel coverage was 52.9%. A refined parameter x, known as the Flack parameter (H. D. Flack, et al., Acta Cryst., 1999, A55, 908-15; H. D. Flack, et al., G., J. Appl. Cryst., 2000, 33, 1143-48; H. D. Flack, Acta Cryst., 1983, A39, 876-881; S. Parsons, et al., Acta Cryst., 2013, B69, 249-259.)

encodes the relative abundance of the two components in an inversion twin. The structure contains a fraction 1−x of the model being refined, and x of its inverse. Provided that a low standard uncertainty is obtained, the Flack parameter should be close to 0 if the solved structure is correct, and close to 1 if the inverse model is correct. The measured Flack parameter for the structure of Compound 1 HCl dihydrate shown in FIG. 30 is 0.028(19), which indicates strong inversion-distinguishing power.

Additional information regarding the absolute structure can be assessed by applying Bayesian statistics to Bijvoet differences. This analysis provides a series of probabilities for different hypotheses of the absolute structure. This analysis results in the Hooft y parameter, which is interpreted in the same fashion as the Flack x parameter. In addition, this analysis results in three probabilities that the absolute structure is either correct, incorrect or a racemic twin. For the current data set the (Flack equivalent) Hooft y parameter is 0.001(14), the probability that the structure is correct is 1.000, and the probabilities that the structure is either incorrect or a racemic twin are both less than 10-200.

Therefore, the absolute configuration of the model in FIG. 30 is correct. This structure contains six chiral centers located at C22, C23, C26, C27, C29, and C31 (refer to FIG. 30) which bond in the S,R,R,R,R, and R configuration, respectively. The atropisomeric arrangement of the biphenyl rings is in the M (Rₐ) configuration. All chirality is consistent with the proposed configuration as shown throughout the present disclosure.

TABLE A
Crystal Data and Data Collection Parameters

| | |
|---|---|
| Empirical formula | C₃₅H₃₇Cl₃FN₅O₃ |
| Formula weight (g mol⁻¹) | 701.04 |
| Temperature (K) | 150 |
| Wavelength (Å) | 1.54184 |
| Crystal system | monoclinic |
| Space group | P2₁ |
| Unit cell parameters | |
| a = 13.8490(4) Å | α = 90° |
| b = 8.0204(2) Å | β = 100.617(3)° |
| c = 15.5461(4) Å | γ = 90° |
| Unit cell volume (Å³) | 1697.21(8) |
| Cell formula units, Z | 2 |
| Calculated density (g cm⁻³) | 1.372 |
| Absorption coefficient (mm⁻¹) | 2.848 |
| F(000) | 732 |
| Crystal size (mm³) | 0.28 × 0.05 × 0.03 |
| Reflections used for cell measurement | 4805 |
| θ range for cell measurement | 3.9200°-75.4780° |
| Total reflections collected | 8499 |
| Index ranges | −13 ≤ h ≤ 16; −9 ≤ k ≤ 8; −19 ≤/≤ 15 |
| θ range for data collection | θₘᵢₙ = 3.931°, θₘₐₓ = 75.800° |
| Completeness to θₘₐₓ | 96.4% |
| Completeness to θ_{full} = 67.684° | 99.9% |
| Absorption correction | multi-scan |
| Transmission coefficient range | 0.880-1.000 |
| Refinement method | full matrix least-squares on F² |
| Independent reflections | 5378 [Rᵢₙₜ = 0.0304, R_{σ} = 0.0488] |
| Reflections [ I > 2σ(I) ] | 4795 |
| Reflections/restraints/parameters | 5378/1/572 |
| Goodness-of-fit on F² | S = 1.05 |
| Final residuals [ I > 2σ(I) ] | R = 0.0426, R_{w} = 0.1170 |
| Final residuals [ all reflections ] | R = 0.0482, R_{w} = 0.1208 |
| Largest diff. peak and hole (e Å⁻³) | 0.315, −0.312 |
| Max/mean shift/standard uncertainty | 0.000/0.000 |
| Absolute structure determination | Flack parameter: 0.028(19) |
| | Hooft parameter: 0.001(14) |
| | Friedel coverage: 52.9% |

### Example 9. Compound 1 Hydrochloride Dihydrate Tablet Formulation

For simplicity, Compound 1 hydrochloride dihydrate is referred to as “Cmpd 1 HCl” in this Example.

A tablet formulation was prepared using the components and amounts listed in Table 18.

TABLE 18
Formulation for 25 mg, 100 mg and 200 mg Tablets (common blend):

| | |
|---|---|
| Material | Formulation (%, w/w) |

Intragranular material

| | |
|---|---|
| Cmpd 1 HCl | 37.79 |
| HPMC (Methocel E5 Premium LV Hydroxypropyl | 4.46 |
| Methylcellulose) | |
| Microcrystalline Cellulose Avicel PH101 | 23.52 |
| Mannitol (Pearlitol 50C) | 23.52 |
| Purified Water | N/A |
| Total Intragranular Materials | 89.29 |

Extragranular material

| | |
|---|---|
| Sodium Starch Glycolate JRS | 4.00 |
| Microcrystalline Cellulose Avicel PH102 | 4.21 |
| Colloidal Silicon Dioxide | 0.50 |
| Sodium Stearyl Fumarate JRS | 1.00 |
| Magnesium Stearate | 1.00 |
| Total Extragranular Materials | 10.71 |
| Total Materials | 100 |

Tablet weights and dimensions are listed in Table 19 and Table 19A.

TABLE 19
Tablet weight and dimensions:

| *Dose of | Tablet | Tooling size and | Hardness | Thickness |
|---|---|---|---|---|
| Compound 1 | weight(mg) | shape | (kp) | (mm) |
| 25 mg | 70 | 13/64 inch round | 3-7 | 3.3 |
| | | concave | | |
| 100 mg | 280 | 11/32 inch round | 8-12 | 4.8 |
| | | concave | | |
| 200 mg | 560 | 0.625 × 0.3125 oval | 13-17 | 5.9 |

*Doses of Compound 1 are provided as the free base equivalent.

The manufacturing steps for synthesis of the tablet of Table 19 is as follows:
Pass Cmpd 1 HCl through Comill equipped with 039R screen at 750 RPM
Pass HPMC, Mannitol and MCC PH101 sequentially through Comill
Blend all intragranular materials in a 20 L bin blender for 10 minutes at 15 RPM
Charge the pre-granulation blend into an appropriate wet granulator, perform wet granulation operation (impeller speed: 150 RPM/chopper speed: 1800 RPM/water pumping rate 75 g/minute with atomization air)
Pass wet granules through Comill equipped with 125R screen at 750 RPM
Dry wet granules in a fluid bed drier (target LOD<2.5%)
Pass dried granules through Comill equipped with 032R screen at 2200 RPM
Charge half of dried granules into a 10 L bin blender
Combine MCC PH102, Silicon Dioxide and Sodium Starch Glycolate together, pass through Comill equipped with 032R screen at 750 RPM, then add to the 20 L bin blender
Charge the other half of dried granules into a 20 L bin blender. Blend for 20 minutes at 15 RPM
Pass Sodium Stearyl Fumarate through a #40-mesh screen
Remove some blend from 10 L bin blender, which is 2 times of the quantity of Sodium Stearyl Fumarate. Mix it with Sodium Stearyl Fumarate in a plastic bag for 30 seconds.
Charge the Sodium Stearyl Fumarate mixture into the 20 L bin blender. Blend for 5 minutes at 15 RPM
Pass Magnesium Stearate through a #40-mesh screen
Remove some blend from 20 L bin blender, which is 2 times of the quantity of Magnesium Stearate. Mix it with Magnesium Stearate in a plastic bag for 30 seconds.
Charge the Magnesium Stearate mixture into the 20 L bin blender. Blend for 5 minutes at 15 RPM
Compress tablets from the appropriate amount of the blend to provide a tablet containing the dose of Compound 1 using a tablet press.

TABLE 19A
Tablet weight and dimensions:

| *Dose of | Tablet | Tooling size and | Hardness | Thickness |
|---|---|---|---|---|
| Compound 1 | weight(mg) | shape | (kp) | (mm) |
| 25 mg | 70 | 13/64 inch round | 3-7 | 3.2 |
| | | concave | | |
| 100 mg | 280 | 11/32 inch round | 8-12 | 4.5 |
| | | concave | | |
| 200 mg | 560 | 0.625 × 0.3125 oval | 13-18 | 5.5 |

*Doses of Compound 1 are provided as the free base equivalent.

The manufacturing steps for synthesis of the tablet of Table 19A is as follows:
Pass Cmpd 1 HCl through Flexsift equipped with 039R screen at 1000 RPM
Pass HPMC, Mannitol and MCC PH101 sequentially through Flexsift
Blend all intragranular materials in a 20 L bin blender for 10 minutes at 15 RPM
Charge the pre-granulation blend into an appropriate wet granulator, perform wet granulation operation (impeller speed: 150 RPM/chopper speed: 1800 RPM/water pumping rate 75 g/minute with atomization air)
Pass wet granules through Comill equipped with 125R screen at 750 RPM
Dry wet granules in a fluid bed drier (target LOD<2.5%)
Pass dried granules through Comill equipped with 032R screen at 2200 RPM
Charge half of dried granules into a 10 L bin blender
Combine MCC PH102, Silicon Dioxide and Sodium Starch Glycolate together, pass through Flexsift equipped with 039R screen at 1000 RPM, then add to the 20 L bin blender
Charge the other half of dried granules into a 20 L bin blender. Blend for 20 minutes at 15 RPM
Pass Sodium Stearyl Fumarate through Flexsift equipped with 039R screen at 1000 RPM,
Charge the Sodium Stearyl Fumarate into the 20 L bin blender. Blend for 5 minutes at 15 RPM
Pass Magnesium Stearate through Flexsift equipped with 039R screen at 1000 RPM
Charge the Magnesium Stearate into the 20 L bin blender. Blend for 5 minutes at 15 RPM
Compress tablets from the appropriate amount of the blend to provide a tablet containing the dose of Compound 1 using a tablet press.
   Supportive stability data are provided in Tables 20-23.

TABLE 20
Cmpd 1 HCl, 25 mg IR tablets at 25° C./60% RH

| | Acceptance | Time (Months) | | | | | |
|---|---|---|---|---|---|---|---|
| Test | Criterion | Initial^{a} | | 1 M | 2 M | 3 M | 6 M |
| Assay | 90.0% to | 97.0% | | 99.6 | 99.2 | | |
| | 110.0% | | | | | | |
| | of Label | | | | | | |
| | Claim | | | | | | |
| Content | USP<905> | Average = | 100.6 | Not tested | | | |
| Uniformity^{b} | | Std dev = | 0.4 | | | | |
| | | % RSD = | 0.4 | | | | |
| | | AV = | 0.9 | | | | |

Related Substances (RRT: % Area)

| Individual | ≤1.0% | Related | | Related | | Related | | | | |
|---|---|---|---|---|---|---|---|---|---|---|
| | | Compound A | | Compound A | | Compound A | | | | |
| | | RRT: 0.95: | | RRT 0.89: | | RRT 0.90: | | | | |
| | | 0.05% | | 0.12% | | 0.12% | | | | |
| | | Related | | Related | | Related | | | | |
| | | Compound B | | Compound B | | Compound B | | | | |
| | | RRT 1.11: | | RRT 1.11: | | RRT 1.12: | | | | |
| | | 0.07% | | 0.05% | | 0.07% | | | | |
| Total Degradants | ≤5.0 % | 0.12% | | 0.17% | | 0.19% | | | | |
| Water Content | Report | 4.0% | | 3.9% | | 4.4% | | 4.3% | | |
| | results | | | | | | | | | |
| | (% w/w) | | | | | | | | | |
| Dissolution | Report | Mean = | 98.0 | Mean = | 91.1 | NT^{c} | | NT^{c} | | NT^{c} |
| 5 min. (Cmpd 1) | Results | % RSD = | 2.1 | % RSD = | 4.8 | | | | | |
| Dissolution | Report | Mean = | 102.6 | Mean = | 100.1 | Mean = | 99.9 | Mean = | 100.5 | |
| 10 min. (Cmpd 1) | Results | % RSD = | 2.1 | % RSD = | 3.6 | % RSD = | 1.6 | % RSD = | 2.5 | |
| Dissolution | Report | Mean = | 104.2 | Mean = | 100.5 | Mean = | 100.9 | Mean = | 101.9 | |
| 20 min. (Cmpd 1) | Results | % RSD = | 1.7 | % RSD = | 4.2 | % RSD = | 2.7 | % RSD = | 1.4 | |
| Dissolution | Report | Mean = | 104.6 | Mean = | 102.2 | Mean = | 100.7 | Mean = | 101.4 | |
| 30 min. (Cmpd 1) | Results | % RSD = | 1.6 | % RSD = | 7.1% | % RSD = | 2.4 | % RSD = | 1.9 | |
| Dissolution | Report | Mean = | 104.9 | Mean = | 102.2 | Mean = | 102.8 | Mean = | 103.4 | |
| 45 min. (Cmpd 1) | Results | % RSD = | 1.7 | % RSD = | 5.3 | % RSD = | 1.8 | % RSD = | 2.1 | |
| Dissolution | Report | Mean = | 105.0 | Mean = | 100.2 | Mean = | 102.8 | Mean = | 103.4 | |
| Infinity (60 min.) | Results | % RSD = | 1.8 | % RSD = | 3.4 | % RSD = | 1.8 | % RSD = | 2.1 | |
| (Cmpd 1) | | | | | | | | | | |

NT: Not tested

TABLE 21
Cmpd 1, 25 mg IR tablets lot 40° C./75% RH

| | Acceptance | Time (Months) | | | | |
|---|---|---|---|---|---|---|
| Test | Criterion | Initialª | 1 M | 2 M | 3 M | 6 M |
| Assay | 90.0% to | 97.0 | 100.5 | 98.2 | | |
| | 110.0% | | | | | |
| | of Label | | | | | |
| | Claim | | | | | |

Related Substances (RRT: % Area)

| Individual | ≤1.0% | Related | | Related | | Related | | | | |
|---|---|---|---|---|---|---|---|---|---|---|
| | | Compound A | | Compound A | | Compound A | | | | |
| | | RRT: 0.95: | | RRT 0.89: | | RRT 0.90: | | | | |
| | | 0.05% | | 0.12% | | 0.12% | | | | |
| | | Related | | Related | | Related | | | | |
| | | Compound B | | Compound B | | Compound B | | | | |
| | | RRT 1.11: | | RRT 1.11: | | RRT 1.12: | | | | |
| | | 0.07% | | 0.05% | | 0.07% | | | | |
| Total Degradants | ≤5.0 % | 0.12% | | 0.17% | | 0.19% | | | | |
| Water Content | Report | 4.0% | | 4.0% | | 4.3% | | 4.3% | | |
| | results | | | | | | | | | |
| | (% w/w) | | | | | | | | | |
| Dissolution | Report | Mean | = 98.0 | Mean | = 92.1 | NT^{b} | | NT^{b} | | NT^{b} |
| 5 min. (Cmpd 1) | Results | % RSD | = 2.1 | % RSD | = 6.8 | | | | | |
| | mean and | | | | | | | | | |
| | % RSD | | | | | | | | | |
| Dissolution | Report | Mean | = 102.6 | Mean | = 100.4 | Mean = | 102.9 | Mean = | 100.5 | |
| 10 min. (Cmpd 1) | Results | % RSD | = 2.1 | % RSD | = 5.9 | % RSD = | 0.5 | % RSD = | 2.4 | |
| | mean and | | | | | | | | | |
| | % RSD | | | | | | | | | |
| Dissolution | Report | Mean | = 104.2 | Mean | 100.6 | Mean = | 102.6 | Mean = | 101.5 | |
| 20 min. (Cmpd 1) | Results | % RSD | = 1.7 | % RSD | = 4.5 | % RSD = | 0.9 | % RSD = | 0.8 | |
| | mean and | | | | | | | | | |
| | % RSD | | | | | | | | | |
| Dissolution | Report | Mean | = 104.6 | Mean | = 102.1 | Mean = | 103.0 | Mean = | 100.6 | |
| 30 min. (Cmpd 1) | Results | % RSD | = 1.6 | % RSD | = 3.8 | % RSD = | 1.0 | % RSD = | 0.6 | |
| | mean and | | | | | | | | | |
| | % RSD | | | | | | | | | |
| Dissolution | Report | Mean | = 104.9 | Mean | = 102.0 | Mean = | 103.8 | Mean = | 103.4 | |
| 45 min. (Cmpd 1) | Results | % RSD | = 1.7 | % RSD | = 3.8 | % RSD = | 2.1 | % RSD = | 2.2 | |
| | mean and | | | | | | | | | |
| | % RSD | | | | | | | | | |
| Dissolution | Report | Mean | = 105.0 | Mean | = 101.8 | Mean = | 103.9 | Mean = | 100.5 | |
| Infinity (Cmpd 1) | Results | % RSD | = 1.8 | % RSD | = 4.7 | % RSD = | 1.9 | % RSD = | 1.7 | |

mean and
% RSD

NT: Not tested

TABLE 22
Cmpd 1 100 mg IR tablets at 25° C./60% RH

| Criterion | Acceptance | Time (Months) | | | | | |
|---|---|---|---|---|---|---|---|
| Test | Criterion | Initial^{a} | | 1 M | 2 M | 3 M | 6 M |
| Assay | 90.0% to | 101.2 | | 99.3 | NT | | |
| | 110.0% | | | | | | |
| | of Label | | | | | | |
| | Claim | | | | | | |
| Content | USP<905>% | Average | = 100.3 | NT | | | |
| Uniformity^{b} | | Std Dev | = 0.1 | | | | |
| | | % RSD | = 0.1 | | | | |
| | | AV | = 0.2 | | | | |

Related Substances (RRT: % Area)

| Individual | ≤1.0% | Related | | RRT 0.89 | | NT | | |
|---|---|---|---|---|---|---|---|---|
| | | Compound A | | 0.11% | | | | |
| | | RRT 1.11: | | | | | | |
| | | 0.07% | | | | | | |
| | | Related | | | | | | |
| | | Compound B | | | | | | |
| | | RRT 1.26: | | | | | | |
| | | 0.05% | | | | | | |
| Total | ≤5.0 % | 0.12% | | 0.11% | | NT | 0.17% | |
| Degradants | | | | | | | | |
| Water Content | Report | 3.9% | | 3.9% | | NT | 4.0% | |
| | results | | | | | | | |
| | (% w/w) | | | | | | | |
| Dissolution | Report | Mean | = 92.2 | Mean | = 83.5 | NT | NT | |
| 5 min. | Results mean | % RSD | = 3.2 | % RSD | = 6.7 | | | |
| (Cmpd 1) | and % RSD | | | | | | | |
| Dissolution | Report | Mean | = 99.6 | Mean | = 95.6 | NT | Mean | = 97.7 |
| 10 min. | Results mean | % RSD | = 2.4 | % RSD | = 1.6 | | % RSD | = 2.3 |
| (Cmpd 1) | and % RSD | | | | | | | |
| Dissolution | Report | Mean | = 101.7 | Mean | = 100.7 | NT | Mean | = 102.4 |
| 20 min. | Results mean | % RSD | = 2.2 | % RSD | = 1.1 | | % RSD | = 1.1 |
| (Cmpd 1) | and % RSD | | | | | | | |
| Dissolution | Report | Mean | = 102.6 | Mean | = 103.4 | NT | Mean | = 102.9 |
| 30 min. | Results mean | % RSD | = 2.2 | % RSD | = 1.1 | | % RSD | = 1.6 |
| (Cmpd 1) | and % RSD | | | | | | | |
| Dissolution | Report | Mean | = 103.1 | Mean | = 102.5 | NT | Mean | = 103.1 |
| 45 min. | Results mean | % RSD | = 2.1 | % RSD | = 1.2 | | % RSD | = 1.0 |
| (Cmpd 1) | and % RSD | | | | | | | |
| Dissolution | Report | Mean | = 103.7 | Mean | = 101.6 | NT | Mean | = 104.1 |
| Infinity | Results mean | % RSD | = 1.9 | % RSD | = 0.9 | | % RSD | = 1.1 |

| | |
|---|---|
| (Cmpd 1) | and % RSD |

NT: Not tested

TABLE 23
Cmpd 1 100 mg IR tablets at 40° C./75% RH

| | Acceptance | Time (Months) | | | | |
|---|---|---|---|---|---|---|
| Test | Criterion | Initial^{a} | 1 M | 2 M | 3 M | 6 M |
| Assay | 90.0% to | 101.2 | 100.1 | NT | | |
| | 110.0% | | | | | |
| | of Label | | | | | |
| | Claim | | | | | |

Related Substances (RRT: % Area)

| Individual | ≤1.0% | Compound A | | RRT 0.89: | | NT | | |
|---|---|---|---|---|---|---|---|---|
| | | RRT 1.11: | | 0.12% | | | | |
| | | 0.07% | | Compound A | | | | |
| | | Compound B | | RRT 1.11: | | | | |
| | | RRT 1.26: | | 0.06% | | | | |
| | | 0.05% | | | | | | |
| Total | ≤5.0 % | 0.12% | | 0.18% | | NT | 0.19% | |
| Degradants | | | | | | | | |
| Water Content | Report results | 3.9% | | 3.7% | | NT | 3.9% | |
| | (% w/w) | | | | | | | |
| Dissolution | Report Results | Mean | = 92.2 | Mean | = 88.0 | NT | NT | |
| 5 min. | mean and | % RSD | = 3.2 | % RSD | = 0.5 | | | |
| (Cmpd 1) | % RSD | | | | | | | |
| Dissolution | Report Results | Mean | = 99.6 | Mean | = 94.7 | NT | Mean | = 98.8 |
| 10 min. | mean and | % RSD | = 2.4 | % RSD | = 1.1 | | % RSD | = 1.1 |
| (Cmpd 1) | % RSD | | | | | | | |
| Dissolution | Report Results | Mean | = 101.7 | Mean | = 100.8 | NT | Mean | = 102.4 |
| 20 min. | mean and | % RSD | = 2.2 | % RSD | = 1.5 | | % RSD | = 1.0 |
| (Cmpd 1) | % RSD | | | | | | | |
| Dissolution | Report Results | Mean | = 102.6 | Mean | = 101.7 | NT | Mean | = 102.5 |
| 30 min. | mean and | % RSD | = 2.2 | % RSD | = 1.0 | | % RSD | = 1.0 |
| (Cmpd 1) | % RSD | | | | | | | |
| Dissolution | Report Results | Mean | = 103.1 | Mean | = 101.1 | NT | Mean | = 103.2 |
| 45 min. | mean and | % RSD | = 2.1 | % RSD | = 1.0 | | % RSD | = 1.0 |
| (Cmpd 1) | % RSD | | | | | | | |
| Dissolution | Report Results | Mean | = 103.7 | Mean | = 100.6 | NT | Mean | = 103.3 |
| Infinity | mean and | % RSD | = 1.9 | % RSD | = 0.9 | | % RSD | = 1.0 |

| | |
|---|---|
| (Cmpd 1) | % RSD |

NT: Not tested

Ingredients for an alternative tablet formulation are listed in Table 24. Procedures and dosages for preparing tablets are analogous to those described above.

TABLE 24

| | |
|---|---|
| Material | Formulation (%, w/w) |

Intragranular material

| | |
|---|---|
| Cmpd 1 | 37.79 |
| HPMC(Methocel E5 Premium LV | 4.46 |
| Hydroxypropyl Methylcellulose) | |
| Microcrystalline Cellulose Avicel PH101 | 20.84 |
| Mannitol(Pearlitol 50C) | 20.84 |
| Koliphor P407 | 5.36 |
| Purified Water | N/A |
| Total Intragranular Materials | 89.29 |

Extragranular material

| | |
|---|---|
| Sodium Starch Glycolate JRS | 4.00 |
| Microcrystalline Cellulose Avicel PH102 | 4.21 |
| Colloidal Silicon Dioxide | 0.50 |
| Sodium Stearyl Fumarate JRS | 1.00 |
| Magnesium Stearate | 1.00 |
| Total Extragranular Materials | 10.71 |
| Total Materials | 100 |

Various modifications of the disclosure, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A compound that is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile, or a pharmaceutically acceptable salt thereof, wherein the compound is crystalline.

2. The compound or pharmaceutically acceptable salt thereof of claim 1, which is a pharmaceutically acceptable salt.

3. (canceled)

4. The compound or pharmaceutically acceptable salt thereof of claim 1, which is a solvate.

5. The compound or pharmaceutically acceptable salt thereof of claim 1, which is a hydrate.

6. The compound or pharmaceutically acceptable salt thereof of claim 1, which is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride.

7-8. (canceled)

9. The compound or pharmaceutically acceptable salt thereof of claim 6, which is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate.

10. The compound of claim 6, which is Form I.

11. The compound or pharmaceutically acceptable salt thereof of claim 6, which is characterized by an XRPD diffractogram having at least one of the following peaks expressed in degrees-2-theta at angles (±0.2 degrees) of 5.7, 7.8, 13.5, 17.2, 19.2, 20.5, 22.8, 23.1, 23.5, and 26.0.

12. The compound or pharmaceutically acceptable salt thereof of claim 10, which is characterized by an XRPD diffractogram having at least three of the following peaks expressed in degrees-2-theta at angles (±0.2 degrees) of 5.7, 7.8, 13.5, 17.2, 19.2, 20.5, 22.8, 23.1, 23.5, and 26.0.

13. The compound or pharmaceutically acceptable salt thereof of claim 6, which is characterized by an XRPD diffractogram having peaks expressed in degrees-2-theta at angles (±0.2 degrees) of 5.7, 7.8, and 13.5.

13-20. (canceled)

21. The compound or pharmaceutically acceptable salt thereof of claim 6, comprising crystals that are monoclinic.

22-24. (canceled)

25. The compound or pharmaceutically acceptable salt thereof of claim 6, which is Form II.

26. The compound or pharmaceutically acceptable salt thereof of claim 25, which is characterized by an XRPD diffractogram having at least one of the following peaks expressed in degrees-2-theta at angles (±0.2 degrees) of 5.8, 7.6, 11.4, 12.5, 14.4, 17.9, and 25.3.

27. (canceled)

28. The compound or pharmaceutically acceptable salt thereof of claim 25, which is characterized by an XRPD diffractogram having peaks expressed in degrees-2-theta at angles (±0.2 degrees) of 7.6, 12.5, and 17.9.

29-45. (canceled)

46. The compound or pharmaceutically acceptable salt thereof of claim 1, which is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile dihydrochloride.

47. (canceled)

48. The compound or pharmaceutically acceptable salt thereof of claim 46, which is characterized by an XRPD diffractogram having at least three of the following peaks expressed in degrees-2-theta at angles (±0.2 degrees) of 5.6, 5.9, 10.7, 12.0, 12.8, 13.2, 15.6, 15.9, 18.5, 20.1, 22.1, and 23.9.

49. The compound or pharmaceutically acceptable salt thereof of claim 48, which is characterized by an XRPD diffractogram having peaks expressed in degrees-2-theta at angles (±0.2 degrees) of 5.6, 5.9, and 22.1.

50-57. (canceled)

58. The compound or pharmaceutically acceptable salt thereof of claim 1, which is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile fumarate.

59. The compound or pharmaceutically acceptable salt thereof of claim 58, which is characterized by an XRPD diffractogram having at least one of the following peaks expressed in degrees-2-theta at angles (±0.2 degrees) of 12.9, 15.1, 17.3, 17.7, 19.8, 20.0, 20.8, 22.0, and 23.7.

60. The compound or pharmaceutically acceptable salt thereof of claim 58, which is characterized by an XRPD diffractogram having at least three of the following peaks expressed in degrees-2-theta at angles (±0.2 degrees) of 12.9, 15.1, 17.3, 17.7, 19.8, 20.0, 20.8, 22.0, and 23.7.

61. The compound or pharmaceutically acceptable salt thereof of claim 60, which is characterized by an XRPD diffractogram having peaks expressed in degrees-2-theta at angles (±0.2 degrees) of 12.9, 17.7, and 23.7.

62-70. (canceled)

71. The compound or pharmaceutically acceptable salt thereof of claim 1, which is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile L-tartrate.

72. The compound or pharmaceutically acceptable salt thereof of claim 71, which is characterized by an XRPD diffractogram having at least one of the following peaks expressed in degrees-2-theta at angles (±0.2 degrees) of 9.3, 16.7, 18.6, 19.1, 19.4, 19.6, 22.3, 22.6, and 24.6.

73. The compound or pharmaceutically acceptable salt thereof of claim 71, which is characterized by an XRPD diffractogram having at least three of the following peaks expressed in degrees-2-theta at angles (±0.2 degrees) of 9.3, 16.7, 18.6, 19.1, 19.4, 19.6, 22.3, 22.6, and 24.6.

74. The compound or pharmaceutically acceptable salt thereof of claim 73, which is characterized by an XRPD diffractogram having peaks expressed in degrees-2-theta at angles (±0.2 degrees) of 9.3, 18.6, and 24.6.

75-83. (canceled)

84. The compound or pharmaceutically acceptable salt thereof of claim 1, which is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile adipate.

85. The compound or pharmaceutically acceptable salt thereof of claim 84, which is characterized by an XRPD diffractogram having at least one of the following peaks expressed in degrees-2-theta at angles (±0.2 degrees) of 6.3, 9.1, 10.1, 18.2, 19.2, 24.3, 34.1, and 49.8.

86. The compound or pharmaceutically acceptable salt thereof of claim 84, which is characterized by an XRPD diffractogram having at least three of the following peaks expressed in degrees-2-theta at angles (±0.2 degrees) of 6.3, 9.1, 10.1, 18.2, 19.2, 24.3, 34.1, and 49.8.

87. The compound or pharmaceutically acceptable salt thereof of claim 86, which is characterized by an XRPD diffractogram having peaks expressed in degrees-2-theta at angles (±0.2 degrees) of 6.3, 9.1, and 24.3.

88-95. (canceled)

96. The compound or pharmaceutically acceptable salt thereof of claim 1, which is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile phosphate.

97. The compound or pharmaceutically acceptable salt thereof of claim 96, which is characterized by an XRPD diffractogram having at least one of the following peaks expressed in degrees-2-theta at angles (±0.2 degrees) of 4.5, 13.5, 13.7, 16.5, 16.9, 18.1, 21.5, 23.5, and 24.4.

98. The compound or pharmaceutically acceptable salt thereof of claim 96, which is characterized by an XRPD diffractogram having at least three of the following peaks expressed in degrees-2-theta at angles (±0.2 degrees) of 4.5, 13.5, 13.7, 16.5, 16.9, 18.1, 21.5, 23.5, and 24.4.

99. The compound or pharmaceutically acceptable salt thereof of claim 98, which is characterized by an XRPD diffractogram having peaks expressed in degrees-2-theta at angles (±0.2 degrees) of 4.5, 13.5, and 13.7.

100-108. (canceled)

109. The compound or pharmaceutically acceptable salt thereof of claim 1, which is 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

110. The compound or pharmaceutically acceptable salt thereof of claim 109, which is characterized by an XRPD diffractogram having at least one of the following peaks expressed in degrees-2-theta at angles (±0.2 degrees) of 5.7, 7.1, 8.3, 13.0, 13.5, 14.3, 15.5, 18.7, 20.8, and 22.7.

111. The compound or pharmaceutically acceptable salt thereof of claim 109, which is characterized by an XRPD diffractogram having at least three of the following peaks in degrees-2-theta at angles (±0.2 degrees) of 5.7, 7.1, 8.3, 13.0, 13.5, 14.3, 15.5, 18.7, 20.8, and 22.7.

112. The compound or pharmaceutically acceptable salt thereof of claim 111, which is characterized by an XRPD diffractogram having peaks expressed in degrees-2-theta at angles (±0.2 degrees) of 5.7, 7.1, and 8.3.

113-121. (canceled)

122. The compound or pharmaceutically acceptable salt thereof of claim 1, wherein the 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

123. (canceled)

124. A pharmaceutical composition comprising:
a) the compound or pharmaceutically acceptable salt thereof of claim 1;
b) a disintegrant;
c) a binder;
d) an anti-caking agent; and
e) a lubricant.

125. The pharmaceutical composition of claim 124, wherein the composition further comprises
f) an additive or a second lubricant.

126-130. (canceled)

131. A pharmaceutical composition comprising:
a) the compound or pharmaceutically acceptable salt thereof of claim 1;
b) sodium starch glycolate;
c) MCC PH102;
d) colloidal silicon dioxide;
e) sodium stearyl fumarate; and
f) magnesium stearate.

132. A dosage form comprising the compound or pharmaceutically acceptable salt thereof of claim 1.

133-137. (canceled)

138. A method of treating cancer in a subject in need thereof comprising administering to the subject a therapeutically effective amount of the compound or pharmaceutically acceptable salt thereof of claim 1.

139. (canceled)

140. The method of claim 138, wherein the cancer is associated with expression or activity of a KRAS protein having a G12D mutation.

141. A method for treating a cancer in a subject comprising identifying that the subject is in need of treatment of a cancer and that abnormally proliferating cells of the cancer comprise KRAS having a G12D mutation, and administering to the subject a therapeutically effective amount of the compound or pharmaceutically acceptable salt thereof of claim 1.

142. The method of claim 141, wherein the cancer is colorectal cancer, pancreatic cancer, or lung cancer.

143. (canceled)

144. The method of claim 141, wherein the cancer is pancreatic ductal cancer.

145. (canceled)

146. The method of claim 141, wherein the cancer is non-small cell lung cancer (NCSLC).

147. The method of claim 138, wherein the cancer is metastatic.

148. The method of claim 138, wherein abnormally proliferating cells of the cancer comprise KRAS having a G12D mutation.

149. A process for preparing 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile hydrochloride dihydrate comprising reacting 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile with aqueous hydrochloric acid.

150-157. (canceled)

158. The process of claim 149, wherein the 3-(1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile is 3-((Rₐ)-1-((1R,4R,5S)-2-azabicyclo[2.1.1]hexan-5-yl)-2-((1R,3R,5R)-2-(cyclopropanecarbonyl)-2-azabicyclo[3.1.0]hexan-3-yl)-7-(2,3-dichlorophenyl)-6-fluoro-4-methyl-1H-pyrrolo[3,2-c]quinolin-8-yl)propanenitrile.

159. (canceled)
